(19) United States
(12) Patent Application Publication
(10) Pub. No.: US 2015/0241410 A1
(43) Pub. Date: Aug. 27, 2015

(54) INHIBITORS OF CENTRAL NERVOUS SYSTEM VASOACTIVE INHIBITORY PEPTIDE RECEPTOR 2

(71) Applicant: The Trustees Of Columbia University In The City Of New York, New York, NY (US)

(72) Inventors: Maria Karayiorgou, Riverdale, NY (US); Joseph A. Gogos, Riverdale, NY (US); Jonathan Javitch, New Rochelle, NY (US); Talia Atkin, New York, NY (US)

(21) Appl. No.: 14/707,981

(22) Filed: May 8, 2015

Related U.S. Application Data
(63) Continuation of application No. PCT/US2013/069741, filed on Nov. 12, 2013.
(60) Provisional application No. 61/724,751, filed on Nov. 9, 2012.

(51) Int. Cl. G01N 33/50

(52) U.S. Cl. CPC G01N 33/502; G01N 2333/726; G01N 2800/302

(57) ABSTRACT

The present invention relates to compounds that inhibit VIPR₂ in the CNS, pharmaceutical compositions comprising said compounds, and methods of using such compounds and compositions in the treatment of a CNS disorder such as a behavioral disorder, including but not limited to schizophrenia.

### PRIORITY CLAIM

This application is a continuation of International Patent Application Serial No. PCT/U.S. Ser. No. 13/069,741, filed Nov. 12, 2013, which claims priority to U.S. Provisional Application Ser. No. 61/724,751, filed Nov. 9, 2012, priority to each of which is claimed, and the contents of each of which is incorporated by reference in its entirety herein.

### GRANT INFORMATION

This invention was made with government support under grant numbers 5RC1 MH088263-02 and 5R01MH067068-10 awarded by the National Institutes of Health. The government has certain rights in the invention.

### 1. INTRODUCTION

The present invention relates to small molecule inhibitors of central nervous system vasoactive inhibitory peptide receptor 2.

### 2. BACKGROUND OF THE INVENTION

Schizophrenia is a devastating disorder affecting 1% of the population with an annual economic burden of $62.7 billion (Wu et al., 2005, J Clin Psychiatry. 66:1122-1129). Current therapies lead to only a 15% sustained recovery rate over a 5 year period (Robinson et al., 2004, Am J Psychiatry. 161:473-479). Current drug treatments target the dopamine system, have many off-target effects and show only a 15% success rate (Vacic et al., 2011, Nature 471: 499-503; Robinson et al., 2004, Am J Psychiatry. 161:473-479).

Vasoactive intestinal peptide (VIP) is a basic 28 amino acid-peptide which is a member of a family of homologous peptides which includes glucagon. These peptides bind to a family of class II G protein-coupled receptors which themselves share homology. VIP, for example, is capable of binding to receptors VIPR₁, VIPR₂ and PAC. VIPR₂ is a 7-transmembrane™-G-protein-coupled receptor (GPCR) which stimulates adenylate cyclase via coupling to adenylate cyclase-stimulating G alpha protein, Gs, in addition to other transduction pathways, such as Ca²⁺ via coupling to G_{αi} and G_{αq} (Dickson et al., 2006, Neuropharmacology. 51:1086-1098) and phospholipase D (McCulloch et al., 2000, Ann N Y Acad Sci. 921:175-185). VIPR₂ activation is terminated via phosphorylation and the recruitment of β-arrestin for its internalisation and deactivation (Langer et al., 2007, Biochem Soc Trans. 35: 724-728). The VIPR₂ receptor is expressed in multiple brain regions associated with cognition and behavior, including the hippocampus, cerebral cortex, periventricular nucleus, suprachiasmatic nucleus, thalamus, hypothalamus, and amygdala (Sheward et al., 1995, Neuroscience. 67:409-418; Lutz et al., 1993, FEBS Lett. 334:3-8; Vertongen et al., 1998, Ann N Y Acad Sci. 865:412-415; Piggins, 2011, Nature 471:455-456).

Copy number variations involving the gene VIPR2, which encodes VIPR₂ (also known as “VPAC2”), have been linked to schizophrenia in a subset of patients (Vacic et al., 2011, Nature 471: 499-503; International Patent Application No. PCT/US2012/020683, published as WO2012/094681; International Patent Application No. PCT/US2012/023445, published as WO2012/106404). In particular, these copy number variations tend to result in increased expression of VIPR₂ and consequently increased VIPR₂ activity.

The biological functions of VIPR₂ are not completely understood. The removal of VIPR₂ function in VIPR₂-knockout mice resulted in decreased rhythmicity in brain suprachiasmatic neurons and a reduced behavioral circadian rhythm (Harnnar et al., 2002, Cell 109:497-508), altered immune hypersensitivity (Goetzl et al., 2001, Proc. Natl. Acad. Sci. U.S.A. 98:13854-13859) and an increased basal metabolic rate (Asnicar et al., 2002, Endocrinol. 143:3994-4006).

A few small molecules and peptides are known which act as inhibitors of VIPR₂ (Chu et al., 2010, Molecular Pharmacol. 77:95-101; Morena et al., 2000, Peptides 21:1543-1549). Chu et al. supra reported that screening 1.67 million compounds identified a single compound, (“Compound 1”) having the following structure, as an inhibitor of VIPR₂.

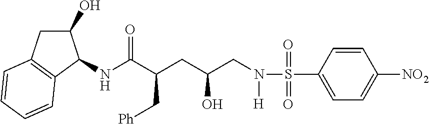 [SMILES: O=C(N[C@H]1c2ccccc2C[C@H]1O)[C@H](Cc1ccccc1)C[C@H](O)CNS(=O)(=O)c1ccc([N+](=O)[O-])cc1]

Compound 1 was reported to inhibit VIPR₂-mediated cAMP accumulation (IC₅₀ of 3.8 μM) and ligand-activated β-arrestin 2 binding (IC₅₀ of 2.3 μM; Chu et al., 2010, Molecular Pharmacol. 77:95-101). Compound 1 was observed to be highly specific for VIPR₂ with no detectable agonist or antagonist activities for VPAC1 or PAC1. Notably, a small structural change in Compound 1 (to form Compound 2 of Chu et al., supra) resulted in a substantial decrease in activity. Features of these known inhibitors indicate that compounds having improved blood-brain barrier penetration would be desirable to improve therapeutic efficacy as selective central nervous system (“CNS”) VIPR₂ inhibitors.

### 3. SUMMARY OF THE INVENTION

The present invention relates to compounds that inhibit VIPR₂ in the CNS, pharmaceutical compositions comprising said compounds, and methods of using such compounds and compositions in the treatment of a subject having or at risk of developing a CNS disorder. The compounds of the invention are similar to, but different from, Compounds 1 and 2 of Chu et al., 2010, Molecular Pharmacol. 77:95-101, and, in certain non-limiting embodiments, exhibit advantages such as enhanced stability, greater inhibitory activity and/or properties which would improve penetration of the blood-brain barrier and therefore provide greater availability to the CNS.

In certain embodiments, the present application provides for methods of inhibiting VIPR₂ activity in a cell expressing VIPR₂ by contacting a compound of the present application to the cell in an amount effective to inhibit or reduce VIPR2 activity.

In certain embodiments, the present application provides for methods of inhibiting VIPR₂ activity in a subject by administering a compound of the present application to the subject in an amount effective to inhibit or reduce VIPR2 activity.

In certain embodiments, the compound is administered to a subject or contacted to a cell in an amount effective to reduce or inhibit the ability of VIPR2 protein to activate cyclic-AMP signaling, for example, cyclic-AMP accumulation, or protein kinase A (PKA) activation.

In certain embodiments, the compound is administered to a subject or contacted to a cell in an amount effective to reduce or inhibit the ability of VIPR2 protein to bind to VIP.

In certain embodiments, the compound is administered to a subject or contacted to a cell in an amount effective to reduce or inhibit the ability of VIPR2 protein to regulate synaptic transmission, for example, increase or decrease synaptic transmission, between cells. In certain embodiments, the cells are in the hippocampus.

In certain embodiments, the compound is administered to a subject or contacted to the cell in an amount effective to reduce or inhibit the ability of VIPR2 protein to promote proliferation of neural progenitor cells, for example, in the dentate gyrus.

In certain embodiments, the compound is administered to a subject or contacted to a cell in an amount effective to reduce or inhibit the ability of VIPR2 protein to modulate circadian oscillations in, for example, the suprachiasmatic nucleus.

In certain embodiments, the compound is administered to a subject in an amount effective to treat a CNS disorder. In certain embodiments the CNS disorder is a psychiatric disorder, a neurodevelopmental disorder, or a behavioral disorder.

In certain embodiments, the compound is administered to a subject in an amount effective to treat a psychiatric disorder. In certain embodiments the psychiatric disorder is schizophrenia. In certain embodiments the psychiatric disorder is bipolar disorder, borderline personality disorder, schizoid disorder, major depression or obsessive compulsive disorder, or a disorder which combines features of the foregoing disorders.

In certain embodiments, the compound is administered to a subject in an amount effective to treat a neurodevelopmental disorder. In certain embodiments, the neurodevelopmental disorder is an autism spectrum disorder, for example autism, Aspergers syndrome. childhood disintegrative disorder, Rett syndrome, or pervasive developmental disorder not otherwise specified.

In certain embodiments, the compound is administered to a subject in an amount effective to treat or reduce the risk of occurrence of a behavioral disorder. In certain embodiments, the behavioral disorder is a sleep disorder such as insomnia, narcolepsy, or sleep deprivation.

The present invention also relates to methods for identifying an antagonist or agonist of VIPR2 through the use of a VIPR2 cellular assay utilizing cells that express a recombinant VIPR2 protein, but which do not express endogenous VIPR2.

In certain embodiments, a candidate compound can be identified as a VIPR2 antagonist through use of the VIPR2 cellular assay, wherein increasing concentrations of the candidate compound inhibits VIPR2 activity in the presence of a constant concentration of VIPR2 agonist.

In certain embodiments, the VIPR2 cellular assay measures cAMP activity as a measurement of VIPR2 activation. In certain embodiments, the VIPR2 cellular assay measures the level of β-arrestin recruited to the recombinant VIPR2 protein as a measurement of VIPR2 activation.

Compounds of the invention include compounds according to Formulas I-XXVII, below. Non-limiting examples of compounds of the invention are set forth in Tables 1, 2, 3 and 4 below.

### 4. BRIEF DESCRIPTION OF THE FIGURES

FIG. 1A-C. Schematic of BRET-based VIPR2 activation assays. Two methods have been developed to allow detection of VIPR2 activation. (A) Cell lines expressing both the VIPR₂ receptor and CAMYEL. In response to elevated cAMP levels, there is a conformational change in Epac and therefore a change in the proximity of the fused chromophores, Rluc8 and YFP. (B) BRET recruitment of mVenus—arrestin to VIPR2-Rluc8. (C) Results showing VIP responses alone and a rightward shift in the presence of increasing doses of Compound 1 (left). Compound F (Table 2, below) has a greater effect on VIPR₂ inhibition than Compound 1 (right).

FIG. 2. Schematic of BAC recombineering strategy for design of Vipr2 transgenic mice. The BAC clone is identified using the UCSC genome browser and obtained from BACPAC-CHORI. For the BAC recombination cassette, a LNL cassette is used with 50-bp fragments as homology arms a and b. Using homologous recombination in SW106 bacterial lines the start codon of Exon1 of ZFP-386 and the promoter region is deleted.

FIG. 3. Synthetic Scheme 1.

FIG. 4. BRET response, indicating raised levels of cAMP levels in CAMYEL CHO and HEK293 cells transiently transfected either alone or with the VIPR2 receptor. On treatment with increasing concentrations of VIP a response is elicited in HEK293 cells absent of VIPR2 transient transfection whereas no effect is seen in CHO cells in the absence of VIPR2 transfection. Both cell types expressing VPAC2 show dose response curves in response to VIP.

FIG. 5A-B. Results of BRET-based VIPR2 activation assays in cells expressing both the VIPR2 receptor and CAMYEL (see FIG. 1A for schematic). a) Inhibitory dose-response curves on treatment of CHO cells with increasing concentrations of a batch of antagonists against a background of VIP activation (VIP=5 nM). Increased inhibitory activity is represented by the leftward shift of the antagonist dose-response curve, where lower concentrations of antagonist are required to elicit an equal response in inhibition of VIP activation of cAMP levels. b) IC₅₀ values for various compounds showing the discovery of a compound (K) with a significantly improved activity as an antagonist at VIPR2.

FIG. 6A-B. (A) Schematic of BRET-based VIPR2 activation assay. BRET recruitment of mVenus-beta-arrestin to VIPR2-Rluc8, as described by Example 3. (B) Results of BRET recruitment of mVenus-β-arrestin to VIPR2-Rluc8. Inhibitory dose-response curves on treatment of CHO cells with increasing concentrations of a batch of antagonists against a background of VIP activation (VIP=5 nM).

FIG. 7. Assay demonstrating the expression of VIPR1 receptors together with CAMYEL therefore enabling the detection of compound specificity of VIPR2 antagonists to VIPR2 and absence of effect on the VIPR1 receptor through stimulation of cAMP.

### 5. DETAILED DESCRIPTION OF THE INVENTION

For purposes of clarity of disclosure and not by way of limitation, the detailed description of the invention is divided into the following subsections:
(i) compounds of the invention;
(ii) assays;
(iii) animal model systems; and
(iv) methods of treatment.

### 5.1 Compounds of the Invention

A compound of the invention has one of general formulas I-X as follows:

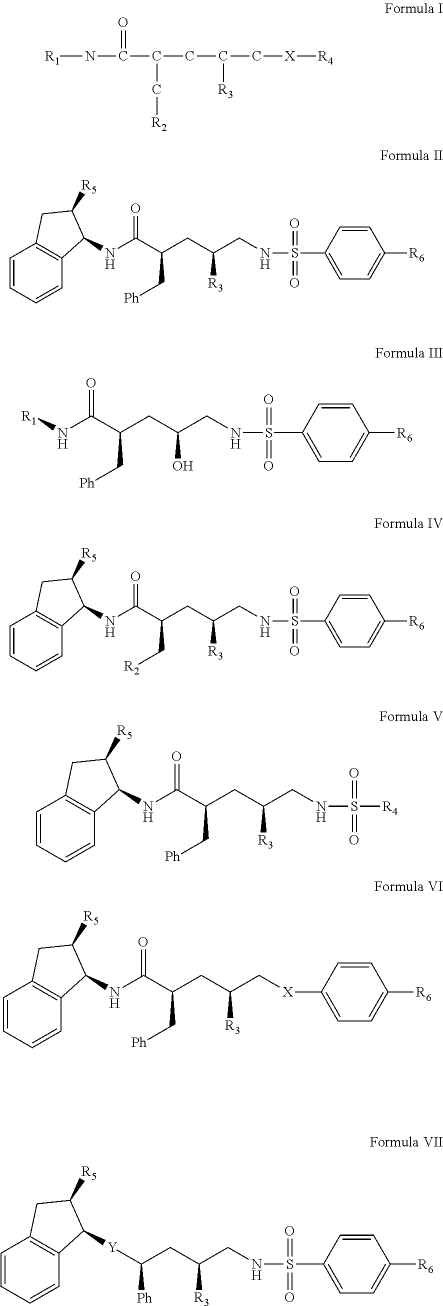

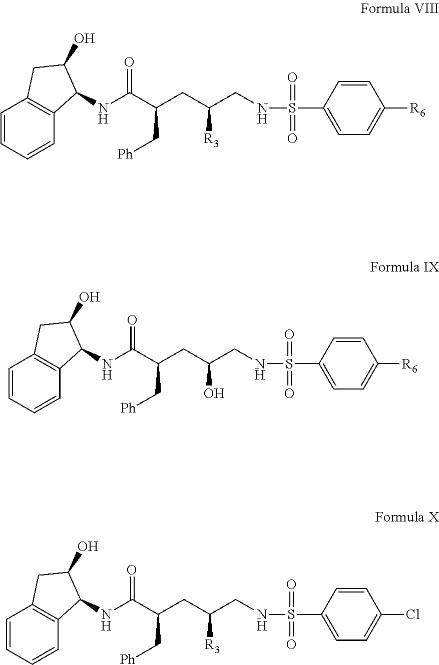

In the above formulas I-X:

R, can be substituted or unsubstituted aminoindanol, substituted or unsubstituted cyclic or acyclic alkyl (where cyclic alkyl can have 3-7 carbon atoms), substituted or unsubstituted aryl or heteroaryl, or mono or poly-substituted phenyl, where substitutents, if present, can be OH, F, Cl, C₁-C₄ alkyl, C₁-C₄ alkoxy, or C₁-C₄ alkyl ester or combinations thereof. Where R₁ is aminoindanol, the aminoindanol may be (1R,2S)(+)(cis) aminoindanol, or may be (1S,2R)(−)(cis) aminoindanol, or may be (1R,2R)(−)(trans) aminoindanol, or may be (1S,2S)(+)(trans) aminoindanol.

R₂ can be phenyl, pyridinyl or H.

R₃ and R₅ can be the same or different and can be H, OH, F, NH₂, CH₃, carbonyl, methylene, or difluoromethylene.

R₄ can be substituted or unsubstituted cyclic or acyclic alkyl (where cyclic alkyl can have 3-7 carbon atoms), substituted or unsubstituted aryl or heteroaryl, or mono or poly-substituted phenyl, where substitutents, if present, can be C₁-C₄ alkyl, C₁-C₄ alkoxy, or C₁-C₄ alkyl ester, methyl, propyl, isopropyl, ethyl, methoxy, ethyoxy, nitrile, F, Cl, CF₃ or combinations thereof.

In certain non-limiting embodiments, R₅ can be a substituted amine, which can optionally be a cyclic or aryl-fused amine.

R₆ can be C₁-C₄ alkyl, C₁-C₄ alkoxy, or C₁-C₄ alkyl ester, methyl, propyl, isopropyl, ethyl, methoxy, ethyoxy, nitrile, F, Cl, or CF₃ and in certain embodiments R₆ is not NO₂ or C(CH₃)₃.

X can be sulfonamide where the amide can be substituted or unsubstituted, reversed sulfonamide (as used herein, where a function group G is listed followed by a reference to “reversed” G, this means that the group is present in the compound in the reversed orientation; for example —C—O— reversed is —O—C—) where the amide can be substituted or unsubstituted, amide, reversed amide, ketone, alcohol or urea, where substitutents, if present, can be OH, F, Cl, C₁-C₄ alkyl, C₁-C₄ alkoxy, or C₁-C₄ alkyl ester or combinations thereof.

Y can be an amide, reversed amide, ketone, alcohol or urea, where the amide may optionally comprise an alkylated nitrogen, for example a C₁-C₄ alkylated nitrogen.

In certain embodiments, a compound of the invention has one of general formulas XI-XXI as follows:

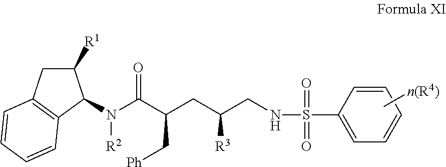

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-CS)cycloalkyl(C1-C5)alkyl, where R is alkyl, optionally substituted by one to three F;

n is 0, 1, 2 or 3.

Two R⁴ groups of Formula XI may be cyclized to form an infused ring.

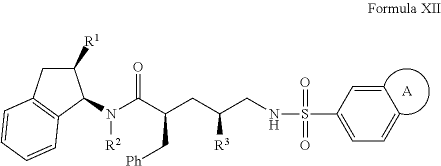

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Ring A is a saturated or unsaturated 5- or 6-membered cyclic, heterocyclic or heteroaryl group containing 0, 1, 2 or 3 of C, O, N or S.

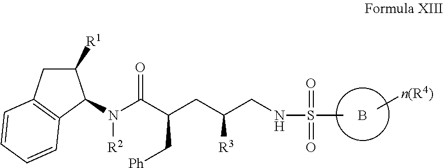

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Ring B is a 5- or 6-membered cyclic, heterocyclic, aryl or heteroaryl group containing 0, 1, 2 or 3 of C, O, N or S.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F;

and n is 0, 1, 2 or 3.

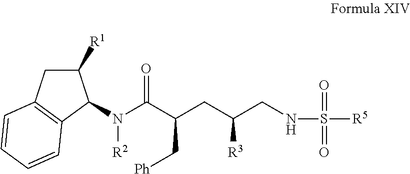

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

R⁵ is alkyl, (C1-C5) alkoxy, cyclo(C3-C8)alkyl, halo(C1-C5)alkyl, arylalkyl, alkynyl, aminoalkyl or mono- or di-alkylaminoalkyl.

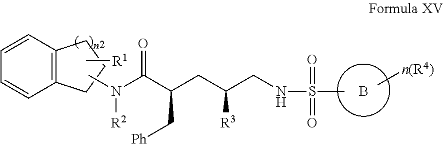

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Ring B is a 5- or 6-membered cyclic, heterocyclic, aryl or heteroaryl group containing 0, 1, 2 or 3 of C, O, N or S.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F;

n² is 1 or 2; and

n is 0, 1, 2 or 3.

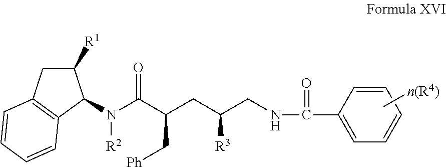

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F; and

n is 0, 1, 2 or 3.

Two R⁴ groups may be cyclized to form an infused ring.

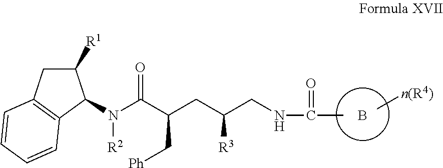

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Ring B is a 5- or 6-membered cyclic, heterocyclic or heteroaryl group containing 0, 1, 2 or 3 of C, O, N or S.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F; and

n is 0, 1, 2 or 3.

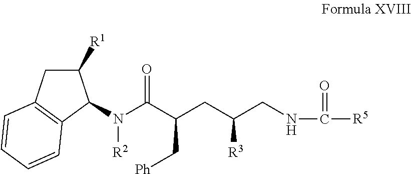

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

R⁵ is alkyl, (C1-C5) alkoxy, cyclo(C3-C8)alkyl, halo(C1-C5)alkyl, arylalkyl, alkynyl, aminoalkyl or mono- or di-alkylaminoalkyl.

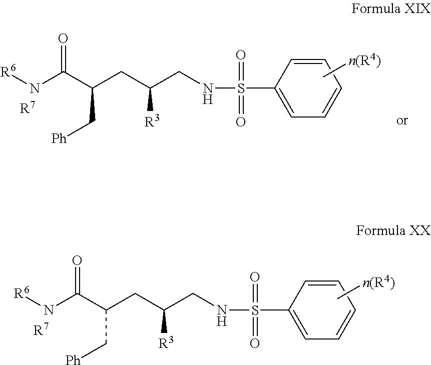

wherein

R⁶ and R⁷ are independently selected from the group consisting of H, hydroxy, alkynyl, (C1-C7)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl, cyclo(C3-C8)alkylamino, aryl, heteroaryl, arylamino, heteroarylamino, arylalkyl, haloarylalkyl, trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F; and n is 0, 1, 2 or 3.

R and R⁷ may be cyclized to form a ring.

R³ is H, hydroxy, methyl, alkoxy, oxo, acyloxy or halo. Ring B is a 5- or 6-membered cyclic, heterocyclic or heteroaryl group containing 0, 1, 2 or 3 of C, O, N or S.

Each R⁴ is independently selected from the group consisting of H, halo, cyano, hydroxy, nitro, alkenyl, alkynyl, (C1-C5)alkyl, halo(C1-CS)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F;

n is 0, 1, 2 or 3.

Two R⁴ groups may be cyclized to form an infused ring.

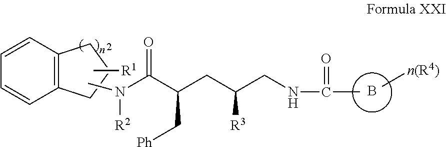

wherein:

R¹ is H, halo, cyano, alkyl, hydroxy, alkoxy, oxo or acyloxy.

R² is H or methyl.

R³ is H, hydroxy, methyl, alkoxy, oxo, acyloxy or halo.

Ring B is a 5- or 6-membered cyclic, heterocyclic, aryl or heteroaryl group containing 0 to 3 of C, O, N or S.

Each R⁴ is independently selected from the group consisting of H, halo, cyano, hydroxy, nitro, alkenyl, alkynyl, (C1-C5)alkyl, halo(C1-C5)alkyl, (C1-C5)alkoxy, halo(C1-C5)alkoxy, cyano(C1-C5)alkyl, amino, (C1-C5)alkylamino, di(C1-C5)alkylamino, amino(C1-C5)alkyl, (C1-C5)alkylamino(C1-C5)alkyl, di[(C1-C5)alkyl]amino(C1-C5)alkyl,trifluoromethylthio, hydroxy(C1-C5)alkyl, (C1-C5)alkoxy(C1-C5)alkyl, —C(O)R, —C(O)OH, —C(O)OR, —OC(O)R, —C(O)—NR₂, —CH₂C(O)R, —CH₂—C(O)OR, —CH₂—OC(O)R, —CH₂—C(O)—NR₂, S(O)₂R, S(O)₂N(R)₂, (C3-C8)cycloalkyl, and (C3-C8)cycloalkyl(C1-C5)alkyl, where R is alkyl, optionally substituted by one to three F;

n² is 1 or 2;

and n is 0, 1, 2 or 3.

In certain embodiments, a compound of the invention has one of general formulas XXII and XXIII as follows:

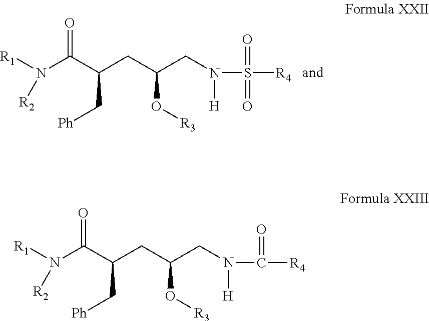

wherein:
R1, R2, R4: is a group of alkyl, aryl, heteroaryl, alkoxy, hydroxy, amino, alkylamino, diaklylamino, and acyl; and
R3 is H, alkyl, aryl or acyl group.

In certain embodiments, a compound of the invention has one of general formulas XXIV to XXVII as follows:

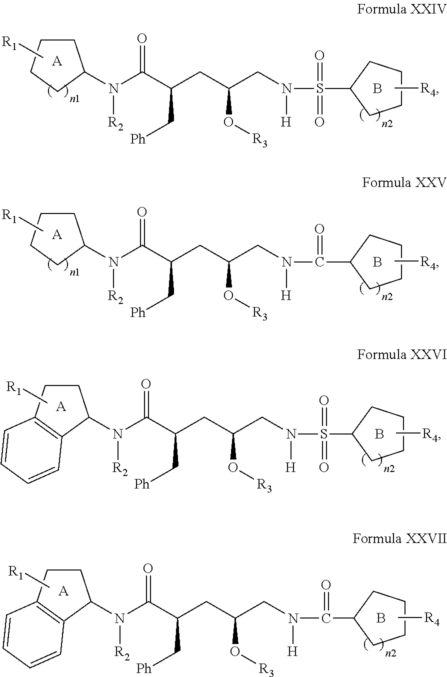

wherein:
Ring A: is a mono or multi-substituted aliphatic ring (n1=0, 1, 2), aromatic ring (n1=0, 1, 2) or aliphatic ring fused with another aliphatic or aromatic ring. Ring A may contains 1, 2 or 3 oxygen, nitrogen or sulfur atoms.
Ring B: is a substituted aliphatic ring (n1=0, 1, 2), aromatic ring (n1=0, 1, 2) or aliphatic ring fused with another aliphatic or aromatic ring. Ring A may contains 1, 2 or 3 oxygen, nitrogen or sulfur atoms.
R1 and R4 are each independently one or multi groups of alkyl, aryl, heteroaryl, alkoxy, hydroxy, amino, alkylamino, diaklylamino, acyl or halogen.
R2 is H, alkyl or aryl group
R3 is H, alkyl, aryl or acyl group,

In certain embodiments, the compounds of the application do not include the compounds described by Chu et al., Mol Pharmacol, 77:95-101, 2010.

In certain embodiments, the compounds of the application do not include the following compound:

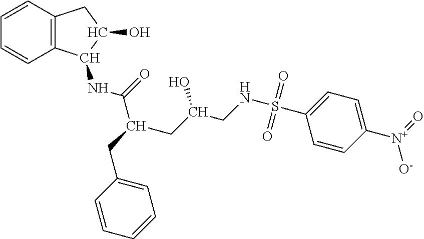 [SMILES: O=C(N[C@H]1c2ccccc2C[C@H]1O)[C@H](Cc1ccccc1)C[C@H](O)CNS(=O)(=O)c1ccc([N+](=O)[O-])cc1]

In certain embodiments, the compounds of the application do not include the following compound:

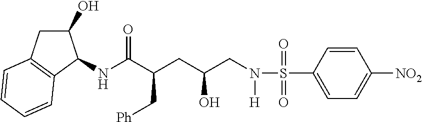 [SMILES: O=C(N[C@H]1c2ccccc2C[C@H]1O)[C@H](Cc1ccccc1)C[C@H](O)CNS(=O)(=O)c1ccc([N+](=O)[O-])cc1]

In certain embodiments, the compounds of the application do not include the following compound:

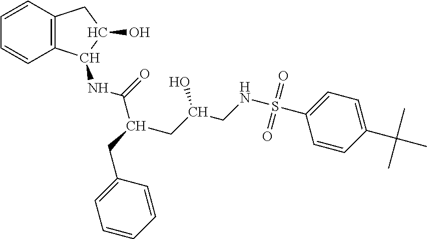 [SMILES: CC(C)(C)c1ccc(S(=O)(=O)NC[C@@H](O)C[C@@H](Cc2ccccc2)C(=O)N[C@H]2c3ccccc3C[C@H]2O)cc1]

In certain embodiments, the compounds of the application do not include the following compound:

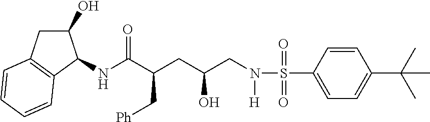 [SMILES: CC(C)(C)c1ccc(S(=O)(=O)NC[C@@H](O)C[C@@H](Cc2ccccc2)C(=O)N[C@H]2c3ccccc3C[C@H]2O)cc1]

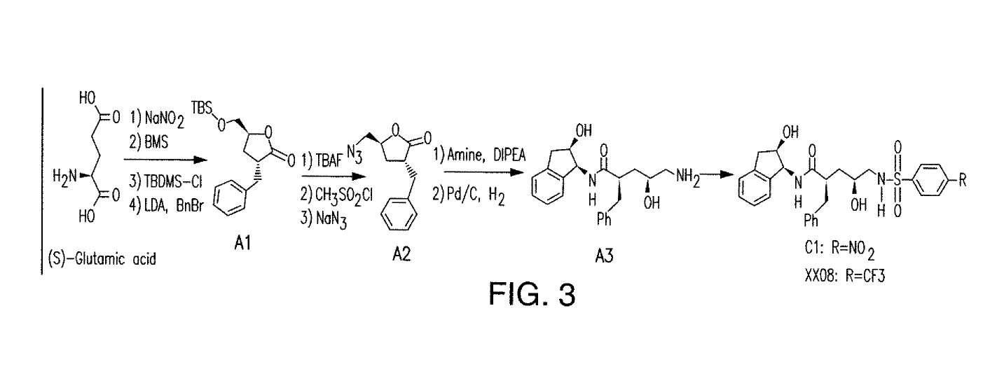

The foregoing compounds may be synthesized using a method analogous to that set forth below in scheme 1 (FIG. 3), depending on the functional groups/substituents utilized:

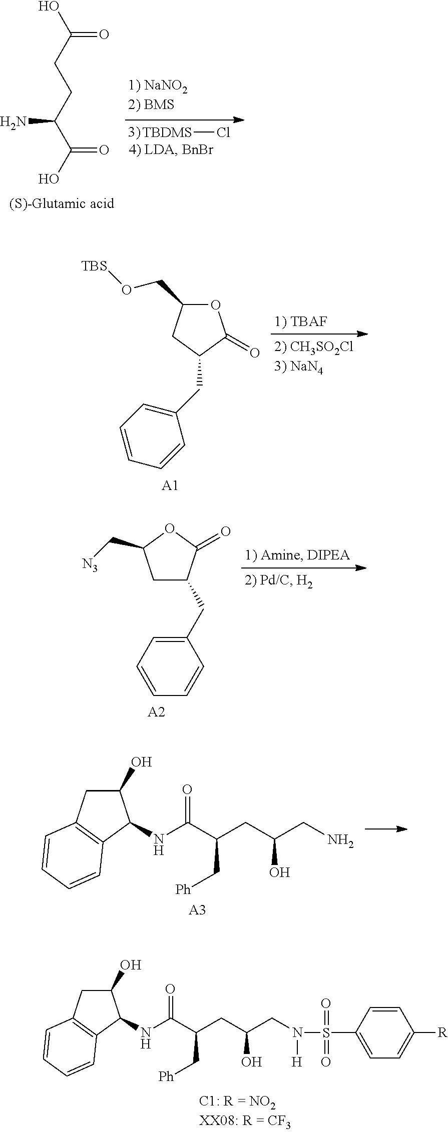

In certain, non-limiting embodiments, the compounds of the present application may be synthesized using a method analogous to that set forth below in scheme 2A, depending on the functional groups/substituents utilized:

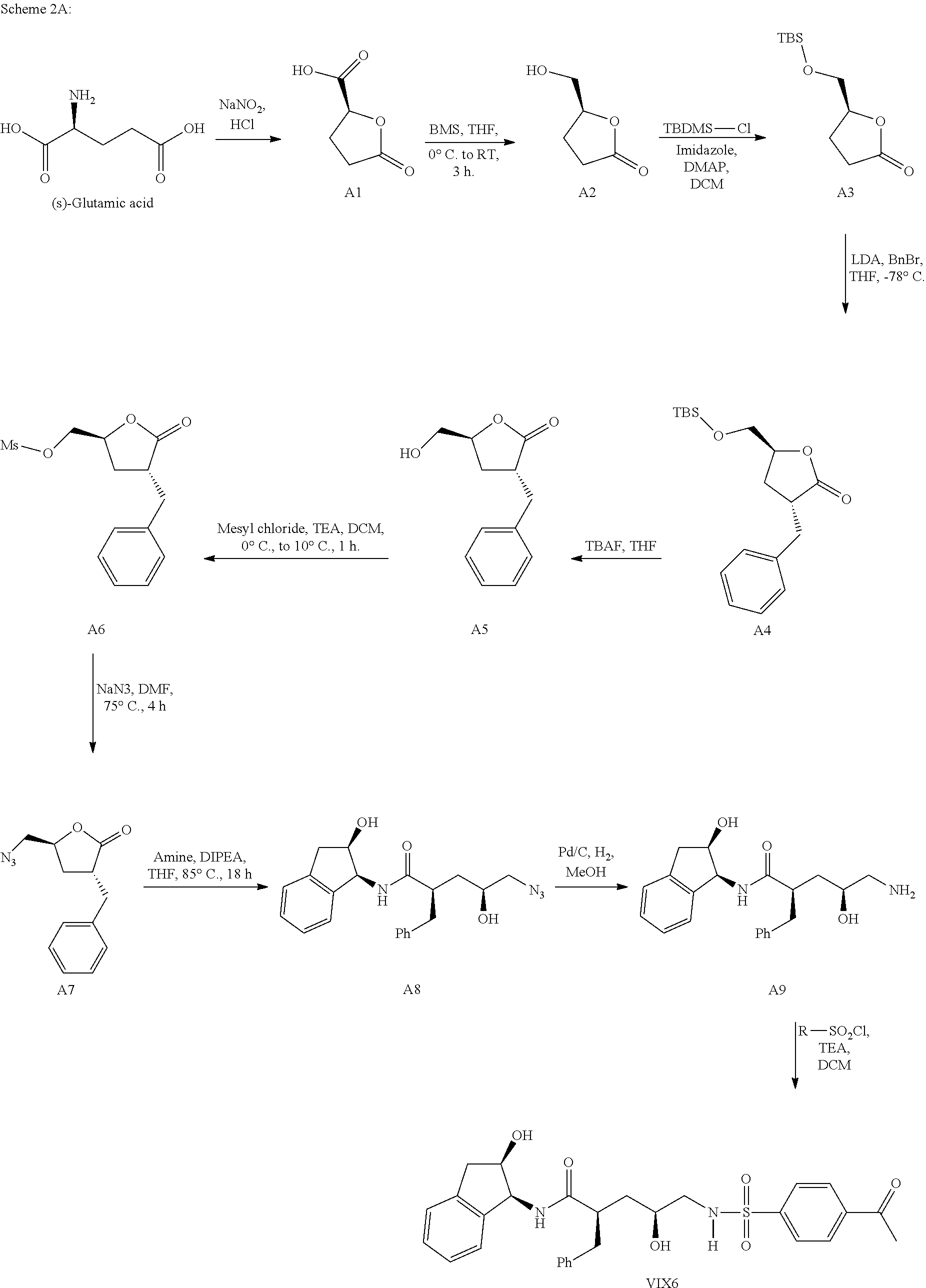

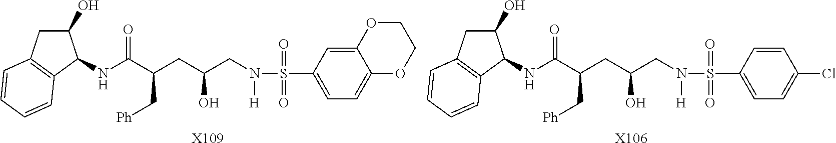

In certain, non-limiting embodiments, the compounds of the present application may be synthesized using a method analogous to that set forth below in scheme 2B, depending on the functional groups/substituents utilized:

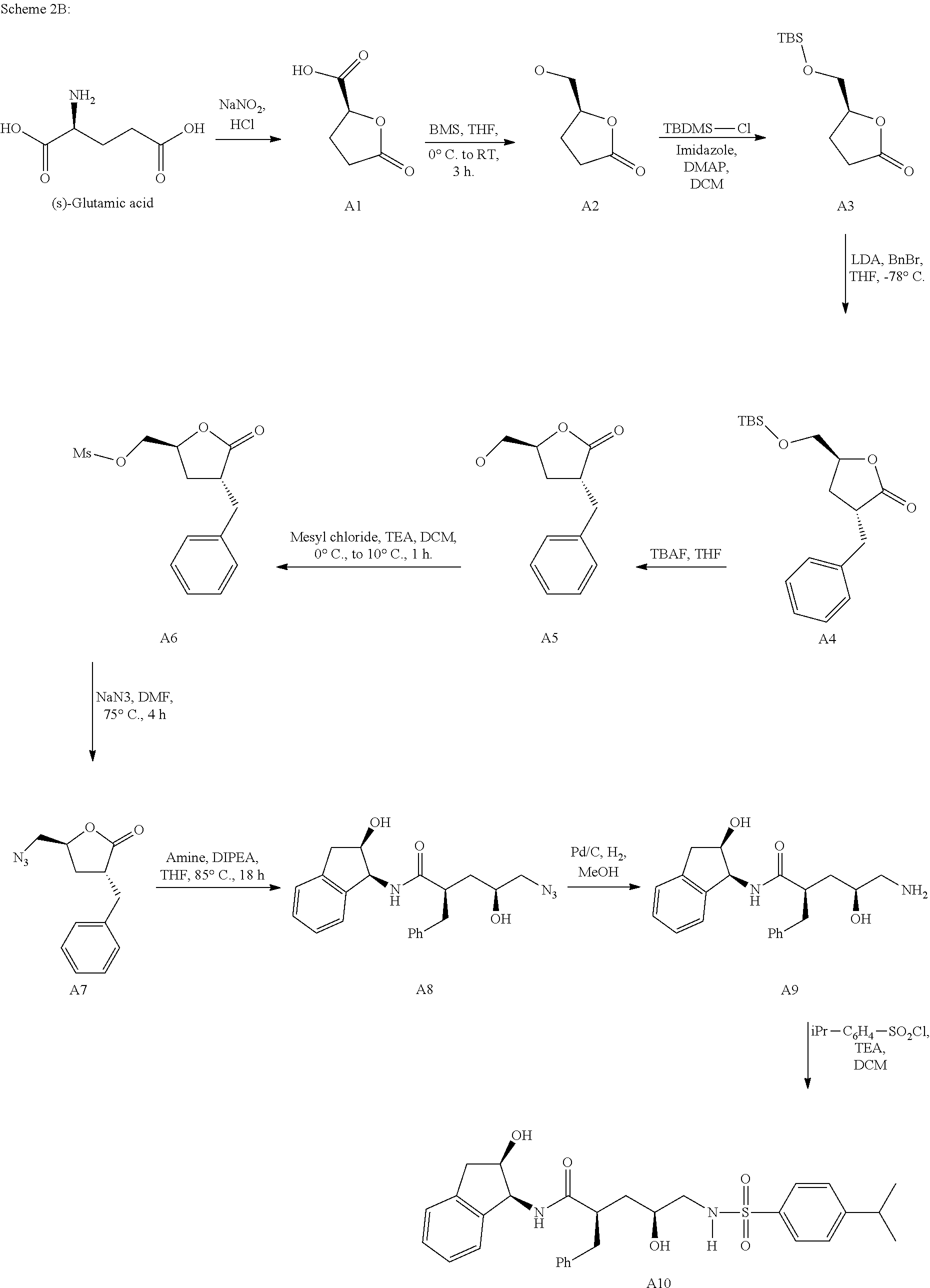

In certain, non-limiting embodiments, the compounds of the present application may be synthesized using a method analogous to that set forth below in scheme 2C, depending on the functional groups/substituents utilized:

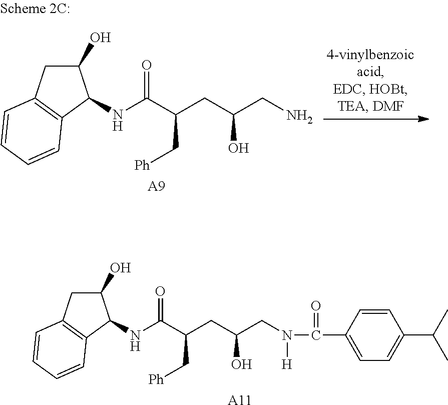

In certain, non-limiting embodiments, the compounds of the present application may be synthesized using a method analogous to that set forth below in scheme 2D, depending on the functional groups/substituents utilized:

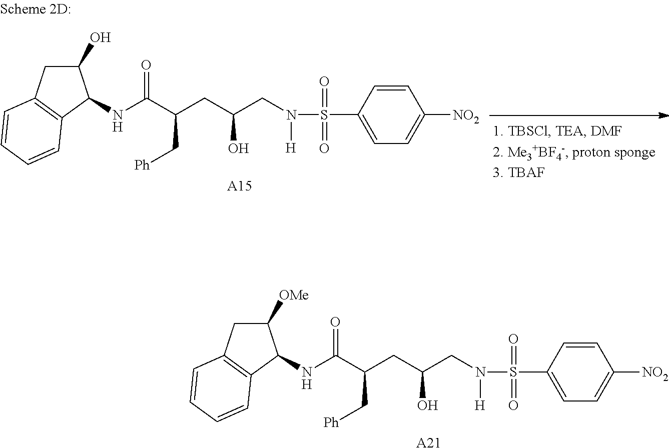

In certain, non-limiting embodiments, the compounds of the present application may be synthesized using a method analogous to that set forth below in scheme 3, depending on the functional groups/substituents utilized:

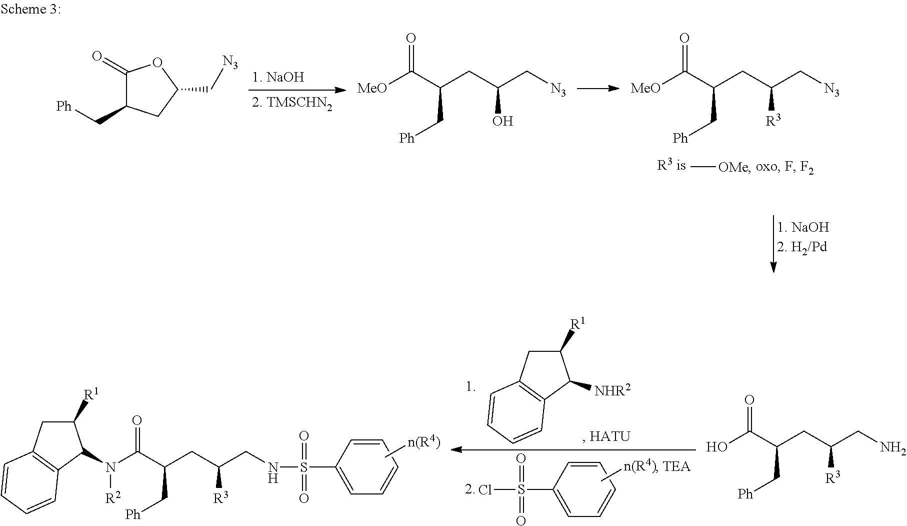

In certain, non-limiting embodiments, the compounds of the present application may be synthesized using a method analogous to that set forth below in scheme 4, depending on the functional groups/substituents utilized:

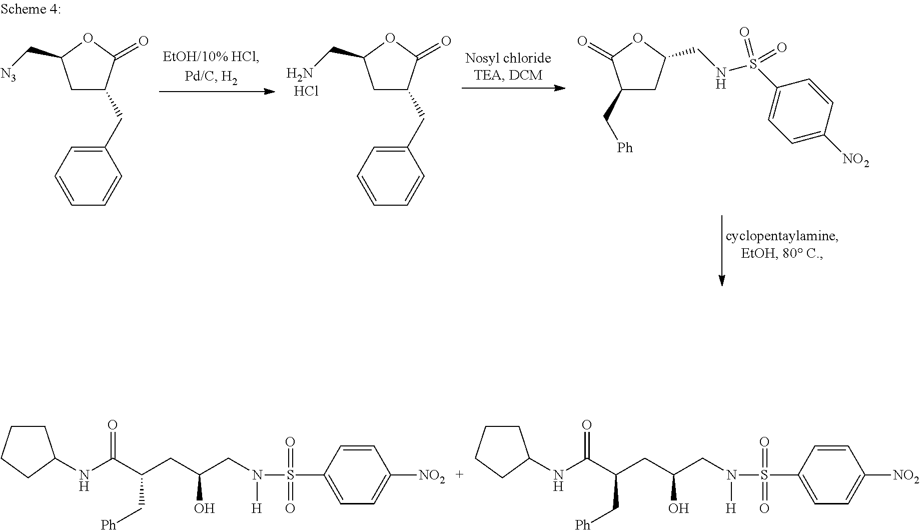

In certain, non-limiting embodiments, the compounds of the present application may be synthesized using a method analogous to that set forth below in scheme 5, depending on the functional groups/substituents utilized:

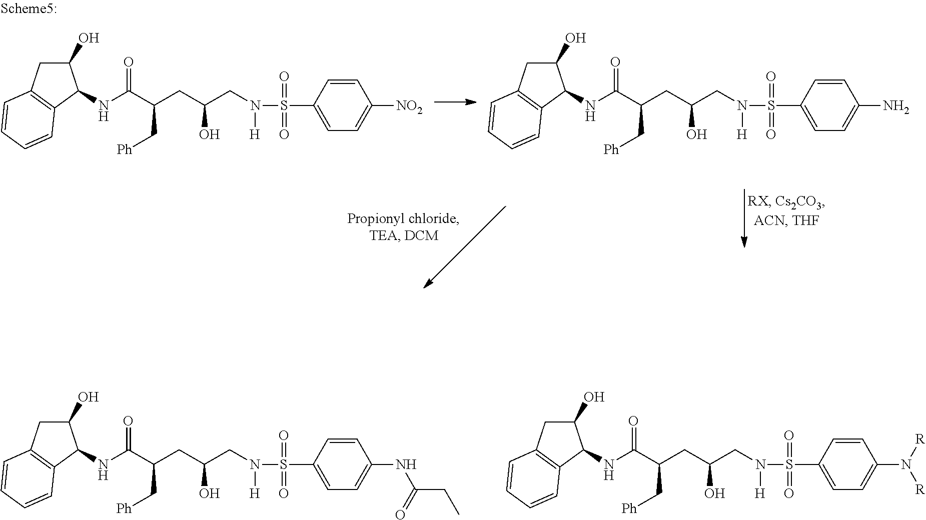

In certain, non-limiting embodiments, the compounds of the present application may be synthesized using a method analogous to that set forth below in scheme 6, depending on the functional groups/substituents utilized:

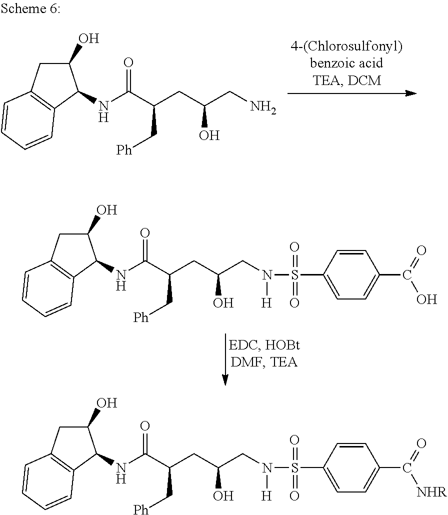

In certain, non-limiting embodiments, the compounds of the present application may be synthesized using a method analogous to that set forth below in scheme 7, depending on the functional groups/substituents utilized:

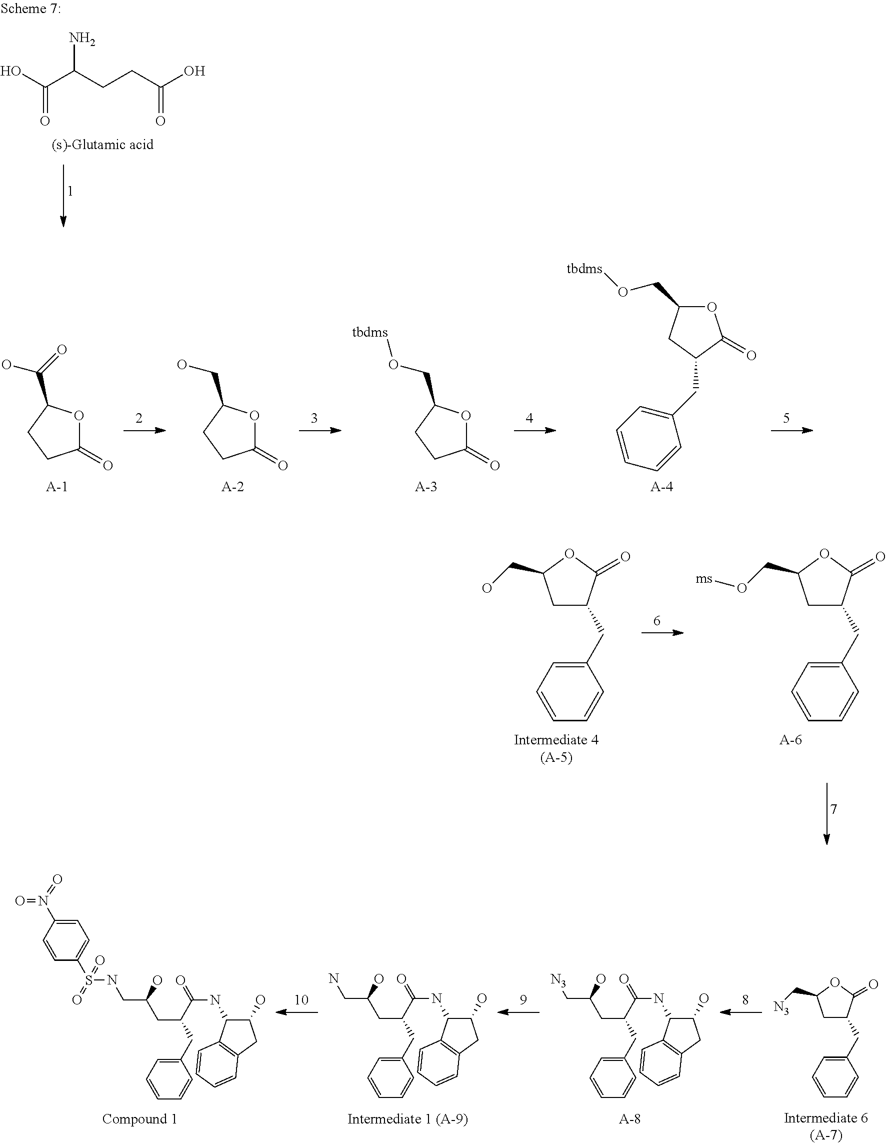

In certain non-limiting embodiments, a compound of the present application is synthesized according to the methods described in the present application, wherein an intermediate compound of the synthesis comprises one or more of the following compounds:

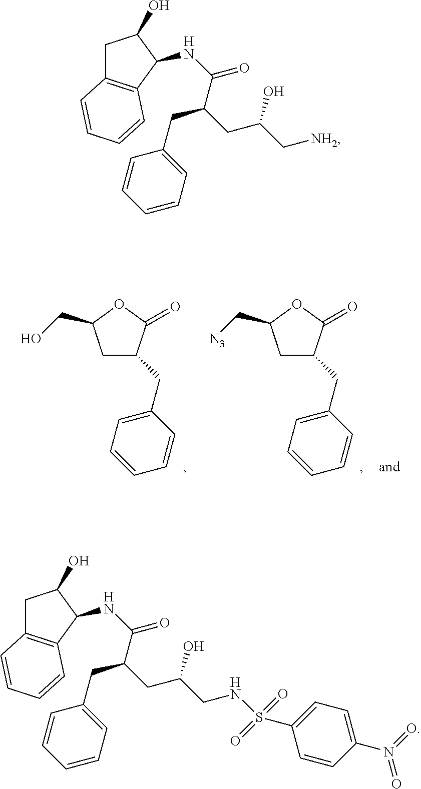 [SMILES: NC[C@@H](O)C[C@@H](Cc1ccccc1)C(=O)N[C@H]1c2ccccc2C[C@H]1O ; O=C1O[C@H](CO)C[C@H]1Cc1ccccc1 ; [N-]=[N+]=NC[C@@H]1C[C@@H](Cc2ccccc2)C(=O)O1 ; O=C(N[C@H]1c2ccccc2C[C@H]1O)[C@H](Cc1ccccc1)C[C@H](O)CNS(=O)(=O)c1ccc([N+](=O)[O-])cc1]

A compound of the invention is CNS accessible, meaning, functionally, that it can achieve therapeutic levels in the CNS after administration by one or more of oral, intramuscular, intradermnal, subcutaneous, intravenous, nasal, pulmonary, or rectal routes.

In particular non-limiting embodiments, compounds of the invention have a VIPR₂ inhibitory activity of at least 75 percent, or at least 80 percent, or at least 85 percent, or at least 90 percent, or at least 95 percent, or at least 100 percent, or at least 110 percent, or at least 120 percent, of the inhibitory activity of compound 1 of Chu et al., 2010, Molecular Pharmacol. 7795-101. For example, inhibitory activity may be determined using an assay system as described below.

In specific, non-limiting embodiments, a compound of the invention is a CNS accessible compound having fewer total nitrogen and oxygen atoms and/or which demonstrates, in a PAMPA or other published assay for BBB permeability, a superior permeability, relative to Compound 1 of Chu et al., 2010, Molecular Pharmacol. 7795-101.

A CNS accessible compound of the invention may, in certain non-limiting embodiments, have a molecular weight less than 600 or less than 570 or less than 560 or less than 550 or less than 540 or less than 530 or less than 520 or less than 510 or less than 500 or less than 450 Daltons. A CNS accessible compound of the invention may, in certain non-limiting embodiments, have a total polar surface area of less than 140 Å or less than 135 Å or less than 130 Å or less than 110 Å or less than 90 Å. In certain specific non-limiting embodiments, the total number of N or O atoms in a CNS accessible compound of the invention may be 9, less than 9, 8, less than 8, 7, less than 7, 6, less than 6, 5, less than 5, 4, less than 4, 3, less than 3, 2, less than 2, 1 or 0.

In non-limiting embodiments, a compound may be tested for agonist or antagonist activity at hERG and/or CYP3A4, where activity against one or both of these targets is desirably less than activity against VIPR₂, for example, the inhibitory activity against hERG and/or CYP3A4 is less than 80% of the inhibitory activity against VIPR₂, or the inhibitory activity against hERG and/or CYP3A4 is less than 70% of the inhibitory activity against VIPR₂, or the inhibitory activity against hERG and/or CYP3A4 is less than 60% of the inhibitory activity against VIPR₂, or the inhibitory activity against hERG and/or CYP3A4 is less than 50% of the inhibitory activity against VIPR₂, or the inhibitory activity against hERG and/or CYP3A4 is less than 40% of the inhibitory activity against VIPR₂, or the inhibitory activity against hERG and/or CYP3A4 is less than 30% of the inhibitory activity against VIPR₂, or the inhibitory activity against hERG and/or CYP3A4 is less than 20% of the inhibitory activity against VIPR₂, or the inhibitory activity against hERG and/or CYP3A4 is less than 10% of the inhibitory activity against VIPR₂, or the inhibitory activity against hERG and/or CYP3A4 is less than 1% of the inhibitory activity against VIPR₂, or the inhibitory activity against hERG and/or CYP3A4 is less than 0.1% of the inhibitory activity against VIPR₂.

In particular non-limiting embodiments, a compound of the invention has one or more of the following characteristics: IC₅₀<50 nM, hERG IC₅₀>30 μM, CYP3A4 IC₅₀>30 μM, log P 3-4, bioavailability (F %) 60%, t1/2>2 hr, brain-to-plasma distribution ratio>1.

In one specific, non-limiting embodiment, the invention provides for the compound

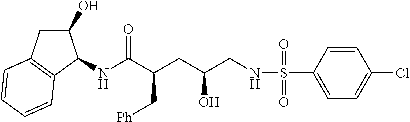 [SMILES: O=C(N[C@H]1c2ccccc2C[C@H]1O)[C@H](Cc1ccccc1)C[C@H](O)CNS(=O)(=O)c1ccc(Cl)cc1]

and for salts and chelates thereof. In certain embodiments, the invention provides for an enantiomer of said compound which differs in stereochemistry of at least one chiral center.

In one specific, non-limiting embodiment, the invention provides for the compound

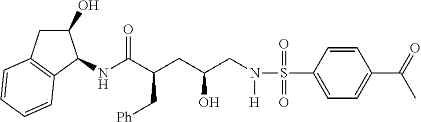 [SMILES: CC(=O)c1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

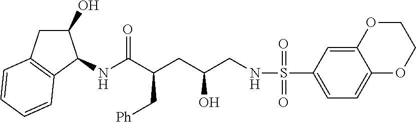 [SMILES: O=C(N[C@H]1c2ccccc2C[C@H]1O)[C@H](Cc1ccccc1)C[C@H](O)CNS(=O)(=O)c1ccc2c(c1)OCCO2]

and for salts and chelates thereof, In certain embodiments, the invention provides for an enantiomer of said compound which differs in stereochemistry of at least one chiral center.

In one specific, non-limiting embodiment, the invention provides for the compound

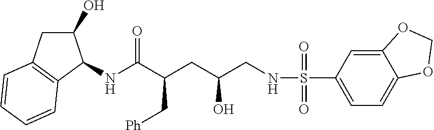 [SMILES: O=C(N[C@H]1c2ccccc2C[C@H]1O)[C@H](Cc1ccccc1)C[C@H](O)CNS(=O)(=O)c1ccc2c(c1)OCO2]

and for salts and chelates thereof. In certain embodiments, the invention provides for an enantiomer of said compound which differs in stereochemistry of at least one chiral center.

In one specific, non-limiting embodiment, the invention provides for the compound

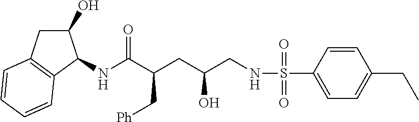 [SMILES: CCc1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

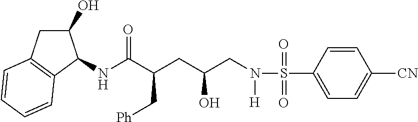 [SMILES: N#Cc1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

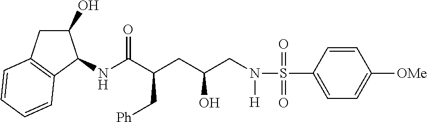 [SMILES: COc1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

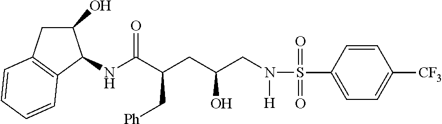 [SMILES: O=C(N[C@H]1c2ccccc2C[C@H]1O)[C@H](Cc1ccccc1)C[C@H](O)CNS(=O)(=O)c1ccc(C(F)(F)F)cc1]

and for salts and chelates thereof. In certain embodiments, the invention provides for an enantiomer of said compound which differs in stereochemistry of at least one chiral center.

In one specific, non-limiting embodiment, the invention provides for the compound

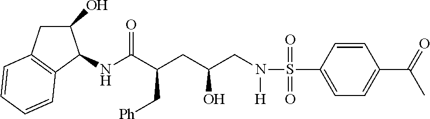 [SMILES: CC(=O)c1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

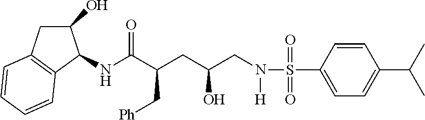 [SMILES: CC(C)c1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In one specific, non-limiting embodiment, the invention provides for the compound

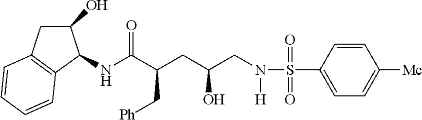 [SMILES: Cc1ccc(S(=O)(=O)NC[C@@H](O)C[C@@H](Cc2ccccc2)C(=O)N[C@H]2c3ccccc3C[C@H]2O)cc1]

and for salts and chelates thereof. In certain embodiments, the invention provides for an enantiomer of said compound which differs in stereochemistry of at least one chiral center.

In non-limiting embodiments the invention provides for compounds set forth in the following Tables 1, 2, 3 and 4 below (except that Ref C1 is a compound taught in Chu et al., supra, and is not a compound of the invention but is included for comparison purposes).

TABLE 1

| | | | | | | | Stability⁺ |
|---|---|---|---|---|---|---|---|
| | | | | | Lipo- | | 0 = Poor |
| | | | | tPSA | philicity | MW | 1 = Average |
| Name | Structure | IC50 | | Å | (cLogP) | (g/mol) | 2 = Good |
| Ref C1 | 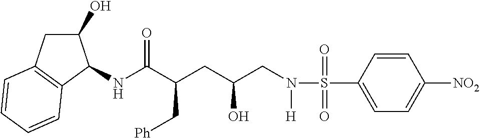 [SMILES: O=C(N[C@H]1c2ccccc2C[C@H]1O)[C@H](Cc1ccccc1)C[C@H](O)CNS(=O)(=O)c1ccc([N+](=O)[O-])cc1] | 1.3 | μM | 167 | 3.44 | 539.6 | 0 |
| K | 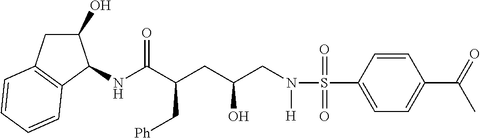 [SMILES: CC(=O)c1ccc(S(=O)(=O)NC[C@@H](O)C[C@@H](Cc2ccccc2)C(=O)N[C@H]2c3ccccc3C[C@H]2O)cc1] | 426 | nM | 132 | 3.14 | 536.64 | 0 |
| X106 | 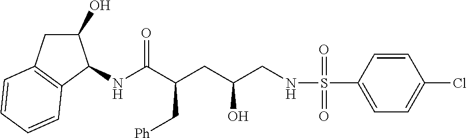 [SMILES: O=C(N[C@H]1c2ccccc2C[C@H]1O)[C@H](Cc1ccccc1)C[C@H](O)CNS(=O)(=O)c1ccc(Cl)cc1] | 280 | nM | 115 | 4.07 | 529.05 | 2 |
| X109 | 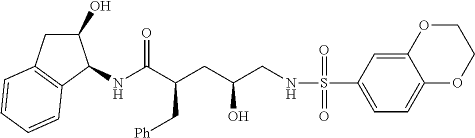 [SMILES: O=C(N[C@H]1c2ccccc2C[C@H]1O)[C@H](Cc1ccccc1)C[C@H](O)CNS(=O)(=O)c1ccc2c(c1)OCCO2] | 206 | nM | 134 | 3.3 | 552.64 | 1 |
| X110 | 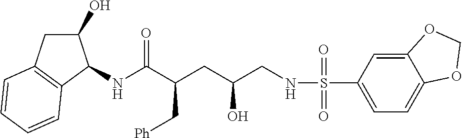 [SMILES: O=C(N[C@H]1c2ccccc2C[C@H]1O)[C@H](Cc1ccccc1)C[C@H](O)CNS(=O)(=O)c1ccc2c(c1)OCO2] | 2.16 | μM | 134 | 3.34 | 538.61 | 1 |
| X115 | 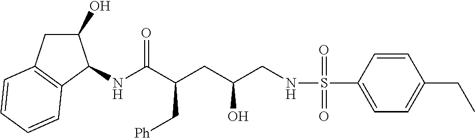 [SMILES: CCc1ccc(S(=O)(=O)NC[C@@H](O)C[C@@H](Cc2ccccc2)C(=O)N[C@H]2c3ccccc3C[C@H]2O)cc1] | 1.13 | μM | 115 | 4.08 | 522.66 | 2 |
| C | 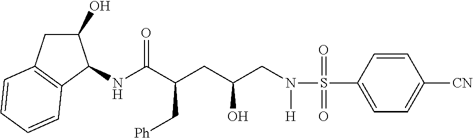 [SMILES: N#Cc1ccc(S(=O)(=O)NC[C@@H](O)C[C@@H](Cc2ccccc2)C(=O)N[C@H]2c3ccccc3C[C@H]2O)cc1] | 1.33 | μM | 139 | 3.19 | 519.61 | 1.5 |
| E | 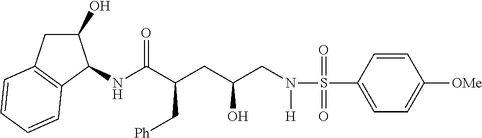 [SMILES: COc1ccc(S(=O)(=O)NC[C@@H](O)C[C@@H](Cc2ccccc2)C(=O)N[C@H]2c3ccccc3C[C@H]2O)cc1] | 1.02 | μM | 124 | 3.33 | 524.63 | 1 |
| I | 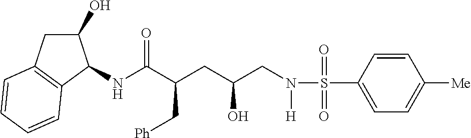 [SMILES: Cc1ccc(S(=O)(=O)NC[C@@H](O)C[C@@H](Cc2ccccc2)C(=O)N[C@H]2c3ccccc3C[C@H]2O)cc1] | 1.18 | μM | 115 | 3.55 | 508.63 | 2 |

⁺Expected stability.

TABLE 2

| | | % Activity | | Lipo- | |
|---|---|---|---|---|---|
| | | (and BRET | tPSA | philicity | MW |
| Name | Structure | results) | Å | (cLogP) | (g/mol) |
| Ref C1 | 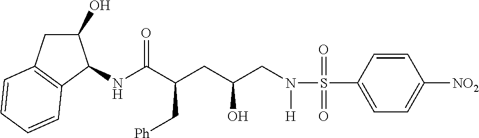 [SMILES: O=C(N[C@H]1c2ccccc2C[C@H]1O)[C@H](Cc1ccccc1)C[C@H](O)CNS(=O)(=O)c1ccc([N+](=O)[O-])cc1] | 84.51667 | 167 | 3.44 | 539.6 |
| C | 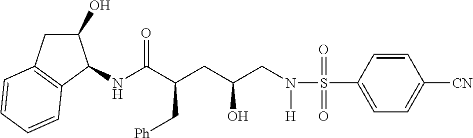 [SMILES: N#Cc1ccc(S(=O)(=O)NC[C@@H](O)C[C@@H](Cc2ccccc2)C(=O)N[C@H]2c3ccccc3C[C@H]2O)cc1] | 91.75000 BRET 1.33 μM | 139.5 | 2.49 | 519.61 |
| E | 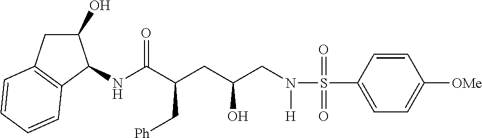 [SMILES: COc1ccc(S(=O)(=O)NC[C@@H](O)C[C@@H](Cc2ccccc2)C(=O)N[C@H]2c3ccccc3C[C@H]2O)cc1] | 91.60000 BRET 1.02 μM | 125.0 | 2.98 | 524.63 |
| S | 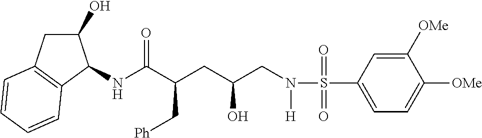 [SMILES: COc1ccc(S(=O)(=O)NC[C@@H](O)C[C@@H](Cc2ccccc2)C(=O)N[C@H]2c3ccccc3C[C@H]2O)cc1OC] | 30.10000 | 134.2 | 2.71 | 554.65 |
| F | 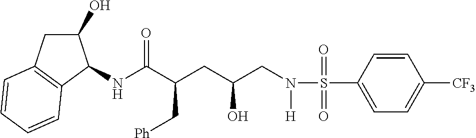 [SMILES: O=C(N[C@H]1c2ccccc2C[C@H]1O)[C@H](Cc1ccccc1)C[C@H](O)CNS(=O)(=O)c1ccc(C(F)(F)F)cc1] | 95.70000 BRET 2.34 μM | 115.7 | 3.94 | 562.60 |
| R | 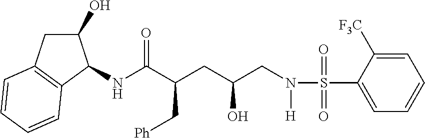 | 22.85000 | 115.7 | 3.94 | 562.60 |
| K | 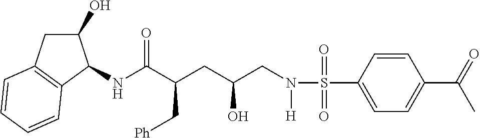 [SMILES: CC(=O)c1ccc(S(=O)(=O)NC[C@@H](O)C[C@@H](Cc2ccccc2)C(=O)N[C@H]2c3ccccc3C[C@H]2O)cc1] | 92.35000 BRET- 426 nM | 132.8 | 2.50 | 536.64 |
| T | 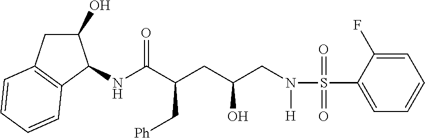 [SMILES: O=C(N[C@H]1c2ccccc2C[C@H]1O)[C@H](Cc1ccccc1)C[C@H](O)CNS(=O)(=O)c1ccccc1F] | 40.15000 | 115.7 | 3.20 | 512.59 |
| A3 | 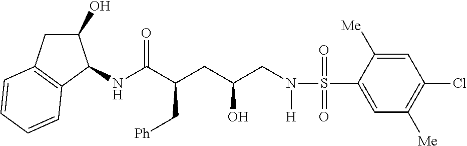 [SMILES: Cc1cc(S(=O)(=O)NC[C@@H](O)C[C@@H](Cc2ccccc2)C(=O)N[C@H]2c3ccccc3C[C@H]2O)c(C)cc1Cl] | 20.85000 | 115.7 | 4.77 | 557.10 |
| A10 | 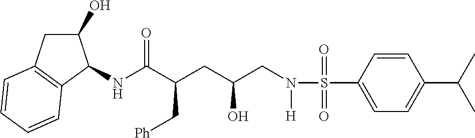 [SMILES: CC(C)c1ccc(S(=O)(=O)NC[C@@H](O)C[C@@H](Cc2ccccc2)C(=O)N[C@H]2c3ccccc3C[C@H]2O)cc1] | 92.95000 BRET 3.44 μM | 115.7 | 4.48 | 536.68 |
| I | 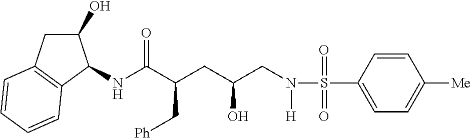 [SMILES: Cc1ccc(S(=O)(=O)NC[C@@H](O)C[C@@H](Cc2ccccc2)C(=O)N[C@H]2c3ccccc3C[C@H]2O)cc1] | 94.10000 BRET 1.98 μM | 115.7 | 3.55 | 508.63 |
| N | 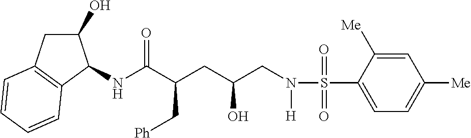 [SMILES: Cc1ccc(S(=O)(=O)NC[C@@H](O)C[C@@H](Cc2ccccc2)C(=O)N[C@H]2c3ccccc3C[C@H]2O)c(C)c1] | 27.55000 | 115.7 | 4.05 | 522.66 |
| J | 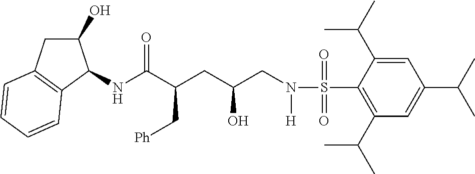 [SMILES: CC(C)c1cc(C(C)C)c(S(=O)(=O)NC[C@@H](O)C[C@@H](Cc2ccccc2)C(=O)N[C@H]2c3ccccc3C[C@H]2O)c(C(C)C)c1] | 49.15000 | 115.7 | 7.34 | 620.84 |
| L | 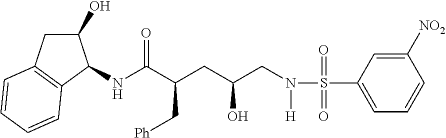 [SMILES: O=C(N[C@H]1c2ccccc2C[C@H]1O)[C@H](Cc1ccccc1)C[C@H](O)CNS(=O)(=O)c1cccc([N+](=O)[O-])c1] | 27.35000 | 167.5 | 2.80 | 539.60 |
| P | 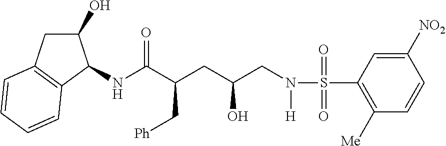 [SMILES: Cc1ccc([N+](=O)[O-])cc1S(=O)(=O)NC[C@@H](O)C[C@@H](Cc1ccccc1)C(=O)N[C@H]1c2ccccc2C[C@H]1O] | 8.85000 | 167.5 | 3.30 | 553.63 |
| A11 | 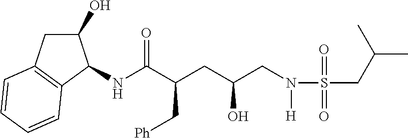 [SMILES: CC(C)CS(=O)(=O)NC[C@@H](O)C[C@@H](Cc1ccccc1)C(=O)N[C@H]1c2ccccc2C[C@H]1O] | 21.10000 | 115.7 | 3.13 | 474.61 |
| A5 | 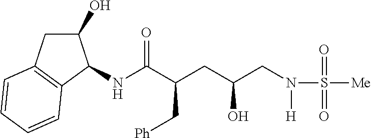 [SMILES: CS(=O)(=O)NC[C@@H](O)C[C@@H](Cc1ccccc1)C(=O)N[C@H]1c2ccccc2C[C@H]1O] | 20.95000 | 115.7 | 1.67 | 432.53 |
| B2A | 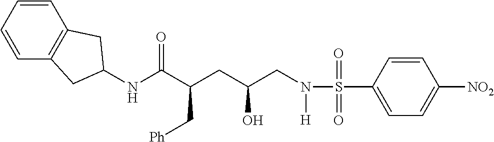 [SMILES: O=C(NC1Cc2ccccc2C1)[C@H](Cc1ccccc1)C[C@H](O)CNS(=O)(=O)c1ccc([N+](=O)[O-])cc1] | 12.90000 | 147.3 | 3.36 | 523.60 |
| B13A | 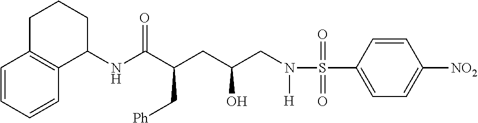 [SMILES: O=C(NC1CCCc2ccccc21)[C@H](Cc1ccccc1)C[C@H](O)CNS(=O)(=O)c1ccc([N+](=O)[O-])cc1] | 13.85000 | 147.3 | 4.28 | 537.63 |
| B3A | 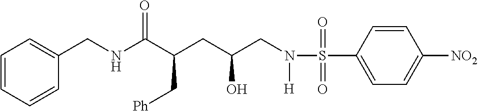 [SMILES: O=C(NCc1ccccc1)[C@H](Cc1ccccc1)C[C@H](O)CNS(=O)(=O)c1ccc([N+](=O)[O-])cc1] | 10.65000 | 147.3 | 3.31 | 497.56 |
| B3B | 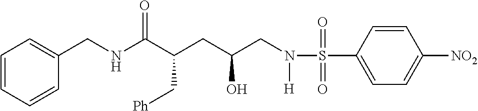 [SMILES: O=C(NCc1ccccc1)[C@@H](Cc1ccccc1)C[C@H](O)CNS(=O)(=O)c1ccc([N+](=O)[O-])cc1] | 6.05000 | 147.3 | 3.31 | 497.56 |
| B4A | 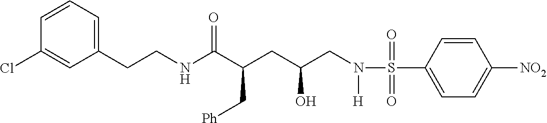 [SMILES: O=C(NCCc1cccc(Cl)c1)[C@H](Cc1ccccc1)C[C@H](O)CNS(=O)(=O)c1ccc([N+](=O)[O-])cc1] | −0.40000 | 147.3 | 4.19 | 546.04 |
| B4B | 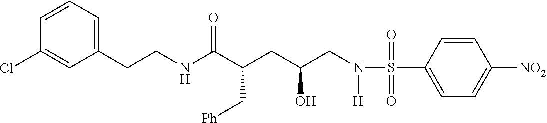 [SMILES: O=C(NCCc1cccc(Cl)c1)[C@@H](Cc1ccccc1)C[C@H](O)CNS(=O)(=O)c1ccc([N+](=O)[O-])cc1] | 13.85000 | 147.3 | 4.19 | 546.04 |
| B1A | 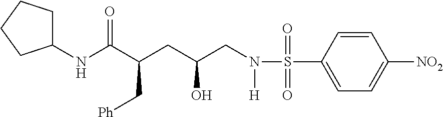 [SMILES: O=C(NC1CCCC1)[C@H](Cc1ccccc1)C[C@H](O)CNS(=O)(=O)c1ccc([N+](=O)[O-])cc1] | 6.95000 | 147.3 | 2.85 | 475.56 |
| B1B | 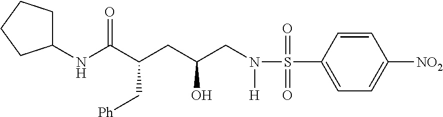 [SMILES: O=C(NC1CCCC1)[C@@H](Cc1ccccc1)C[C@H](O)CNS(=O)(=O)c1ccc([N+](=O)[O-])cc1] | 0.50000 | 147.3 | 2.85 | 475.56 |
| B5A | 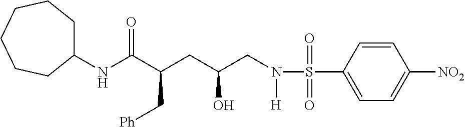 [SMILES: O=C(NC1CCCCCC1)[C@H](Cc1ccccc1)C[C@H](O)CNS(=O)(=O)c1ccc([N+](=O)[O-])cc1] | 5.25000 | 147.3 | 3.97 | 503.61 |
| B5B | 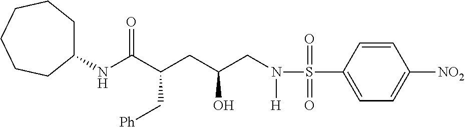 | 10.55000 | 147.3 | 3.97 | 503.61 |
| B6A | 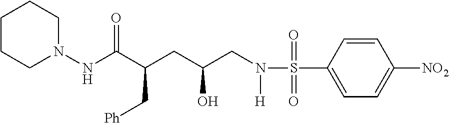 [SMILES: O=C(NN1CCCCC1)[C@H](Cc1ccccc1)C[C@H](O)CNS(=O)(=O)c1ccc([N+](=O)[O-])cc1] | 5.45000 | 150.6 | 2.45 | 490.57 |
| B6B | 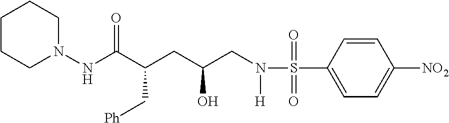 [SMILES: O=C(NN1CCCCC1)[C@@H](Cc1ccccc1)C[C@H](O)CNS(=O)(=O)c1ccc([N+](=O)[O-])cc1] | 5.35000 | 150.6 | 2.45 | 490.57 |
| B8A | 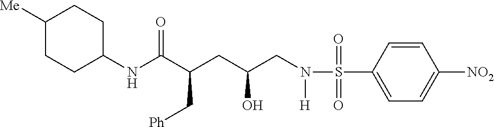 [SMILES: CC1CCC(NC(=O)[C@H](Cc2ccccc2)C[C@H](O)CNS(=O)(=O)c2ccc([N+](=O)[O-])cc2)CC1] | −1.20000 | 147.3 | 3.93 | 503.61 |
| B8B | 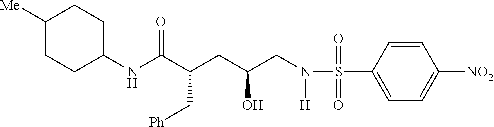 [SMILES: CC1CCC(NC(=O)[C@@H](Cc2ccccc2)C[C@H](O)CNS(=O)(=O)c2ccc([N+](=O)[O-])cc2)CC1] | 2.15000 | 147.3 | 3.93 | 503.61 |
| B9B | 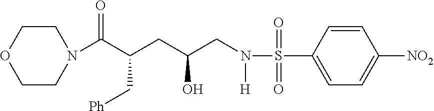 [SMILES: O=C([C@@H](Cc1ccccc1)C[C@H](O)CNS(=O)(=O)c1ccc([N+](=O)[O-])cc1)N1CCOCC1] | 1.65000 | 147.8 | 1.82 | 477.53 |
| B9A | 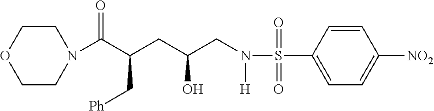 [SMILES: O=C([C@H](Cc1ccccc1)C[C@H](O)CNS(=O)(=O)c1ccc([N+](=O)[O-])cc1)N1CCOCC1] | 0.60000 | 147.8 | 1.82 | 477.53 |
| B18B | 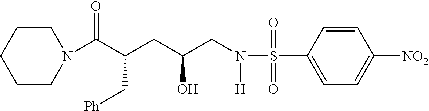 [SMILES: O=C([C@@H](Cc1ccccc1)C[C@H](O)CNS(=O)(=O)c1ccc([N+](=O)[O-])cc1)N1CCCCC1] | 12.15000 BRET IC-50 NC* | 138.5 | 2.61 | 475.56 |
| B18A | 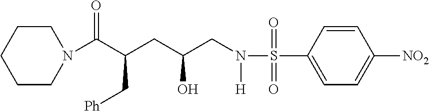 [SMILES: O=C([C@H](Cc1ccccc1)C[C@H](O)CNS(=O)(=O)c1ccc([N+](=O)[O-])cc1)N1CCCCC1] | −1.15000 BRET IC50 NC* | 138.5 | 2.61 | 475.56 |
| B19 | 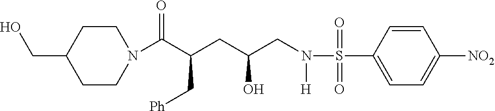 [SMILES: O=C([C@H](Cc1ccccc1)C[C@H](O)CNS(=O)(=O)c1ccc([N+](=O)[O-])cc1)N1CCC(CO)CC1] | 0.85000 | 158.8 | 1.14 | 505.58 |
| B16A | 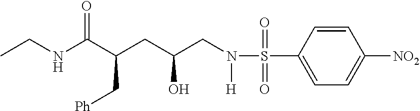 [SMILES: CCNC(=O)[C@H](Cc1ccccc1)C[C@H](O)CNS(=O)(=O)c1ccc([N+](=O)[O-])cc1] | −9.25000 BRET IC50-NC* | 147.3 | 1.90 | 435.49 |
| B16B | 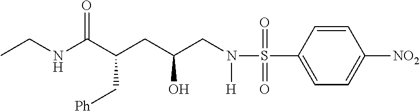 [SMILES: CCNC(=O)[C@@H](Cc1ccccc1)C[C@H](O)CNS(=O)(=O)c1ccc([N+](=O)[O-])cc1] | −14.90000 | 147.3 | 1.90 | 435.49 |
| B15A | 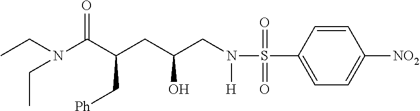 [SMILES: CCN(CC)C(=O)[C@H](Cc1ccccc1)C[C@H](O)CNS(=O)(=O)c1ccc([N+](=O)[O-])cc1] | 0.70000 | 138.5 | 2.81 | 463.55 |
| B15B | 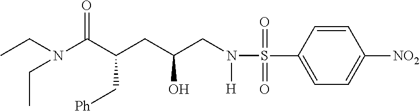 [SMILES: CCN(CC)C(=O)[C@@H](Cc1ccccc1)C[C@H](O)CNS(=O)(=O)c1ccc([N+](=O)[O-])cc1] | −6.60000 | 138.5 | 2.81 | 463.55 |
| B21A | 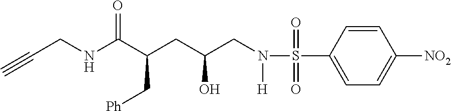 [SMILES: C#CCNC(=O)[C@H](Cc1ccccc1)C[C@H](O)CNS(=O)(=O)c1ccc([N+](=O)[O-])cc1] | 3.55000 | 147.3 | 1.98 | 445.49 |
| B21B | 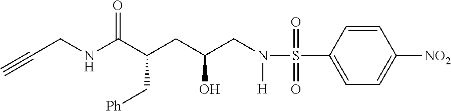 [SMILES: C#CCNC(=O)[C@@H](Cc1ccccc1)C[C@H](O)CNS(=O)(=O)c1ccc([N+](=O)[O-])cc1] | 10.25000 | 147.3 | 1.98 | 445.49 |
| B7A | 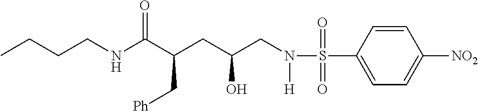 [SMILES: CCCCNC(=O)[C@H](Cc1ccccc1)C[C@H](O)CNS(=O)(=O)c1ccc([N+](=O)[O-])cc1] | 9.55000 | 147.3 | 2.96 | 463.55 |
| B7B | 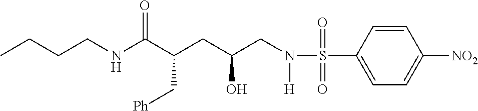 [SMILES: CCCCNC(=O)[C@@H](Cc1ccccc1)C[C@H](O)CNS(=O)(=O)c1ccc([N+](=O)[O-])cc1] | 13.45000 BRET- 1.8 mM | 147.3 | 2.96 | 463.55 |
| B10A | 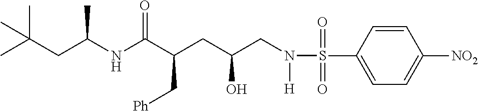 [SMILES: C[C@H](CC(C)(C)C)NC(=O)[C@H](Cc1ccccc1)C[C@H](O)CNS(=O)(=O)c1ccc([N+](=O)[O-])cc1] | −5.20000 BRET IC50-NC* | 147.3 | 4.07 | 505.63 |
| B10B | 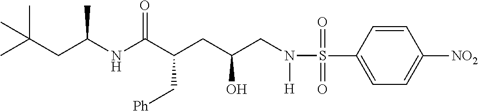 [SMILES: C[C@H](CC(C)(C)C)NC(=O)[C@@H](Cc1ccccc1)C[C@H](O)CNS(=O)(=O)c1ccc([N+](=O)[O-])cc1] | 13.8000 BRET IC50-NC* | 147.3 | 4.07 | 505.63 |
| B20 | 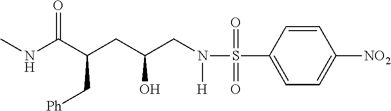 [SMILES: CNC(=O)[C@H](Cc1ccccc1)C[C@H](O)CNS(=O)(=O)c1ccc([N+](=O)[O-])cc1] | 6.25000 | 147.3 | 1.38 | 421.47 |
| B12A | 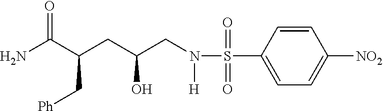 [SMILES: NC(=O)[C@H](Cc1ccccc1)C[C@H](O)CNS(=O)(=O)c1ccc([N+](=O)[O-])cc1] | −10.45000 | 161.3 | 1.20 | 407.44 |
| B12B | 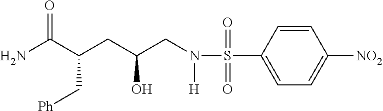 [SMILES: NC(=O)[C@@H](Cc1ccccc1)C[C@H](O)CNS(=O)(=O)c1ccc([N+](=O)[O-])cc1] | 8.35000 | 161.3 | 1.20 | 407.44 |
| A24 | 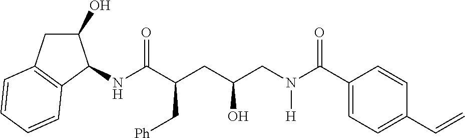 [SMILES: C=Cc1ccc(C(=O)NC[C@@H](O)C[C@@H](Cc2ccccc2)C(=O)N[C@H]2c3ccccc3C[C@H]2O)cc1] | 12.80000 | 98.7 | 4.07 | 484.59 |
| A25 | 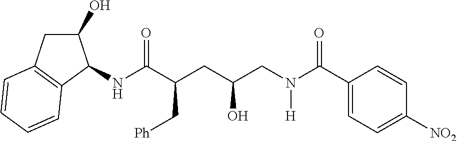 [SMILES: O=C(NC[C@@H](O)C[C@@H](Cc1ccccc1)C(=O)N[C@H]1c2ccccc2C[C@H]1O)c1ccc([N+](=O)[O-])cc1] | 18.75000 | 105.4 | 3.09 | 503.55 |
| Q | 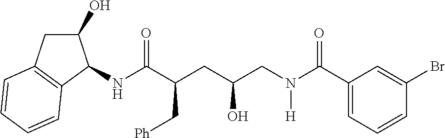 [SMILES: O=C(NC[C@@H](O)C[C@@H](Cc1ccccc1)C(=O)N[C@H]1c2ccccc2C[C@H]1O)c1cccc(Br)c1] | 12.75000 | 98.7 | 4.21 | 537.44 |
| O | 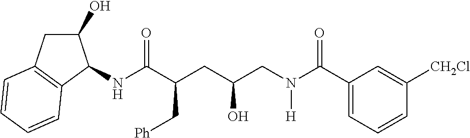 | 2.40000 | 98.7 | 3.91 | 507.02 |
| G | 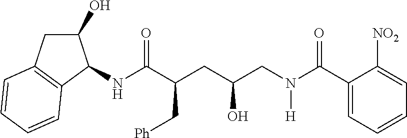 [SMILES: O=C(NC[C@@H](O)C[C@@H](Cc1ccccc1)C(=O)N[C@H]1c2ccccc2C[C@H]1O)c1ccccc1[N+](=O)[O-]] | 17.60000 | 150.7 | 3.09 | 503.55 |
| H | 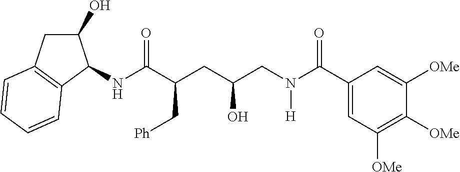 [SMILES: COc1cc(C(=O)NC[C@@H](O)C[C@@H](Cc2ccccc2)C(=O)N[C@H]2c3ccccc3C[C@H]2O)cc(OC)c1OC] | 13.65000 | 126.4 | 2.65 | 548.63 |
| M | 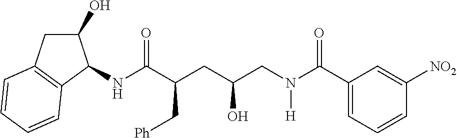 [SMILES: O=C(NC[C@@H](O)C[C@@H](Cc1ccccc1)C(=O)N[C@H]1c2ccccc2C[C@H]1O)c1cccc([N+](=O)[O-])c1] | 4.75000 | 150.5 | 3.09 | 503.55 |
| A26 | 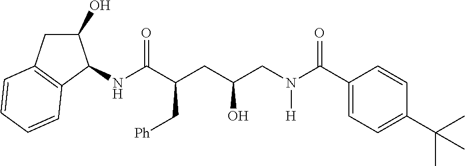 [SMILES: CC(C)(C)c1ccc(C(=O)NC[C@@H](O)C[C@@H](Cc2ccccc2)C(=O)N[C@H]2c3ccccc3C[C@H]2O)cc1] | 15.50000 | 98.7 | 5.18 | 514.66 |
| B | 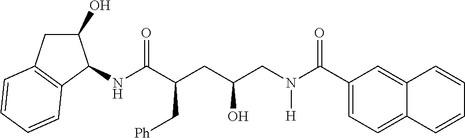 [SMILES: O=C(NC[C@@H](O)C[C@@H](Cc1ccccc1)C(=O)N[C@H]1c2ccccc2C[C@H]1O)c1ccc2ccccc2c1] | 16.60000 | 98.7 | 4.52 | 508.61 |
| D | 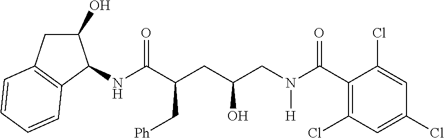 [SMILES: O=C(NC[C@@H](O)C[C@@H](Cc1ccccc1)C(=O)N[C@H]1c2ccccc2C[C@H]1O)c1c(Cl)cc(Cl)cc1Cl] | 21.90000 | 98.7 | 5.49 | 561.88 |
| A2 | 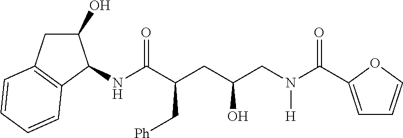 [SMILES: O=C(NC[C@@H](O)C[C@@H](Cc1ccccc1)C(=O)N[C@H]1c2ccccc2C[C@H]1O)c1ccco1] | 26.20000 | 107.9 | 2.53 | 448.51 |
| A16 | 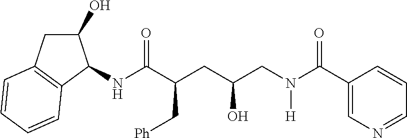 [SMILES: O=C(NC[C@@H](O)C[C@@H](Cc1ccccc1)C(=O)N[C@H]1c2ccccc2C[C@H]1O)c1cccnc1] | 5.05000 | 111.0 | 1.85 | 459.54 |
| A17 | 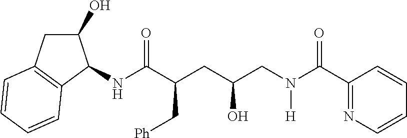 [SMILES: O=C(NC[C@@H](O)C[C@@H](Cc1ccccc1)C(=O)N[C@H]1c2ccccc2C[C@H]1O)c1ccccn1] | 12.90000 | 111.0 | 1.85 | 459.584 |
| A18 | 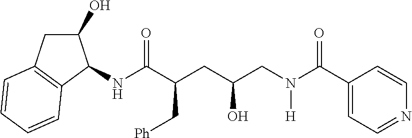 [SMILES: O=C(NC[C@@H](O)C[C@@H](Cc1ccccc1)C(=O)N[C@H]1c2ccccc2C[C@H]1O)c1ccncc1] | 6.10000 | 111.0 | 1.85 | 459.54 |
| A13 | 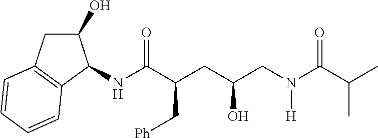 [SMILES: CC(C)C(=O)NC[C@@H](O)C[C@@H](Cc1ccccc1)C(=O)N[C@H]1c2ccccc2C[C@H]1O] | −8.60000 | 98.7 | 2.86 | 424.53 |
| A1 | 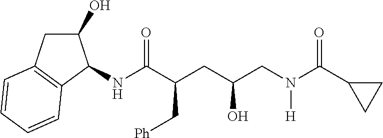 [SMILES: O=C(NC[C@@H](O)C[C@@H](Cc1ccccc1)C(=O)N[C@H]1c2ccccc2C[C@H]1O)C1CC1] | 26.55000 | 98.7 | 2.60 | 422.52 |
| A7 | 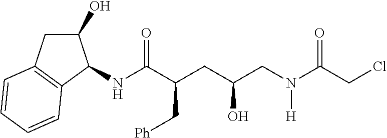 [SMILES: O=C(CCl)NC[C@@H](O)C[C@@H](Cc1ccccc1)C(=O)N[C@H]1c2ccccc2C[C@H]1O] | 16.55000 | 98.7 | 2.13 | 430.92 |
| A22 | 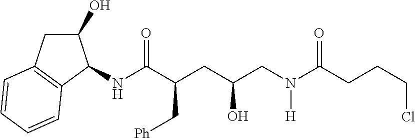 [SMILES: O=C(CCCCl)NC[C@@H](O)C[C@@H](Cc1ccccc1)C(=O)N[C@H]1c2ccccc2C[C@H]1O] | 18.50000 | 98.7 | 2.94 | 458.98 |
| A19 | 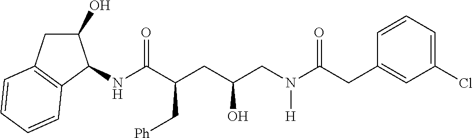 [SMILES: O=C(Cc1cccc(Cl)c1)NC[C@@H](O)C[C@@H](Cc1ccccc1)C(=O)N[C@H]1c2ccccc2C[C@H]1O] | 21.85000 | 98.7 | 4.45 | 507.02 |
| A8 | 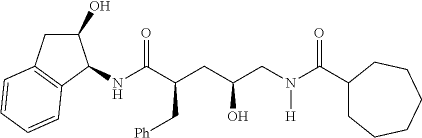 [SMILES: O=C(NC[C@@H](O)C[C@@H](Cc1ccccc1)C(=O)N[C@H]1c2ccccc2C[C@H]1O)C1CCCCCC1] | 13.75000 | 98.7 | 4.61 | 478.62 |
| A21 | 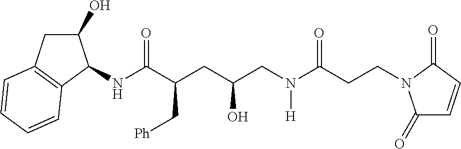 [SMILES: O=C(CCN1C(=O)C=CC1=O)NC[C@@H](O)C[C@@H](Cc1ccccc1)C(=O)N[C@H]1c2ccccc2C[C@H]1O] | 7.75000 | 136.0 | 1.91 | 505.56 |
| A6 | 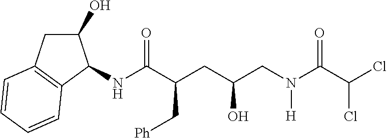 [SMILES: O=C(NC[C@@H](O)C[C@@H](Cc1ccccc1)C(=O)N[C@H]1c2ccccc2C[C@H]1O)C(Cl)Cl] | 19.80000 | 98.7 | 3.19 | 465.37 |
| A23 | 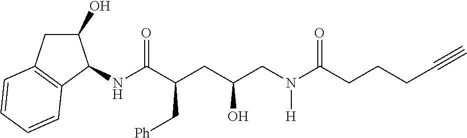 [SMILES: C#CCCCC(=O)NC[C@@H](O)C[C@@H](Cc1ccccc1)C(=O)N[C@H]1c2ccccc2C[C@H]1O] | 26.20000 | 98.7 | 2.93 | 448.55 |
| A15 | 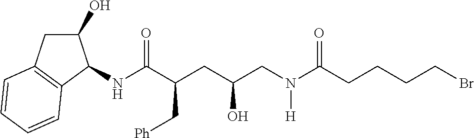 [SMILES: O=C(CCCCBr)NC[C@@H](O)C[C@@H](Cc1ccccc1)C(=O)N[C@H]1c2ccccc2C[C@H]1O] | −0.60000 | 98.7 | 3.46 | 517.46 |
| X081 | 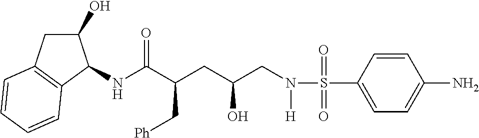 [SMILES: Nc1ccc(S(=O)(=O)NC[C@@H](O)C[C@@H](Cc2ccccc2)C(=O)N[C@H]2c3ccccc3C[C@H]2O)cc1] | 20.00000 | 141.8 | 1.83 | 509.62 |
| X082 | 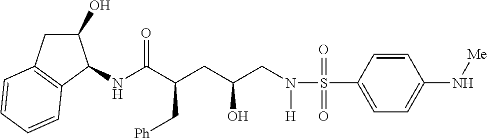 [SMILES: CNc1ccc(S(=O)(=O)NC[C@@H](O)C[C@@H](Cc2ccccc2)C(=O)N[C@H]2c3ccccc3C[C@H]2O)cc1] | −1.50000 | 127.8 | 2.56 | 523.64 |
| X083 | 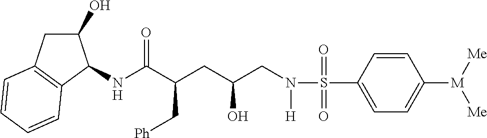 | 2.65000 | 119.0 | 3.22 | 537.67 |
| X084 | 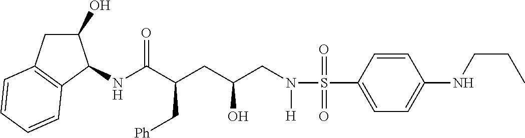 [SMILES: CCCNc1ccc(S(=O)(=O)NC[C@@H](O)C[C@@H](Cc2ccccc2)C(=O)N[C@H]2c3ccccc3C[C@H]2O)cc1] | 15.60000 | 127.8 | 3.61 | 551.70 |
| X085 | 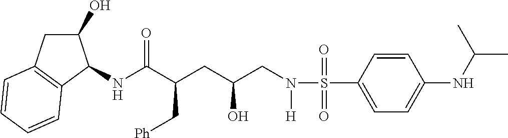 [SMILES: CC(C)Nc1ccc(S(=O)(=O)NC[C@@H](O)C[C@@H](Cc2ccccc2)C(=O)N[C@H]2c3ccccc3C[C@H]2O)cc1] | 16.20000 | 127.8 | 3.39 | 551.70 |
| X086 | 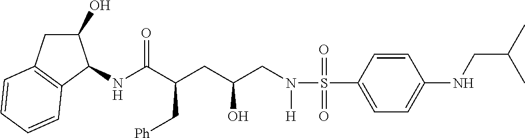 [SMILES: CC(C)CNc1ccc(S(=O)(=O)NC[C@@H](O)C[C@@H](Cc2ccccc2)C(=O)N[C@H]2c3ccccc3C[C@H]2O)cc1] | 19.65000 | 127.8 | 4.01 | 565.75 |
| X087 | 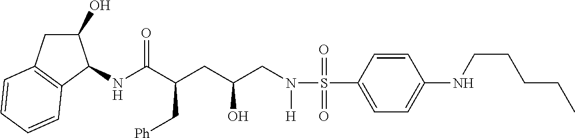 [SMILES: CCCCCNc1ccc(S(=O)(=O)NC[C@@H](O)C[C@@H](Cc2ccccc2)C(=O)N[C@H]2c3ccccc3C[C@H]2O)cc1] | 15.20000 | 127.8 | 4.67 | 579.75 |
| X088 | 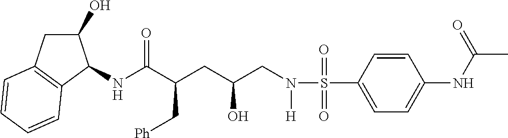 [SMILES: CC(=O)Nc1ccc(S(=O)(=O)NC[C@@H](O)C[C@@H](Cc2ccccc2)C(=O)N[C@H]2c3ccccc3C[C@H]2O)cc1] | 11.65000 | 144.8 | 2.08 | 551.65 |
| X089 | 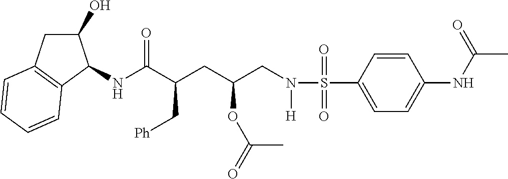 [SMILES: CC(=O)Nc1ccc(S(=O)(=O)NC[C@H](C[C@@H](Cc2ccccc2)C(=O)N[C@H]2c3ccccc3C[C@H]2O)OC(C)=O)cc1] | 6.75000 | 150.9 | 2.93 | 593.69 |
| X090 | 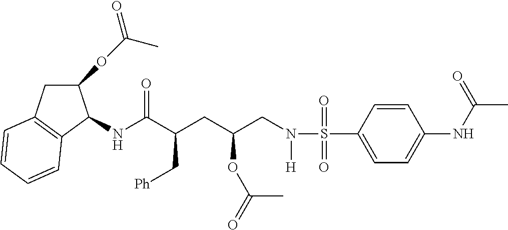 [SMILES: CC(=O)Nc1ccc(S(=O)(=O)NC[C@H](C[C@@H](Cc2ccccc2)C(=O)N[C@H]2c3ccccc3C[C@H]2OC(C)=O)OC(C)=O)cc1] | 1.95000 | 157.0 | 3.83 | 635.73 |
| X091 | 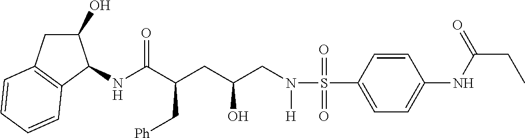 [SMILES: CCC(=O)Nc1ccc(S(=O)(=O)NC[C@@H](O)C[C@@H](Cc2ccccc2)C(=O)N[C@H]2c3ccccc3C[C@H]2O)cc1] | 8.25000 | 144.8 | 2.60 | 565.68 |
| X092 | 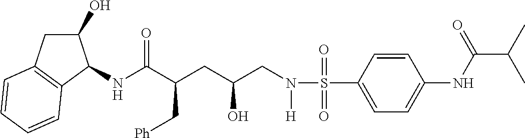 [SMILES: CC(C)C(=O)Nc1ccc(S(=O)(=O)NC[C@@H](O)C[C@@H](Cc2ccccc2)C(=O)N[C@H]2c3ccccc3C[C@H]2O)cc1] | 4.60000 | 144.8 | 2.91 | 579.71 |
| X093 | 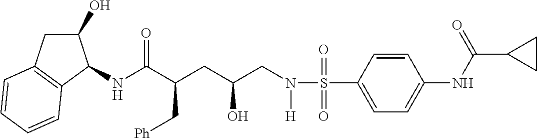 [SMILES: O=C(Nc1ccc(S(=O)(=O)NC[C@@H](O)C[C@@H](Cc2ccccc2)C(=O)N[C@H]2c3ccccc3C[C@H]2O)cc1)C1CC1] | 15.05000 | 144.8 | 2.66 | 577.69 |
| X094 | 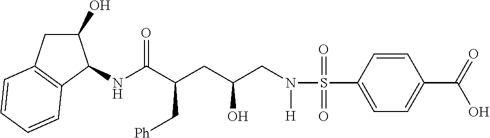 [SMILES: O=C(O)c1ccc(S(=O)(=O)NC[C@@H](O)C[C@@H](Cc2ccccc2)C(=O)N[C@H]2c3ccccc3C[C@H]2O)cc1] | 13.10000 | 153.0 | 2.80 | 538.61 |
| X095 | 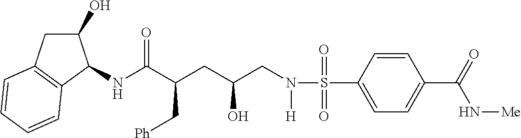 [SMILES: CNC(=O)c1ccc(S(=O)(=O)NC[C@@H](O)C[C@@H](Cc2ccccc2)C(=O)N[C@H]2c3ccccc3C[C@H]2O)cc1] | 34.25000 | 144.8 | 1.78 | 551.65 |
| X096 | 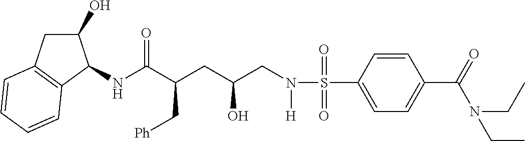 [SMILES: CCN(CC)C(=O)c1ccc(S(=O)(=O)NC[C@@H](O)C[C@@H](Cc2ccccc2)C(=O)N[C@H]2c3ccccc3C[C@H]2O)cc1] | 15.30000 | 136.0 | 2.58 | 593.73 |
| X097 | 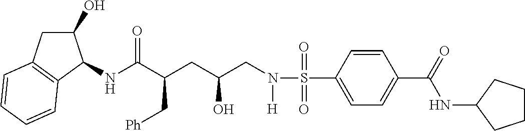 [SMILES: O=C(NC1CCCC1)c1ccc(S(=O)(=O)NC[C@@H](O)C[C@@H](Cc2ccccc2)C(=O)N[C@H]2c3ccccc3C[C@H]2O)cc1] | 15.95000 | 144.8 | 3.25 | 605.74 |
| X098 | 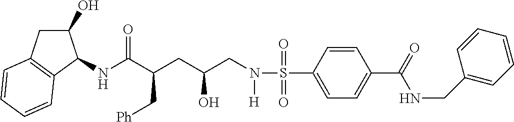 [SMILES: O=C(NCc1ccccc1)c1ccc(S(=O)(=O)NC[C@@H](O)C[C@@H](Cc2ccccc2)C(=O)N[C@H]2c3ccccc3C[C@H]2O)cc1] | 41.40000 | 144.8 | 3.74 | 627.75 |
| X099 | 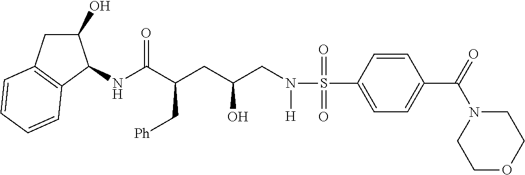 [SMILES: O=C(N[C@H]1c2ccccc2C[C@H]1O)[C@H](Cc1ccccc1)C[C@H](O)CNS(=O)(=O)c1ccc(C(=O)N2CCOCC2)cc1] | 10.05000 | 145.3 | 1.69 | 607.72 |
| X100 | 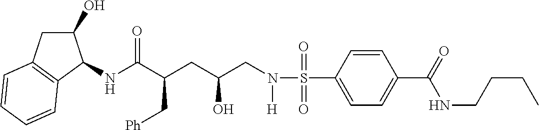 [SMILES: CCCCNC(=O)c1ccc(S(=O)(=O)NC[C@@H](O)C[C@@H](Cc2ccccc2)C(=O)N[C@H]2c3ccccc3C[C@H]2O)cc1] | 7.55000 | 144.8 | 3.36 | 593.73 |
| X101 | 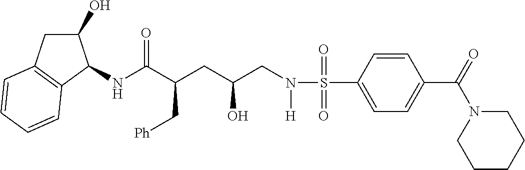 [SMILES: O=C(N[C@H]1c2ccccc2C[C@H]1O)[C@H](Cc1ccccc1)C[C@H](O)CNS(=O)(=O)c1ccc(C(=O)N2CCCCC2)cc1] | −6.85000 | 136.0 | 2.71 | 605.74 |
| X102 | 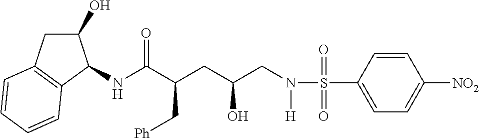 [SMILES: O=C(N[C@H]1c2ccccc2C[C@H]1O)[C@H](Cc1ccccc1)C[C@H](O)CNS(=O)(=O)c1ccc([N+](=O)[O-])cc1] | −2.05000 | 156.5 | 3.56 | 553.63 |
| X103 | 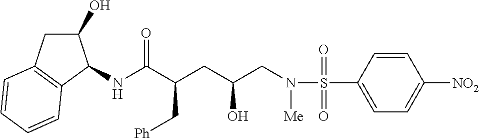 [SMILES: CN(C[C@@H](O)C[C@@H](Cc1ccccc1)C(=O)N[C@H]1c2ccccc2C[C@H]1O)S(=O)(=O)c1ccc([N+](=O)[O-])cc1] | 6.90000 | 147.8 | 4.06 | 567.65 |
| A28 | 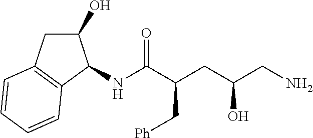 [SMILES: NC[C@@H](O)C[C@@H](Cc1ccccc1)C(=O)N[C@H]1c2ccccc2C[C@H]1O] | 17.450000 | 95.6 | 1.41 | 354.44 |
| B13B | 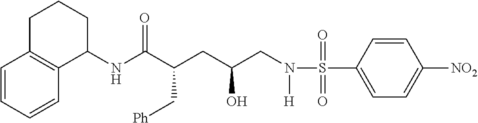 [SMILES: O=C(NC1CCCc2ccccc21)[C@@H](Cc1ccccc1)C[C@H](O)CNS(=O)(=O)c1ccc([N+](=O)[O-])cc1] | 9.0000 | 147.3 | 4.28 | 537.63 |
| B14A | 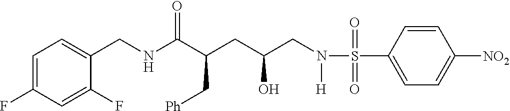 [SMILES: O=C(NCc1ccc(F)cc1F)[C@H](Cc1ccccc1)C[C@H](O)CNS(=O)(=O)c1ccc([N+](=O)[O-])cc1] | 27.2000 | 147.31 | 3.60 | 147.31 |
| B14B | 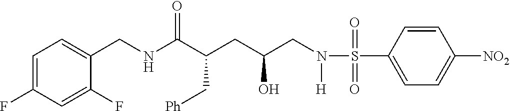 [SMILES: O=C(NCc1ccc(F)cc1F)[C@@H](Cc1ccccc1)C[C@H](O)CNS(=O)(=O)c1ccc([N+](=O)[O-])cc1] | 21.1000 | 147.31 | 3.60 | 533.54 |

*NC indicates that there was No Change in BRET ratio (<0.1) in response to treatment with the compound in the BRET assay.

TABLE 3

| | | | | | Lipo- | |
|---|---|---|---|---|---|---|
| | | IC50 | | tPSA | philicity | MW |
| Name | Structure | (from BRET) | | Å | (cLogP) | (g/mol) |
| X105 | 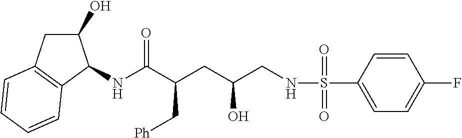 [SMILES: O=C(N[C@H]1c2ccccc2C[C@H]1O)[C@H](Cc1ccccc1)C[C@H](O)CNS(=O)(=O)c1ccc(F)cc1] | 6.03 | μM | 115.7 | 3.50 | 512.59 |
| X106 | 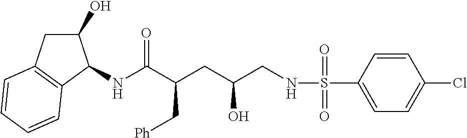 [SMILES: O=C(N[C@H]1c2ccccc2C[C@H]1O)[C@H](Cc1ccccc1)C[C@H](O)CNS(=O)(=O)c1ccc(Cl)cc1] | 2.02 | μM | 115.7 | 4.07 | 529.05 |
| X107 | 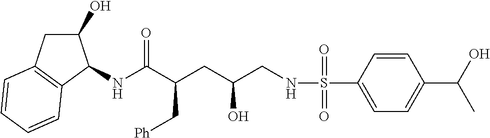 [SMILES: CC(O)c1ccc(S(=O)(=O)NC[C@@H](O)C[C@@H](Cc2ccccc2)C(=O)N[C@H]2c3ccccc3C[C@H]2O)cc1] | 40.6 | μM | 136.0 | 2.32 | 538.66 |
| X108 | 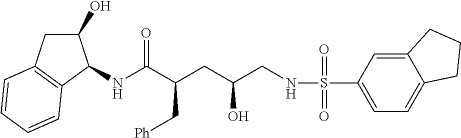 [SMILES: O=C(N[C@H]1c2ccccc2C[C@H]1O)[C@H](Cc1ccccc1)C[C@H](O)CNS(=O)(=O)c1ccc2c(c1)CCC2] | 10.0 | μM | 115.7 | 4.06 | 534.67 |
| X109 | 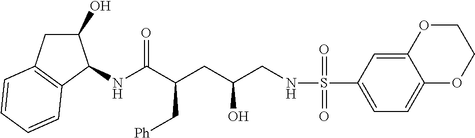 [SMILES: O=C(N[C@H]1c2ccccc2C[C@H]1O)[C@H](Cc1ccccc1)C[C@H](O)CNS(=O)(=O)c1ccc2c(c1)OCCO2] | 33.7 | μM | 134.2 | 3.30 | 552.64 |
| X110 | 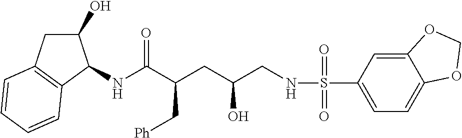 [SMILES: O=C(N[C@H]1c2ccccc2C[C@H]1O)[C@H](Cc1ccccc1)C[C@H](O)CNS(=O)(=O)c1ccc2c(c1)OCO2] | 3.81 | μM | 134.2 | 3.34 | 538.61 |
| X111 | 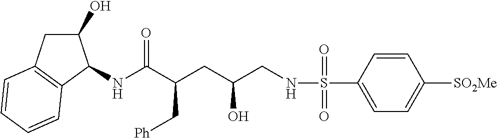 [SMILES: CS(=O)(=O)c1ccc(S(=O)(=O)NC[C@@H](O)C[C@@H](Cc2ccccc2)C(=O)N[C@H]2c3ccccc3C[C@H]2O)cc1] | 13.3 | μM | 149.9 | 2.33 | 572.69 |
| X112 | 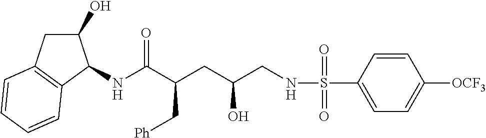 [SMILES: O=C(N[C@H]1c2ccccc2C[C@H]1O)[C@H](Cc1ccccc1)C[C@H](O)CNS(=O)(=O)c1ccc(OC(F)(F)F)cc1] | 152 | μM | 125.0 | 4.44 | 578.60 |
| X113 | 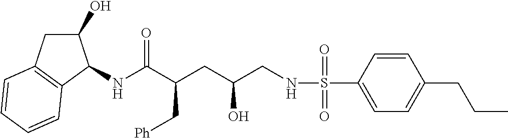 [SMILES: CCCc1ccc(S(=O)(=O)NC[C@@H](O)C[C@@H](Cc2ccccc2)C(=O)N[C@H]2c3ccccc3C[C@H]2O)cc1] | 4.20 | μM | 115.7 | 4.61 | 536.68 |
| X114 | 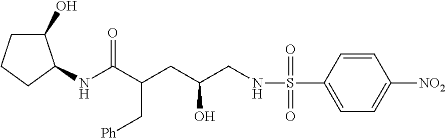 [SMILES: O=C(N[C@H]1CCC[C@H]1O)C(Cc1ccccc1)C[C@H](O)CNS(=O)(=O)c1ccc([N+](=O)[O-])cc1] | NC* | | 167.5 | 2.54 | 491.56 |
| X115 | 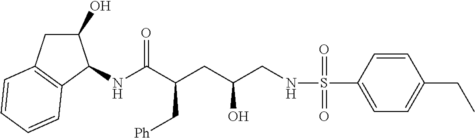 [SMILES: CCc1ccc(S(=O)(=O)NC[C@@H](O)C[C@@H](Cc2ccccc2)C(=O)N[C@H]2c3ccccc3C[C@H]2O)cc1] | 45.8 | μM | 115.7 | 4.08 | 522.66 |
| X116 | 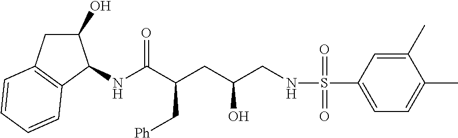 [SMILES: Cc1ccc(S(=O)(=O)NC[C@@H](O)C[C@@H](Cc2ccccc2)C(=O)N[C@H]2c3ccccc3C[C@H]2O)cc1C] | 13.3 | μM | 115.7 | 4.00 | 522.66 |
| X117 | 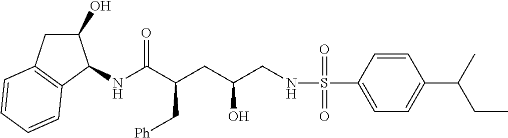 [SMILES: CCC(C)c1ccc(S(=O)(=O)NC[C@@H](O)C[C@@H](Cc2ccccc2)C(=O)N[C@H]2c3ccccc3C[C@H]2O)cc1] | 152 | μM | 115.7 | 5.01 | 550.71 |
| X118 | 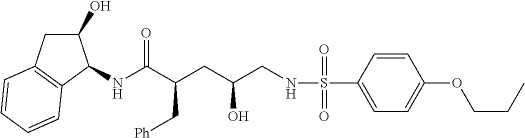 [SMILES: CCCOc1ccc(S(=O)(=O)NC[C@@H](O)C[C@@H](Cc2ccccc2)C(=O)N[C@H]2c3ccccc3C[C@H]2O)cc1] | 4.20 | μM | 125.0 | 4.39 | 552.68 |
| X119 | 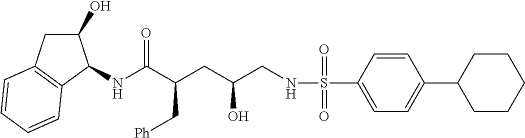 [SMILES: O=C(N[C@H]1c2ccccc2C[C@H]1O)[C@H](Cc1ccccc1)C[C@H](O)CNS(=O)(=O)c1ccc(C2CCCCC2)cc1] | NC* | | 115.7 | 5.67 | 576.75 |
| X120 | 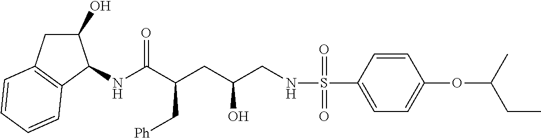 [SMILES: CCC(C)Oc1ccc(S(=O)(=O)NC[C@@H](O)C[C@@H](Cc2ccccc2)C(=O)N[C@H]2c3ccccc3C[C@H]2O)cc1] | 45.8 | μM | 125.0 | 4.70 | 566.71 |

*NC indicates that there was No Change in BRET ratio (<0.1) in response to treatment with the compound in the BRET assay.

TABLE 4

| | | | | | | Stability⁺ |
|---|---|---|---|---|---|---|
| | | | | Lipo- | | 0 = Poor |
| | | | tPSA | philicity | MW | 1 = Average |
| Name | Structure | IC50 | Å | (cLogP) | (g/mol) | 2 = Good |
| X121 | 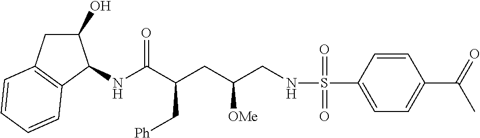 [SMILES: CO[C@H](CNS(=O)(=O)c1ccc(C(C)=O)cc1)C[C@@H](Cc1ccccc1)C(=O)N[C@H]1c2ccccc2C[C@H]1O] | NC* | 122 | 3.94 | 550.7 | 0 |
| X122 | 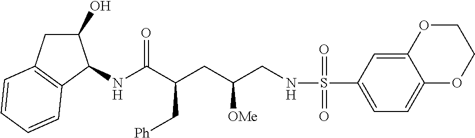 [SMILES: CO[C@H](CNS(=O)(=O)c1ccc2c(c1)OCCO2)C[C@@H](Cc1ccccc1)C(=O)N[C@H]1c2ccccc2C[C@H]1O] | NC* | 123 | 4.10 | 566.7 | 1 |
| X123 | 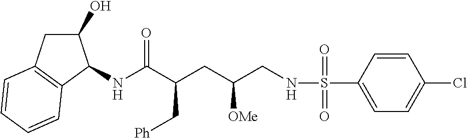 [SMILES: CO[C@H](CNS(=O)(=O)c1ccc(Cl)cc1)C[C@@H](Cc1ccccc1)C(=O)N[C@H]1c2ccccc2C[C@H]1O] | NC* | 105 | 4.87 | 543.1 | 2 |
| X124 | 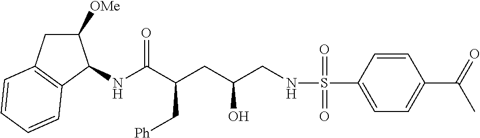 [SMILES: CO[C@@H]1Cc2ccccc2[C@@H]1NC(=O)[C@H](Cc1ccccc1)C[C@H](O)CNS(=O)(=O)c1ccc(C(C)=O)cc1] | 76.2 5 μM | 122 | 3.90 | 550.7 | 0 |
| X125 | 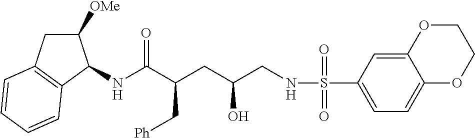 [SMILES: CO[C@@H]1Cc2ccccc2[C@@H]1NC(=O)[C@H](Cc1ccccc1)C[C@H](O)CNS(=O)(=O)c1ccc2c(c1)OCCO2] | NC* | 123 | 4.06 | 566.7 | 1 |
| X126 | 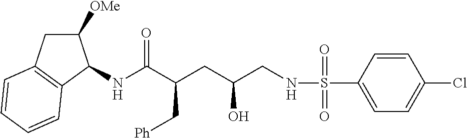 [SMILES: CO[C@@H]1Cc2ccccc2[C@@H]1NC(=O)[C@H](Cc1ccccc1)C[C@H](O)CNS(=O)(=O)c1ccc(Cl)cc1] | 13.3 4 μM | 105 | 4.83 | 543.14 | 2 |
| X127 | 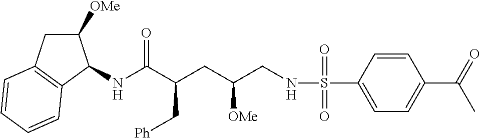 [SMILES: CO[C@H](CNS(=O)(=O)c1ccc(C(C)=O)cc1)C[C@@H](Cc1ccccc1)C(=O)N[C@H]1c2ccccc2C[C@H]1OC] | NC* | 111 | 4.70 | 564.7 | 0 |
| X128 | 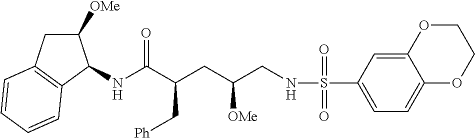 [SMILES: CO[C@H](CNS(=O)(=O)c1ccc2c(c1)OCCO2)C[C@@H](Cc1ccccc1)C(=O)N[C@H]1c2ccccc2C[C@H]1OC] | NC* | 112 | 4.86 | 580.7 | 1 |
| X129 | 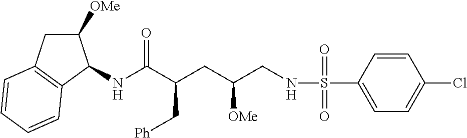 [SMILES: CO[C@H](CNS(=O)(=O)c1ccc(Cl)cc1)C[C@@H](Cc1ccccc1)C(=O)N[C@H]1c2ccccc2C[C@H]1OC] | NC* | 94 | 5.63 | 557.14 | 2 |
| X130 | 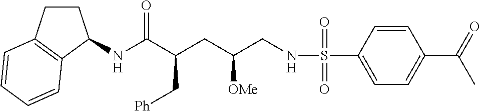 [SMILES: CO[C@H](CNS(=O)(=O)c1ccc(C(C)=O)cc1)C[C@@H](Cc1ccccc1)C(=O)N[C@@H]1CCc2ccccc21] | NC* | 102 | 4.86 | 534.7 | 0 |
| X131 | 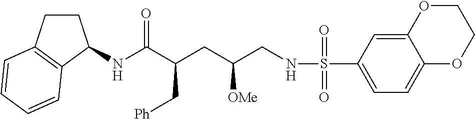 [SMILES: CO[C@H](CNS(=O)(=O)c1ccc2c(c1)OCCO2)C[C@@H](Cc1ccccc1)C(=O)N[C@@H]1CCc2ccccc21] | NC* | 103 | 5.02 | 550.7 | 1 |
| X132 | 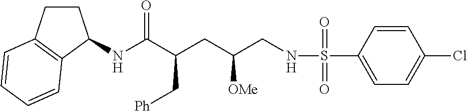 [SMILES: CO[C@H](CNS(=O)(=O)c1ccc(Cl)cc1)C[C@@H](Cc1ccccc1)C(=O)N[C@@H]1CCc2ccccc21] | NC* | 84 | 5.79 | 527.1 | 2 |
| X133 | 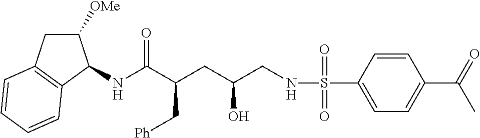 [SMILES: CO[C@H]1Cc2ccccc2[C@@H]1NC(=O)[C@H](Cc1ccccc1)C[C@H](O)CNS(=O)(=O)c1ccc(C(C)=O)cc1] | NC* | 122 | 3.90 | 550.7 | 0 |
| X134 | 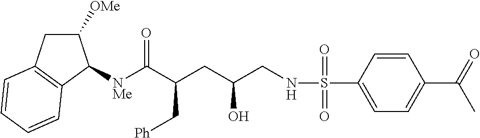 [SMILES: CO[C@H]1Cc2ccccc2[C@@H]1N(C)C(=O)[C@H](Cc1ccccc1)C[C@H](O)CNS(=O)(=O)c1ccc(C(C)=O)cc1] | NC* | 113 | 4.26 | 564.7 | 0 |
| X135 | 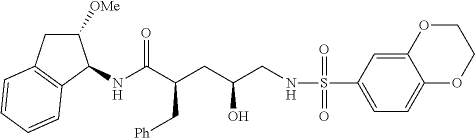 [SMILES: CO[C@H]1Cc2ccccc2[C@@H]1NC(=O)[C@H](Cc1ccccc1)C[C@H](O)CNS(=O)(=O)c1ccc2c(c1)OCCO2] | 28.1 6 μM | 123 | 4.06 | 566.7 | 1 |
| X136 | 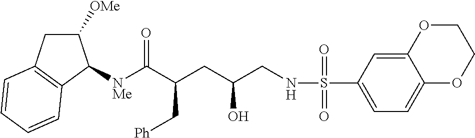 [SMILES: CO[C@H]1Cc2ccccc2[C@@H]1N(C)C(=O)[C@H](Cc1ccccc1)C[C@H](O)CNS(=O)(=O)c1ccc2c(c1)OCCO2] | NC* | 114 | 4.42 | 580.7 | 1 |
| X137 | 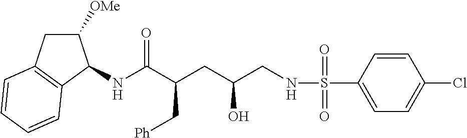 [SMILES: CO[C@H]1Cc2ccccc2[C@@H]1NC(=O)[C@H](Cc1ccccc1)C[C@H](O)CNS(=O)(=O)c1ccc(Cl)cc1] | NC* | 105 | 4.83 | 543.1 | 2 |
| X138 | 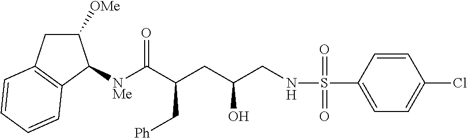 [SMILES: CO[C@H]1Cc2ccccc2[C@@H]1N(C)C(=O)[C@H](Cc1ccccc1)C[C@H](O)CNS(=O)(=O)c1ccc(Cl)cc1] | NC* | 96 | 5.19 | 557.1 | 2 |
| X139 | 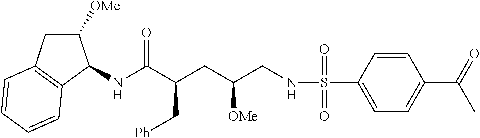 [SMILES: CO[C@H](CNS(=O)(=O)c1ccc(C(C)=O)cc1)C[C@@H](Cc1ccccc1)C(=O)N[C@H]1c2ccccc2C[C@@H]1OC] | NC* | 111 | 4.70 | 564.7 | 0 |
| X140 | 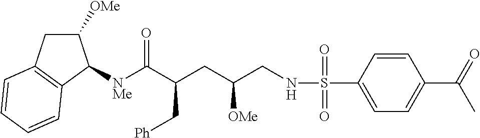 [SMILES: CO[C@H](CNS(=O)(=O)c1ccc(C(C)=O)cc1)C[C@@H](Cc1ccccc1)C(=O)N(C)[C@H]1c2ccccc2C[C@@H]1OC] | NC* | 102 | 5.06 | 578.7 | 0 |
| X141 | 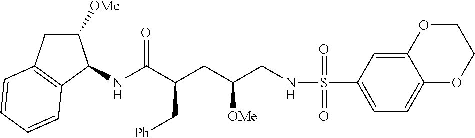 [SMILES: CO[C@H](CNS(=O)(=O)c1ccc2c(c1)OCCO2)C[C@@H](Cc1ccccc1)C(=O)N[C@H]1c2ccccc2C[C@@H]1OC] | NC* | 112 | 4.86 | 580.7 | 1 |
| X142 | 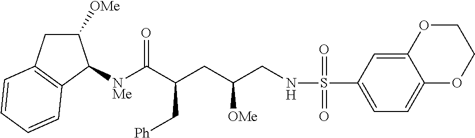 [SMILES: CO[C@H](CNS(=O)(=O)c1ccc2c(c1)OCCO2)C[C@@H](Cc1ccccc1)C(=O)N(C)[C@H]1c2ccccc2C[C@@H]1OC] | NC* | 103 | 5.22 | 594.7 | 1 |
| X143 | 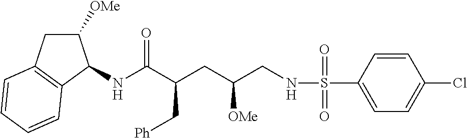 [SMILES: CO[C@H](CNS(=O)(=O)c1ccc(Cl)cc1)C[C@@H](Cc1ccccc1)C(=O)N[C@H]1c2ccccc2C[C@@H]1OC] | NC* | 94 | 5.63 | 557.1 | 2 |
| X144 | 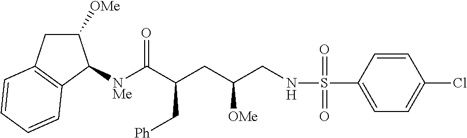 [SMILES: CO[C@H](CNS(=O)(=O)c1ccc(Cl)cc1)C[C@@H](Cc1ccccc1)C(=O)N(C)[C@H]1c2ccccc2C[C@@H]1OC] | NC* | 85 | 5.99 | 571.1 | 2 |
| AKR- 8-194 | 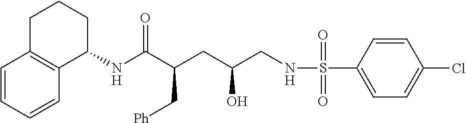 [SMILES: O=C(N[C@H]1CCCc2ccccc21)[C@H](Cc1ccccc1)C[C@H](O)CNS(=O)(=O)c1ccc(Cl)cc1] | NC* | 96 | 5.55 | 527.1 | 2 |
| AKR- 8-193 | 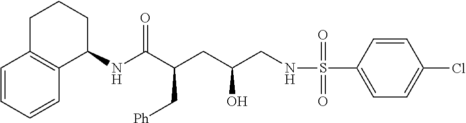 [SMILES: O=C(N[C@@H]1CCCc2ccccc21)[C@H](Cc1ccccc1)C[C@H](O)CNS(=O)(=O)c1ccc(Cl)cc1] | NC* | 96 | 5.55 | 527.1 | 2 |
| AKR- 8-186 | 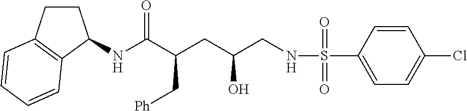 [SMILES: O=C(N[C@@H]1CCc2ccccc21)[C@H](Cc1ccccc1)C[C@H](O)CNS(=O)(=O)c1ccc(Cl)cc1] | NC* | 96 | 4.99 | 513.0 | 2 |
| AKR- 8-200 | 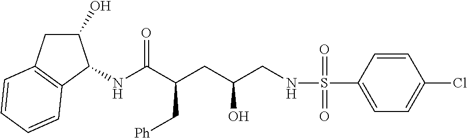 [SMILES: O=C(N[C@@H]1c2ccccc2C[C@@H]1O)[C@H](Cc1ccccc1)C[C@H](O)CNS(=O)(=O)c1ccc(Cl)cc1] | NC* | 116 | 4.07 | 529.0 | 2 |
| | 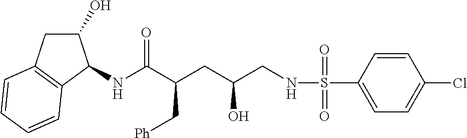 [SMILES: O=C(N[C@H]1c2ccccc2C[C@@H]1O)[C@H](Cc1ccccc1)C[C@H](O)CNS(=O)(=O)c1ccc(Cl)cc1] | | 116 | 4.07 | 529.0 | 2 |
| | 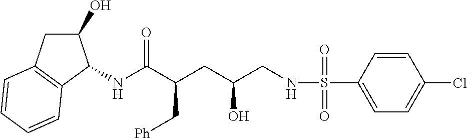 [SMILES: O=C(N[C@@H]1c2ccccc2C[C@H]1O)[C@H](Cc1ccccc1)C[C@H](O)CNS(=O)(=O)c1ccc(Cl)cc1] | | 116 | 4.07 | 529.0 | 2 |

*NC indicates that there was No Change in BRET ratio (<0.1) in response to treatment with the compound in the BRET assay.
⁺Expected stability.

### 5.2 ASSAYS

Inhibition of VIP action at the VIPR₂ receptor may be evaluated by determining whether a putative inhibitor can inhibit (e.g. reduce) a VIP-mediated increase in cAMP and/or a VIP-mediated increase in recruitment of β-arrestin, using any assay for those parameters known in the art.

In a particular, non-limiting embodiment, a Bioluminescence Resonance Energy Transfer (BRET) technique may be used to measure cAMP levels and/or β-arrestin recruitment. Unlike florescence resonance energy transfer (FRET), BRET does not require donor excitation by an external light source but uses a bioluminescent luciferase, allowing for detection of a ratiometric, high signal-to-noise signal absent photobleaching that reliably reports VIPR₂ activation.

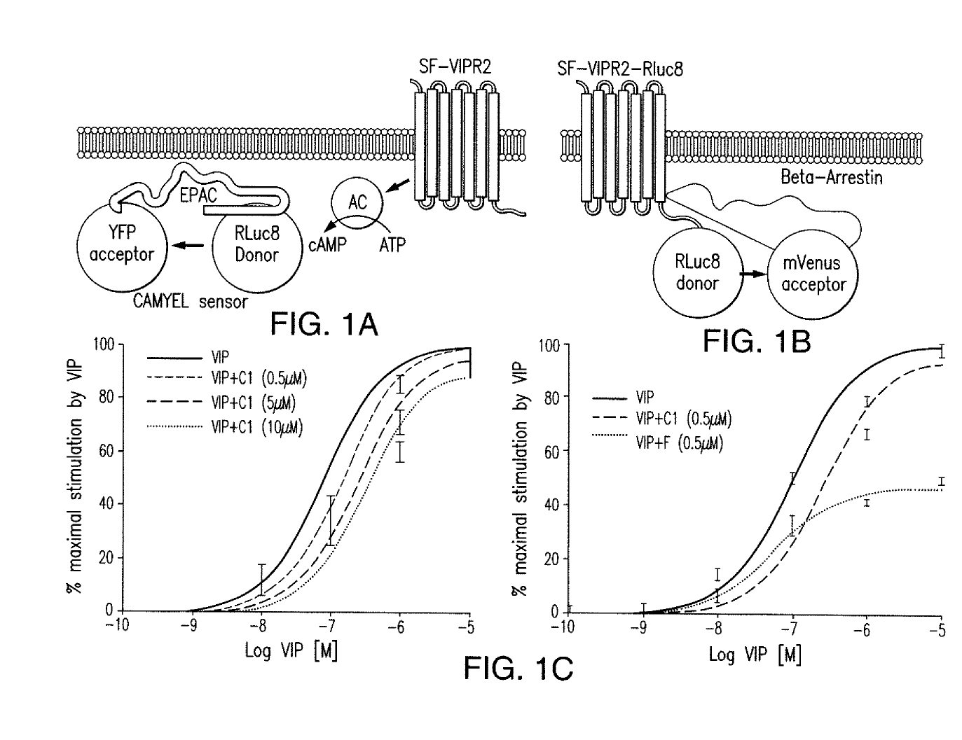

One non-limiting example of a BRET system for measuring cAMP levels is shown in FIG. 1A. In such a system, a detector cell is used which expresses both the VIPR₂ receptor and “CAMYEL,” a YFP-Epac-RLuc8 BRET sensor construct. This construct includes Epac1, a guanine nucleotide exchange factor activated by direct binding of cAMP, fused with an enhanced YFP and Renilla luciferase 8 (Rluc8) allowing BRET upon cAMP-induced conformational changes (Jiang et al., 2007, J Biol Chem. 282:10576-10584). In response to elevated cAMP levels, there is a conformational change in Epac and therefore a change in the proximity of the fused chromophores, Rluc8 and YFP, resulting in measurable energy transfer. Coexpression of this BRET construct with VIPR₂. optionally tagged at the N-terminus with a signal peptide and FLAG epitope (SF-VIPR₂), for example in HEK293 cells, allows for detection of a VIPR₂-specific response to cAMP. BRET readout may be calculated by quantifying the ratio of the light emitted by the acceptor, YFP (λ=525 nm), over the emission from the donor, RLuc8 (λ=485 nm). The proximity of the donor and acceptor, and therefore the BRET ratio, decreases in the presence of cAMP. In non-limiting specific embodiments, a stable cell line may be generated for this assay using the Flp-In T-Rex system in HEK293 cells. This system allows site-specific single copy integration of the gene of interest and control of expression levels using the Tet-repressor site making receptor expression tetracycline-inducible. For example, a CAMYEL and VIPR2 expressing line may be induced with 0.01 μg/ml tetracycline, then, 24 hours later cells may be collected and distributed into 96-well plates. After treatment with candidate inhibitor compound, for example at 5, 1 and 0.5 μM concentrations, cells may be incubated with the light emitting luciferin, coelenterazine H, for 5 min and incubated for 5 min with VIP at increasing concentrations, for example ranging from 100 pM to 10 μM. The fluorescence and luminescence may then be quantified, for example using a PHERAstar (BMG) plate reader. The degree of inhibition may be quantified by the rightward shift in the Log EC₅₀ of the VIP dose-response curve. Alternatively, analogous experiments may be performed using human cells harvested from a patient having a VIPR2 copy number variation, for example (but not by way of limitation) pluripotent stem cells prepared from such a patient and then transfected with a CAMYEL construct.

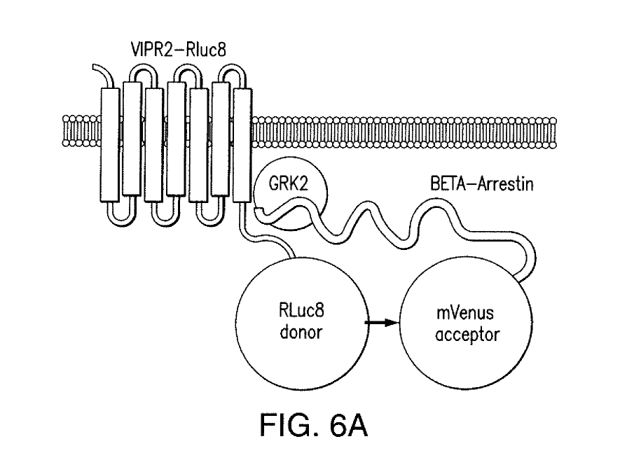

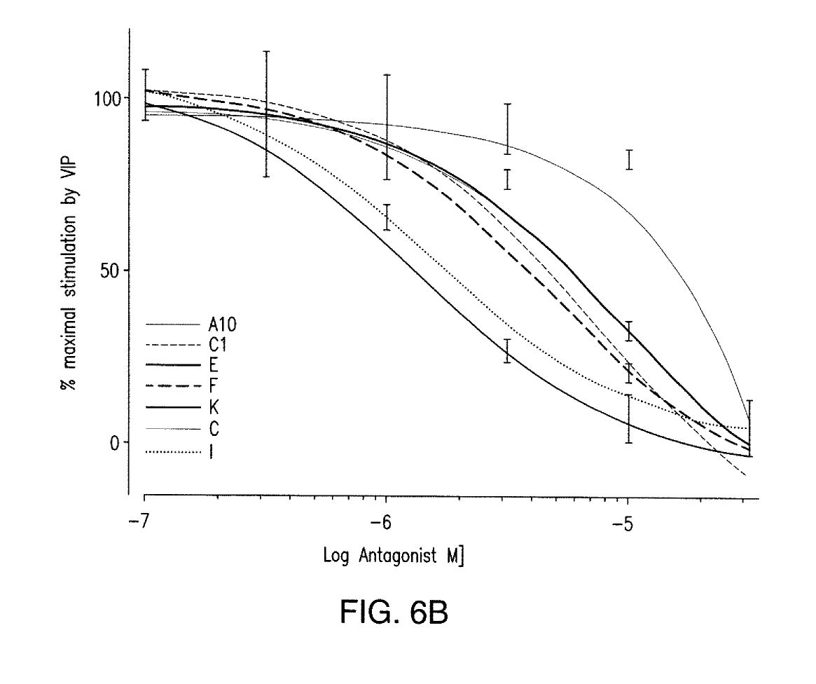

One non-limiting example of a BRET system for measuring 3-arrestin recruitment is shown in FIG. 1B and FIG. 6A. In such a system, VIP binding to VIPR₂—Rluc8 recruits (brings into proximity) mVenus-β-arrestin, resulting in measurable energy transfer. In a specific non-limiting embodiment, human mVenus-β-arrestin2 in pIRESpuro3 may be expressed together with SF-VIPR2-Rluc8. In this system, the BRET readout may be calculated by quantifying the ratio of the light emitted by the acceptor mVenus (λ=510-540 nm) over the emission from the donor RLuc8 (=485 nm). In response agonist, mVenus-β-arrestin is recruited to VIPR2-Rluc8 leading to a detectable increase in the BRET ratio. In non-limiting specific embodiments, a stable cell line may be generated for this assay using the Flp-In T-Rex system in HEK293 cells. This system allows site-specific single copy integration of the gene of interest and control of expression levels using the Tet-repressor site making receptor expression tetracycline-inducible. For example, a Venus-β-arrestin2 and VIPR2-Rluc8 expressing line may be induced with 0.01 μg/ml tetracycline, then, 24 hours later cells may be collected and distributed into 96-well plates. After treatment with candidate inhibitor compound, for example at 5, 1 and 0.5 μM concentrations, cells may be incubated with the light emitting luciferin, coelenterazine H, for 5 min and incubated for 5 min with VIP at increasing concentrations, for example ranging from 100 pM to 10 μM. The fluorescence and luminescence may then be quantified, for example using a PHERAstar (BMG) plate reader. The degree of inhibition is quantified by the rightward shift in the Log EC₅₀ of the VIP dose-response curve. Alternatively, analogous experiments may be performed using human cells harvested from a patient having a VIPR2 copy number variation, for example (but not by way of limitation) pluripotent stem cells prepared from such a patient and then transfected with a Venus-β-arrestin2 construct and an Rluc8 construct designed to express a Rluc8 which associates with intracellular VIPR₂.

In certain non-limiting embodiments, the cAMP and 3-arrestin assays described above can be conducted using cells that express a recombinant VIPR2 protein, but which do not express endogenous VIPR2. In certain embodiments, the term “endogenous VIPR2” refers to VIPR2 protein expressed by the cell that is not a recombinant VIPR2. For example, in certain embodiments, recombinant VIPR2 is the only form of VIPR2 protein expressed by the cells of the VIPR2 cellular assay.

In certain embodiments, the cells of the VIPR2 cellular assay are CHO cells, such as, for example, CHO-Flp-IN CHO cell.

In certain embodiments, a candidate compound can be identified as a VIPR2 antagonist through use of the VIPR2 cellular assay, wherein increasing concentrations of the candidate compound inhibits VIPR2 activity in the presence of a constant concentration of VIPR2 agonist.

In certain embodiments, the method of identifying a VIPR2 antagonist comprises (a) contacting a VIPR2 agonist to a cell expressing a recombinant VIPR2 protein, wherein the cell does not express endogenous VIPR2 protein, and detecting the level of cAMP in the cell; (b) contacting a candidate compound to the cell and detecting the level of cAMP in the cell; (c) comparing the level of cAMP in (a) and (b); and (d) selecting the candidate compound as a VIPR2 antagonist when the level of cAMP in (b) is less than the level of cAMP in (a).

In certain embodiments, a candidate compound can be identified as a VIPR2 agonist through use of the VIPR2 cellular assay, wherein contacting the cells of the VIPR2 cellular assay with increasing concentrations of the candidate compound increases VIPR2 activity compared to cells of the VIPR2 cellular assay not contacted with the candidate compound, or contacted with a constant concentration of a VIPR2 agonist or antagonist.

In certain embodiments, the method for identifying a VIPR2 agonist comprises (a) contacting a candidate compound to a first cell expressing a recombinant VIPR2 protein, wherein the first cell does not express endogenous VIPR2 protein; (b) detecting the level of cAMP in the first cell; (c) comparing the level of cAMP in the first cell to the level of cAMP in a second cell expressing a recombinant VIPR2 protein not contacted with the candidate compound, wherein the second cell does not express endogenous VIPR2 protein; and (d) selecting the candidate compound as a VIPR2 agonist when the cAMP level in the first cell is greater than the level of cAMP in the second cell.

In certain embodiments, cAMP level is measured using a Bioluminescence Resonance Energy Transfer (BRET) sensor, wherein binding of cAMP to the BRET sensor causes a detectable change in Bioluminescence Resonance Energy Transfer (BRET).

In certain embodiments, the BRET sensor comprises a YFP-Epac-RLuc8(CAMYEL) BRET sensor.

In certain embodiments, the method of identifying a VIPR2 antagonist comprises (a) contacting a VIPR2 agonist to a cell expressing a recombinant VIPR2 protein, wherein the cell does not express endogenous VIPR2 protein, and detecting the level of β-arrestin recruited to the recombinant VIPR2 protein in the cell; (b) contacting a candidate compound to the cell and detecting the level of β-arrestin recruited to the recombinant VIPR2 protein in the cell; (c) comparing the level of f-arrestin recruited to the recombinant VIPR2 protein in (a) and (b); and (d) selecting the candidate compound as a VIPR2 antagonist when the level of β-arrestin recruited to the recombinant VIPR2 protein in (b) is less than the level in (a).

In certain embodiments, the method of identifying a VIPR2 agonist comprises (a) contacting a candidate compound to a first cell expressing a recombinant VIPR2 protein, wherein the first cell does not express endogenous VIPR2 protein; (b) detecting the level of β-arrestin recruited to the recombinant VIPR2 protein in the first cell; (c) comparing the level of β-arrestin recruited to the recombinant VIPR2 protein in the first cell to the level of 3-arrestin recruited to a recombinant VIPR2 protein in a second cell expressing a recombinant VIPR2 protein not contacted with the candidate compound, wherein the second cell does not express endogenous VIPR2 protein; (d) selecting the candidate compound as a VIPR2 agonist when the level of β-arrestin recruited to the recombinant VIPR2 protein in the first cell is greater than the level in the second cell.

In certain embodiments, the cells express a Bioluminescence Resonance Energy Transfer (BRET) sensor, wherein recruitment of 3-arrestin to the recombinant VIPR2 protein causes a detectable change in Bioluminescence Resonance Energy Transfer (BRET).

In certain embodiments, the BRET sensor comprises an mVenus-3-arrestin2 construct and a VIPR2-RLuc8 construct.

In a further non-limiting embodiment of the invention, the ability of a compound to inhibit VIPR₂ and thereby result in a VIP-induced increase in cAMP may be measured using a Homogeneous Time-Resolved Fluorescence (“HTRF® assay; Cisbio Bioassays) as used in Chu et al., 2010, Molecular Pharmacol. 7795-101. In a method as used in Chu et al., 2010, Molecular Pharmacol. 7795-101, HEK293 cells may be transfected with nucleic acid encoding VIPR₂, for example human VIPR₂ (“hVIPR₂”), for example comprised in a vector such as pCDNA3.1 vector. Successful transformnnants may then be selected, for example using 800 μg/ml G418. Clonal stable cell lines may then be generated by limited dilution to single cells and then may be clonally expanded and tested for VIP-dependent cAMP response. For the cAMP assay, about 3000-15,000 cells (in about 4-25 μl) may be placed in a well of an assay plate. The next day, inhibitor or test inhibitor and VIP may be added in a volume about 1-2 percent of the initial volume. Assay plates may then be returned to a cell incubator for 30 min before addition of a one-half volume of cAMP conjugate and, relative to the amount of cAMP conjugate, an equal volume of anti-cAMP conjugate (Cisbio). After at least 1 h of room-temperature incubation, HTRF signal may be read, for example using Viewlux or EnVision (PerkinElmer Life and Analytical Sciences, Waltham, Mass.). The ratio of absorbance at 665 nm and 620 nm times 10,000 may be calculated and plotted.

In a further non-limiting embodiment of the invention, the ability of a compound to inhibit VIPR₂ GPCR activity may be tested, for example, using the PathHunter® eXpress 3-Arrestin GPCR system (Discoverx Corporation, Fremont, Calif., US), as used by Chu et al., 2010, Molecular Pharmacol. 7795-101. In this assay, J-Arrestin is fused to the “Enzyme Acceptor” (“EA”), an N-terminal deletion mutant of β-gal, and the GPCR of interest is fused to a smaller (42 amino acids), weakly complementing portion of the β-gal enzyme (termed “ProLink™”). In cells that stably express these fusion proteins, the interaction of β-Arrestin and the GPCR following ligand stimulation forces the complementation of the two β-gal fragments resulting in the formation of a functional enzyme that converts substrate to detectable signal. In the absence of an interaction between the GPCR and β-Arrestin, the enzyme activity is low due to the low affinity of the two enzyme fragments. For example, a nucleic acid encoding VIPR₂, for example hVIPR₂, may be cloned into the ProLink vector (DiscoveRx) for GPCR-ProLink fusion protein production. Parental HEK293 cells that stably express β-arrestin2-β-gal-EA fusion protein (DiscoveRx) may be detached and transiently transfected with the VIPR₂-containing vector using Fugene6 transfection reagent in suspension mode. Transfected cells in assay medium may be plated into test plates, for example at 15,000 cells/25 μl/well. After overnight incubation, 500 n1 of an inhibitor or test inhibitor may be introduced into the test plate followed by 2 h incubation at 37° C., 5% CO₂. Flash detection reagents may be added at 12.5 μl/well. After 5 min to 1 h of room-temperature incubation, the cell plates may be read on CLIPR (PerkinElmer Life and Analytical Sciences) or Acquest (Molecular Devices, Sunnyvale, Calif.) for luminescence signal.

In a further non-limiting embodiment, activity of a putative VIPR₂ inhibitor may be evaluated by measuring GABAergic signalling. Activation of VIPR₂ has been observed to increase evoked NMDA currents via the cyclic AMP/PKA pathway and therefore may also modulate GABAergic signaling (Yang et al., 2010, J Mol Neurosci. 42: 319-326). Accordingly, electrophysiological assays as described in Mukai et al., 2008, Nat Neurosci. 11:1302-1310 may be used to evaluate putative (test) inhibitor activity. To determine the intrinsic excitability of neurons (and to confirm neuronal maturation), whole-cell recordings may be generated at different time points. Passive membrane properties may be characterized by measuring resting membrane potential, input resistance and cell capacitance. In current-clamp recordings may be used to determine action potential threshold and firing patterns evoked by depolarizing current injections. Voltage-clamp recordings may be used to quantify the functional expression of voltage-gated sodium and potassium currents. Initial investigations of synaptic properties may optionally utilize a low-chloride, cesium-based internal solution that may allow recordation of isolated glutamatergic and GABAergic events from each neuron (by holding the cell at the chloride or cation reversal potential, respectively). Spontaneous network activity may be assayed by recording synaptic activity in the absence of tetrodotoxin in the cultures. Optionally, tetrodotoxin may then be added to the culture to block neuronal firing and allow recordation of miniature synaptic currents. The frequency of these events may be indicative of the number of functional synapses formed and their amplitude and kinetics would indicate (primarily) the properties of the postsynaptic AMPA/GABAA receptors. Further, the NMDA receptor component of excitatory synaptic events may be evaluated by recording mEPSCs in an external solution containing the co-agonist glycine and a low concentration of magnesium. Reversal of these physiological properties by treatment with putative inhibitor compound may then be assayed.

In a further non-limiting embodiment, the ability of a compound, for example an inhibitor or test inhibitor (meaning a putative inhibitor), to cross the blood brain barrier and therefore be “CNS accessible” may be evaluated using an assay known in the art such as, but not limited to, Parallel Membrane Permeability Assay (“PAMPA”)-BBB, the MDRf-MDCK11 assay, bovine brain endothelial cells, and in silico methods (see Di et al., 2009, J. Pharm. Sci. 98(6):1980-1991, Nicolazzo et al., 2006, J. Pharm. Pharmacol. 58(3):281-293, Muehlbacher et al., 2011, J. Comp. Aided Mol. Des. 25(12):1095-1106, Reichel et al., 2003, Method. Mol. Med. 89(IV):307-324) and commercial assays are available (for example the Rat Brain Endothelial Cell Monolayer Assay marketed by Solvo Biotechnology). See also Van de Waterbeemd et al., 1998, J Drug Target. 6:151-165 and Lipinski et al., 2001, Adv. Drug Deliv. Rev. 46:3-26). 5.3 ANIMAL MODEL SYSTEMS

The present invention further provides for an animal model system that may be used to evaluate putative inhibitor compounds disclosed herein for CNS VIPR₂ inhibitory activity. Said model system may be used to test the effect(s) of a compound of the invention on animal behavior as well as the pharmakokinetics of the compound, its ability to access the CNS, etc.

In particular embodiments, said animal model system introduces a region of the model animal genome that contains a VIPR₂ CNV. For example, where the model animal is a mouse, a region of murine chromosome containing a VIPR₂ CNV is introduced into a mouse.

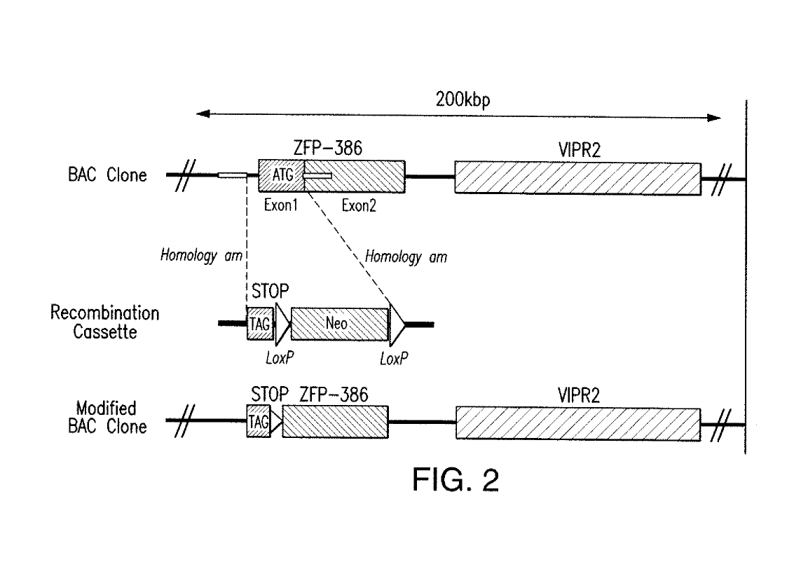

As a specific non-limiting embodiment, a murine model system may be generated using RP24-257A22 BAC, identified from the NCBI clone registry, obtained from BACPAC CHORI. The 100-kb upstream in the mouse VIPR2 encoding region contains an additional gene non-syntenic to any neighboring genes in the upstream region of the 7q36.3 human duplication. This gene encodes for a zinc-finger protein of unknown function, ZFP-386. In order to avoid any influence by ZFP-386 in test results, expression of ZFP-386 may be reduced or prevented, for example via the removal of the translation initiation region (for example, using recombineering techniques which allow homologous recombination mediated by λ phage RED system to introduce changes for large genomic vectors, such as BACs, where traditional cloning methods would not be feasible; Sharan et al., 2009, Nat Protoc. 4:206-223). In one specific non-limiting embodiment, RP24-257A22 may be introduced into the SW106 cell line, a derivative of the E. Coli EL250 line. A LoxP-Neo-LoxP (LNL) cassette may then be inserted which carries a region of the ZFP-386 with the start region deletion (FIG. 2). Following addition of arabinose in the medium the Floxed Neo may be removed. Pronuclear injection or other standard techniques may be used to derive mouse lines expressing this modified BAC region against the C57B16 background.

Due to the human uniqueness of psychiatric disorders, animal models for neuropsychiatric illness do not fully recapitulate the disease but typically show behavioral and physiological phenotypes that mimic specific disease symptoms (Nestler and Hyman, 2010, Nat Neurosci. 13:1161-1169); nevertheless, behavioral deficits in mice lines carrying SCZ-associated mutations have been characterized (Kvajo et al., 2008, Proc Natl Acad Sci USA. 105:7076-7081; Stark et al., 2008, Nat Genet. 40:751-760; Mukai et al., 2008, Nat Neurosci. 11:1302-1310; Stark et al., 2009, Int J Neuropsychopharmacol. 12(7):983-9). A murine model system may accordingly be used according to the invention to evaluate the effect of a putative inhibitor compound on behavior which serves as an indicator of effectiveness of the compound as treatment for SCZ. Further, the behavior of the murine model in any of the following tests may also be compared (in untreated animals) with that of wild-type to assist in the assessment of the validity of the model. In one specific non-limiting embodiment, the effect of a putative inhibitor compound on hyperactivity in response to stress and novel cues may be evaluated as an indicator of efficacy for treating SCZ. This assay may be performed by measuring total path length travelled over a 1-hr exposure period of wild-type (wt) and VIPR₂ CNV model mice to a novel open-field environment. In another specific non-limiting embodiment, the effect of a putative inhibitor compound on disrupted PPI, which is the reduction in startle response to successive cues, may be tested as an indicator of efficacy for treating SCZ (Wynn et al., 2004, Biological Psychiatry. 55:518-523). PPI occurs in mice and can be assayed reliably providing a highly specific correlate between the human phenotype and mouse models for the disease. PPI tests may be carried out together with acoustic startle responses and measured as previously described (Stark et al., 2009, Int J Neuropsychophannacol. 12t7):983-9). To evaluate the effect of a putative inhibitor on negative symptoms of SCZ, such as apathy and anhedonia, standard tests may be used, such as the forced swim test, by measuring the duration of escape-directed behaviors, with immobility indicating reduced motivation. The sucrose preference test can also be used as a measure of anhedonia in which mice show a reduced preference for sucrose versus water and will be carried out as described (Clapcote et al., 2007, Neuron. 54:387-402). Cognitive defects may be measured using tests of working memory (WM), fear learning and the five-choice serial reaction time task (5CSRTT). WM tasks may be used to measure learning deficits in the arm choice accuracy test, as previously described (Aultman et al., 2001, Psychopharmacology (Berl). 153:353-364). Fear conditioning assays may also be carried out to measure associative learning and memory (Stark et al., 2008, Nat Genet. 40:751-760). Further, the 5CSRTT test may be used, performance of which depends on PFC function and serves as a model for the human Continuous Performance Test, which has been shown to be affected in patients with SCZ (Wang et al., 2007, Schizophrenia Research. 89:293-298). This test allows separate assessment of attention, impulse control, perseverative- and reactivity-related functions in rodents (Robbins, 2002, Psychophannacology (Berl). 163:362-380). Further, evaluation of the effect of the inhibitor on the circadian rhythm may be evaluated, for example by determining the effect of the inhibitor on the circadian rhythm of spontaneous activity: over the course of 2 weeks mice may be housed in an automated actimeter under light:dark conditions of 12 hrs: 12 hrs, and ambulatory counts and average velocity may be recorded throughout this period and binned into 1-hr time intervals for analysis. Temperature of the Vipr2 transgenic mice or wt littennates, treated with putative inhibitor or not treated, may also be assayed as a second measure of circadian rhythms.

As with behavior, morphological changes associated with VIPR2 CNV may be evaluated in untreated model animals as well as in model animals treated with a putative inhibitor compound. Morphological features which may be tested include, but are not limited to, neuronal features, at the cellular and subcellular level, for example dendritic complexity, spine development and synaptogenesis.

Further, electrophysiological changes associated with VIPR2 CNV may be evaluated in untreated model animals as well as in model animals treated with a putative inhibitor compound. Electrophysiological features which may be tested include, but are not limited to, electrophysiologic activity in the hippocampus, the prefrontal cortex and the suprachiasmatic nucleus. Electrophysiologic features include but are not limited to intrinsic membrane properties (resting membrane potential, input resistance and cell capacitance), synaptic transmission and plasticity (EPSCs and EPSPs, stimulus-response curves, paired-pulse ratios, and short-term/long-term synaptic plasticity); see Drew et al., 2011, Mol Cell Neurosci. 47(4):293-305; Fénelon et al., 2011, Proc. Natl. Acad. Sci. U.S.A. 108:4447-4452; Kvajo et al., 2011, Proc. Natl. Acad. Sci. U.S.A., H. McKellar, L. J. Drew, A.-M. Lepagnol-Bestel, L. Xiao, R. J. Levy, et al., 2011, Proc. Natl. Acad. Sci. U.S.A. 108(49):E1349-58).

### 5.4 Methods of Treatment

In particular, non-limiting embodiments, the invention provides for use of a compound as set forth above, for example according to a Formula I-XXVII set forth above or as set forth in Table 1, 2, 3 or 4, bearing R groups as indicated above, and/or for salts and/or chelates thereof, or an enantiomer thereof in the treatment of a CNS disorder such as a psychiatric, behavioral or neurodevelopmental disorder. Non-limiting examples of psychiatric disorders which may be treated according to the invention include schizophrenia, bipolar disorder, borderline personality disorder, schizoid disorder, major depression and obsessive compulsive disorder. Non-limiting examples of neurodevelopmental disorders which may be treated according to the invention include an autism spectrum disorder, for example autism, Aspergers syndrome childhood disintegrative disorder, Rett syndrome, or pervasive developmental disorder not otherwise specified. Non-limiting examples of behavioral disorders which may be treated according to the invention include sleep disorders such as insomnia, narcolepsy, sleep deprivation).

In one particular, non-limiting embodiment, the invention provides for use of the compound

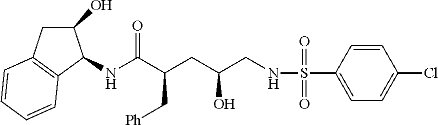 [SMILES: O=C(N[C@H]1c2ccccc2C[C@H]1O)[C@H](Cc1ccccc1)C[C@H](O)CNS(=O)(=O)c1ccc(Cl)cc1]

and/or a salt, chelate or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

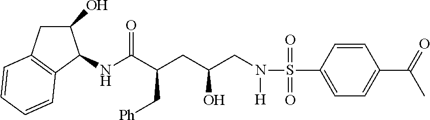 [SMILES: CC(=O)c1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

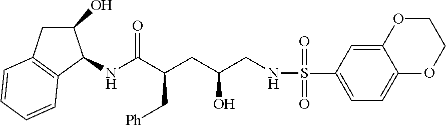 [SMILES: O=C(N[C@H]1c2ccccc2C[C@H]1O)[C@H](Cc1ccccc1)C[C@H](O)CNS(=O)(=O)c1ccc2c(c1)OCCO2]

and/or a salt, chelate or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

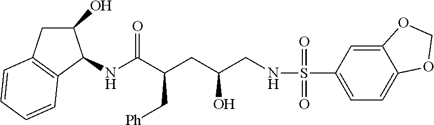 [SMILES: O=C(N[C@H]1c2ccccc2C[C@H]1O)[C@H](Cc1ccccc1)C[C@H](O)CNS(=O)(=O)c1ccc2c(c1)OCO2]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

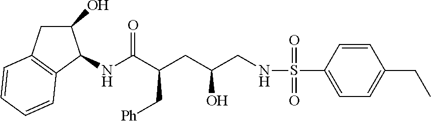 [SMILES: CCc1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

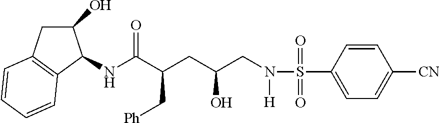 [SMILES: N#Cc1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

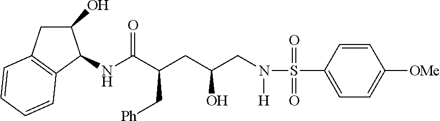 [SMILES: COc1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

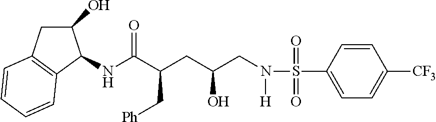 [SMILES: O=C(N[C@H]1c2ccccc2C[C@H]1O)[C@H](Cc1ccccc1)C[C@H](O)CNS(=O)(=O)c1ccc(C(F)(F)F)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

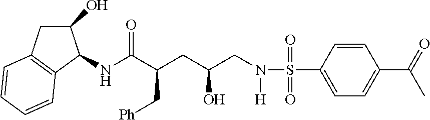 [SMILES: CC(=O)c1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

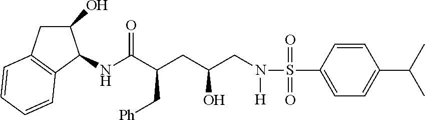 [SMILES: CC(C)c1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

In one particular, non-limiting embodiment, the invention provides for use of the compound

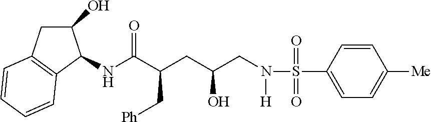 [SMILES: Cc1ccc(S(=O)(=O)NC[C@@H](O)C[C@@H](Cc2ccccc2)C(=O)N[C@H]2c3ccccc3C[C@H]2O)cc1]

and/or a salt, chelate, or enantiomer thereof, in the treatment of a CNS disorder such as a psychiatric, behavioral or neurodevelopmental disorder. In a specific, non-limiting embodiment, the disorder is schizophrenia.

Accordingly, in certain non-limiting embodiments, the present invention provides for a method of treating a subject suffering from a CNS disorder such as a psychiatric, behavioral or neurodevelopmental disorder, comprising administering, to the subject, an effective amount of a compound of the invention. In a specific non-limiting embodiment the disorder is schizophrenia. An effective amount is an amount that ameliorates the patient's symptoms, for example, thought disorder, affect, presence of hallucination or delusion, and/or would be expected to inhibit CNS VIPR₂ in the subject (for example, based on experimental data so that measurement in the subject himself/herself is not required), said inhibition being by at least about 5%, at least about 10%, at least about 20%, at least about 30% or at least about 50%. As an example, inhibition of VIPR₂ may be measured in vivo or using an in vitro assay, for example as set forth herein.

In specific non-limiting embodiments, the compound may be administered to a subject to achieve a concentration in the CNS of at least about 1 micromolar or at least about 0.5 micromolar or at least about 0.4 micromolar or at least about 0.3 micromolar or at least about 0.2 micromolar or at least about 0.1 micromolar or at least about 0.01 micromolar or at least about 0.005 micromolar.

In specific non-limiting embodiments, the compound may be administered to a subject at a dose of between about 50 and about 1500 mg/day or between about 100 and about 1200 mg/day or between about 150 and about 1100 mg/day or between about 200 and about 1000 mg/day or between about 250 and about 900 mg/day or between about 300 and about 800 mg/day or between about 400 and about 700 mg/day or between about 500 and about 600 mg/day.

In certain non-limiting embodiments, the compounds of the present application can be administered, for example, systemically (e.g. by intravenous injection, oral administration, inhalation, etc.), by intra-arterial, intramuscular, intradernnal, transdermal, subcutaneous, oral, intraperitoneal, intraventricular, or intrathecal administration, or may be administered by any other means known in the art.

In particular non-limiting embodiments, the invention provides for a method of treating a subject suffering from a behavioral disorder comprising testing the subject to determine whether the subject carries a CNV involving VIPR2, as set forth in International Patent Application No. PCT/US2012/020683, published as WO2012/094681, and if said CNV is present, treating the subject with a compound according to the invention as set forth above or recommending said treatment.

In particular, non-limiting embodiments, the application provides for methods for inhibiting VIPR2 activity in a cell by contacting a compound of the present application to the cell in an amount effective to inhibit or reduce VIPR2 activity.

In particular, non-limiting embodiments, the application provides for methods for inhibiting VIPR2 activity in a subject by administering a compound of the present application to the subject.

In certain embodiments, the compound is administered to the subject or contacted to the cell in an amount effective to inhibit the function of VIPR2 protein or reduces the level of functional VIPR2 protein.

In certain embodiments, the compound is administered to the subject or contacted to the cell in an amount effective to reduce or inhibit the ability of VIPR2 protein to activate cyclic-AMP signaling, for example, cyclic-AMP accumulation, or protein kinase A (PKA) activation.

In certain embodiments, the compound is administered to the subject or contacted to the cell in an amount effective to reduce or inhibit the ability of VIPR2 protein to bind to VIP.

In certain embodiments, the compound is administered to the subject or contacted to the cell in an amount effective to reduce or inhibit the ability of VIPR2 protein to regulate synaptic transmission in the hippocampus.

In certain embodiments, the compound is administered to the subject or contacted to the cell in an amount effective to reduce or inhibit the ability of VIPR2 protein to promote proliferation of neural progenitor cells, for example, in the dentate gyrus.

In certain embodiments, the compound is administered to the subject or contacted to the cell in an amount effective to reduce or inhibit the ability of VIPR2 protein to modulate circadian oscillations in, for example, the suprachiasmatic nucleus.

As described by the present application, the term “subject” may refer to a human or non-human subject. Examples of non-human subjects include dog, cat, rodent, cow, sheep, pig, or horse, to name a few.

### 6.1 Example 1

A series of compounds were prepared using a synthetic method analogous to that set forth in Scheme 1, above, and then tested for their ability to inhibit VIP-induced cAMP elevation and β-arrestin recruitment. The results are shown below in TABLE 5.

In a BRET-based assay, compound F above, also referred to as compound XX08 herein, had greater activity than Compound 1 (compound A above) (FIGURE IC).

TABLE 5
Structure and Activity of Compounds

| | | | % Activity | | | | | |
|---|---|---|---|---|---|---|---|---|
| | | | Arrestin | | | cAMP | | |
| | | Com- | 5 μM | | | 5 μM | | |
| | | pound | 1 μM | | | 1 μM | | |
| Entry | Structure | # | 0.5 μM | | | 0.5 μM | | |
| REF | 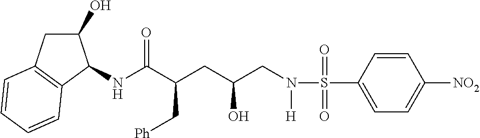 | A27 | Batch 1 88.7 74.6 37.7 | Batch 2 92.0 38.6 15.6 | Batch 3 29.3 -2.6 2.6 | Batch 1 99.1 71.3 42.6 | Batch 2 100.2 70.9 38.7 | Batch 3 97.8 86.1 74.3 |
| REF | 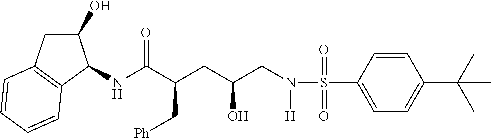 | A | 56.7 18.1 0 | | | 72.1 13 9.7 | | |
| 1 | 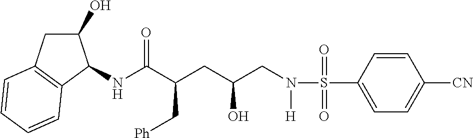 [SMILES: N#Cc1ccc(S(=O)(=O)NC[C@@H](O)C[C@@H](Cc2ccccc2)C(=O)N[C@H]2c3ccccc3C[C@H]2O)cc1] | C | 90.4 59.5 38.5 | | | 93.1 43.6 27.3 | | |
| 2 | 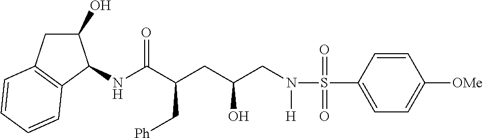 [SMILES: COc1ccc(S(=O)(=O)NC[C@@H](O)C[C@@H](Cc2ccccc2)C(=O)N[C@H]2c3ccccc3C[C@H]2O)cc1] | E | 89.4 76.7 54.1 | | | 93.8 45.9 33.9 | | |
| 3 | 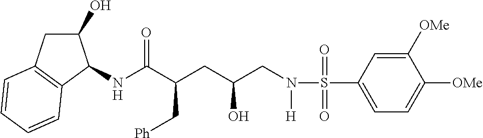 [SMILES: COc1ccc(S(=O)(=O)NC[C@@H](O)C[C@@H](Cc2ccccc2)C(=O)N[C@H]2c3ccccc3C[C@H]2O)cc1OC] | S | 32 23.2 10.5 | | | 28.2 20.4 21.1 | | |
| 4 | 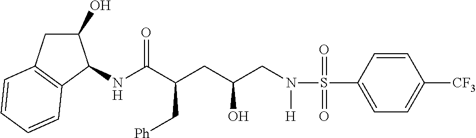 [SMILES: O=C(N[C@H]1c2ccccc2C[C@H]1O)[C@H](Cc1ccccc1)C[C@H](O)CNS(=O)(=O)c1ccc(C(F)(F)F)cc1] | F | 92.9 83 58.1 | | | 98.5 63.1 42 | | |
| 5 | 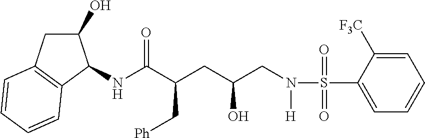 [SMILES: O=C(N[C@H]1c2ccccc2C[C@H]1O)[C@H](Cc1ccccc1)C[C@H](O)CNS(=O)(=O)c1ccccc1C(F)(F)F] | R | 21 24.8 10.7 | | | 24.7 18.8 23 | | |
| 6 | 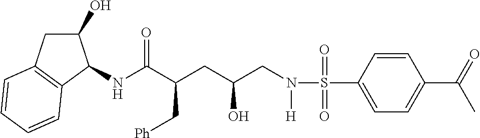 [SMILES: CC(=O)c1ccc(S(=O)(=O)NC[C@@H](O)C[C@@H](Cc2ccccc2)C(=O)N[C@H]2c3ccccc3C[C@H]2O)cc1] | K | 88.7 78 58.3 | | | 96 61.1 38.6 | | |
| 7 | 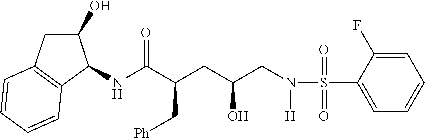 [SMILES: O=C(N[C@H]1c2ccccc2C[C@H]1O)[C@H](Cc1ccccc1)C[C@H](O)CNS(=O)(=O)c1ccccc1F] | T | 52 28.3 5.3 | | | 28.3 22.3 28.6 | | |
| 8 | 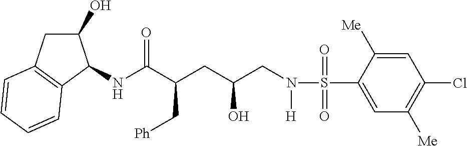 [SMILES: Cc1cc(S(=O)(=O)NC[C@@H](O)C[C@@H](Cc2ccccc2)C(=O)N[C@H]2c3ccccc3C[C@H]2O)c(C)cc1Cl] | A3 | 0.6 30.2 17.6 | | | 41.1 21.6 20.6 | | |
| 9 | 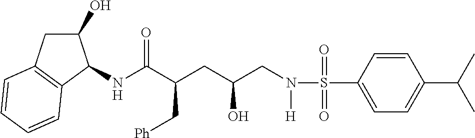 [SMILES: CC(C)c1ccc(S(=O)(=O)NC[C@@H](O)C[C@@H](Cc2ccccc2)C(=O)N[C@H]2c3ccccc3C[C@H]2O)cc1] | A10 | 87.7 63.1 31.5 | | | 98.2 44.2 36.5 | | |
| 10 | 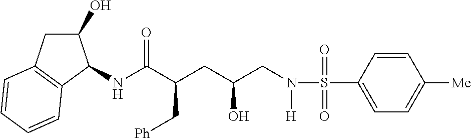 [SMILES: Cc1ccc(S(=O)(=O)NC[C@@H](O)C[C@@H](Cc2ccccc2)C(=O)N[C@H]2c3ccccc3C[C@H]2O)cc1] | I | 90 80.4 62.8 | | | 98.2 63.5 42.2 | | |
| 11 | 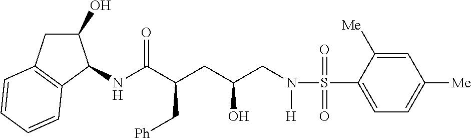 [SMILES: Cc1ccc(S(=O)(=O)NC[C@@H](O)C[C@@H](Cc2ccccc2)C(=O)N[C@H]2c3ccccc3C[C@H]2O)c(C)c1] | N | 31.8 20.3 2.8 | | | 23.3 10.9 14.1 | | |
| 12 | 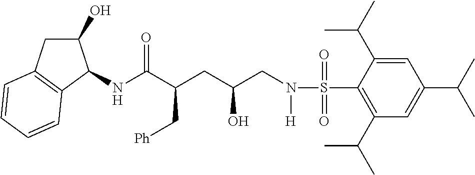 [SMILES: CC(C)c1cc(C(C)C)c(S(=O)(=O)NC[C@@H](O)C[C@@H](Cc2ccccc2)C(=O)N[C@H]2c3ccccc3C[C@H]2O)c(C(C)C)c1] | J | 8.3 30.9 29.7 | | | 90 18.6 19.5 | | |
| 13 | 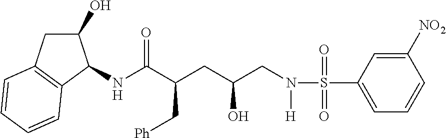 [SMILES: O=C(N[C@H]1c2ccccc2C[C@H]1O)[C@H](Cc1ccccc1)C[C@H](O)CNS(=O)(=O)c1cccc([N+](=O)[O-])c1] | L | 33.4 35.1 24.2 | | | 21.3 15.2 18.4 | | |
| 14 | 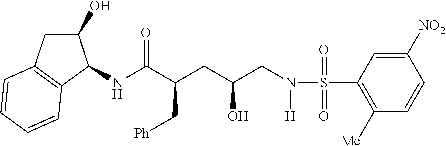 [SMILES: Cc1ccc([N+](=O)[O-])cc1S(=O)(=O)NC[C@@H](O)C[C@@H](Cc1ccccc1)C(=O)N[C@H]1c2ccccc2C[C@H]1O] | P | -0.6 -3.8 -12 | | | 18.3 13.6 10.9 | | |
| 15 | 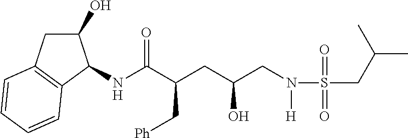 [SMILES: CC(C)CS(=O)(=O)NC[C@@H](O)C[C@@H](Cc1ccccc1)C(=O)N[C@H]1c2ccccc2C[C@H]1O] | A11 | 20 3.2 7.1 | | | 22.2 15.6 13.3 | | |
| 16 | 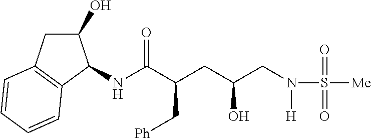 [SMILES: CS(=O)(=O)NC[C@@H](O)C[C@@H](Cc1ccccc1)C(=O)N[C@H]1c2ccccc2C[C@H]1O] | A5 | 23.4 31.6 17.3 | | | 18.5 21.9 21.4 | | |
| 17 | 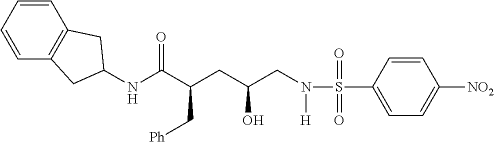 [SMILES: O=C(NC1Cc2ccccc2C1)[C@H](Cc1ccccc1)C[C@H](O)CNS(=O)(=O)c1ccc([N+](=O)[O-])cc1] | B2A | | 9.8 8.1 -2.7 | | | 16.0 7.5 5.7 | |
| 18 | 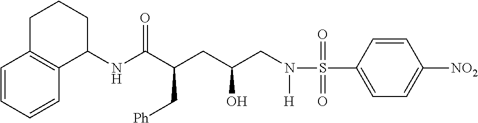 [SMILES: O=C(NC1CCCc2ccccc21)[C@H](Cc1ccccc1)C[C@H](O)CNS(=O)(=O)c1ccc([N+](=O)[O-])cc1] | B13A | | 10.2 7.4 3.3 | | | 17.5 12.4 8.8 | |
| 19 | 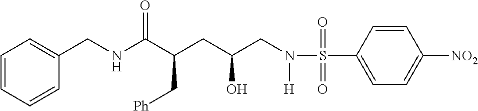 [SMILES: O=C(NCc1ccccc1)[C@H](Cc1ccccc1)C[C@H](O)CNS(=O)(=O)c1ccc([N+](=O)[O-])cc1] | B3A | | 24.2 4.4 1.8 | | | -2.9 -1.3 -1.1 | |
| 20 | 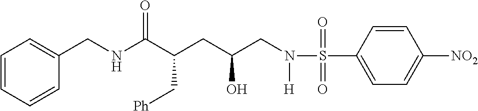 [SMILES: O=C(NCc1ccccc1)[C@@H](Cc1ccccc1)C[C@H](O)CNS(=O)(=O)c1ccc([N+](=O)[O-])cc1] | B3B | | 7.2 -4.1 -0.3 | | | 4.9 1.5 -1.0 | |
| 21 | 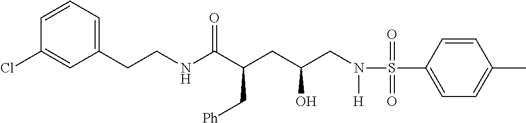 [SMILES: Cc1ccc(S(=O)(=O)NC[C@@H](O)C[C@@H](Cc2ccccc2)C(=O)NCCc2cccc(Cl)c2)cc1] | B4A | | -8.6 -7.8 -6.7 | | | 7.8 -3.4 -5.5 | |
| 22 | 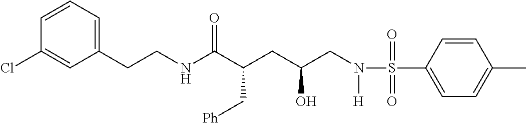 [SMILES: Cc1ccc(S(=O)(=O)NC[C@@H](O)C[C@H](Cc2ccccc2)C(=O)NCCc2cccc(Cl)c2)cc1] | B4B | | 14.7 23.4 11.1 | | | 13.0 10.7 9.3 | |
| 23 | 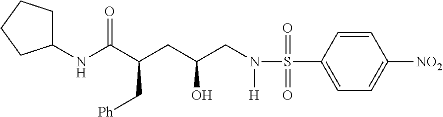 [SMILES: O=C(NC1CCCC1)[C@H](Cc1ccccc1)C[C@H](O)CNS(=O)(=O)c1ccc([N+](=O)[O-])cc1] | B1A | | 9.2 17.1 17.4 | | | 4.7 -4.9 -2.1 | |
| 24 | 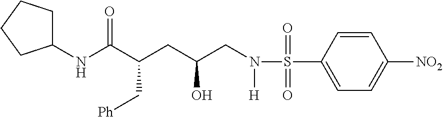 [SMILES: O=C(NC1CCCC1)[C@@H](Cc1ccccc1)C[C@H](O)CNS(=O)(=O)c1ccc([N+](=O)[O-])cc1] | B1B | | 1.3 -6.1 -5.7 | | | -0.3 -2.5 -7.5 | |
| 25 | 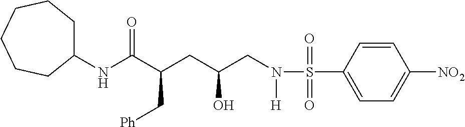 [SMILES: O=C(NC1CCCCCC1)[C@H](Cc1ccccc1)C[C@H](O)CNS(=O)(=O)c1ccc([N+](=O)[O-])cc1] | B5A | | -2.4 -5.4 -19.2 | | | 12.9 5.6 1.7 | |
| 26 | 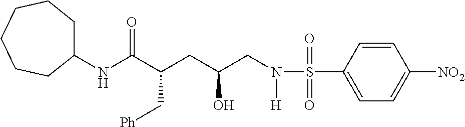 [SMILES: O=C(NC1CCCCCC1)[C@@H](Cc1ccccc1)C[C@H](O)CNS(=O)(=O)c1ccc([N+](=O)[O-])cc1] | B5B | | 6.8 3.6 -4.0 | | | 14.3 10.6 15.5 | |
| 27 | 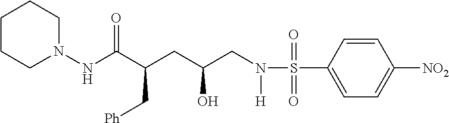 [SMILES: O=C(NN1CCCCC1)[C@H](Cc1ccccc1)C[C@H](O)CNS(=O)(=O)c1ccc([N+](=O)[O-])cc1] | B6A | 6.5 4.4 -6.4 | | | | 4.4 7.1 6.3 | |
| 28 | 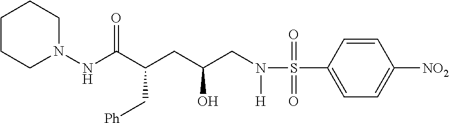 [SMILES: O=C(NN1CCCCC1)[C@@H](Cc1ccccc1)C[C@H](O)CNS(=O)(=O)c1ccc([N+](=O)[O-])cc1] | B6B | 4.7 3.3 -0.9 | | | | 6.0 12.3 9.2 | |
| 29 | 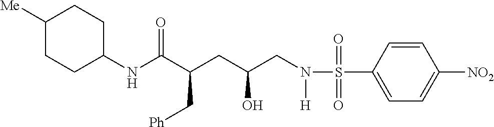 [SMILES: CC1CCC(NC(=O)[C@H](Cc2ccccc2)C[C@H](O)CNS(=O)(=O)c2ccc([N+](=O)[O-])cc2)CC1] | B8A | 0.4 -1.3 13.4 | | | | -2.8 -2.5 -5.0 | |
| 30 | 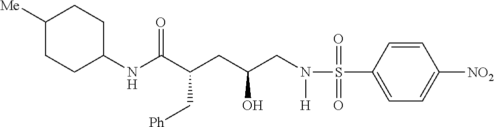 [SMILES: CC1CCC(NC(=O)[C@@H](Cc2ccccc2)C[C@H](O)CNS(=O)(=O)c2ccc([N+](=O)[O-])cc2)CC1] | B8B | | -0.8 -0.4 5.7 | | | 5.1 9.5 -0.7 | |
| 31 | 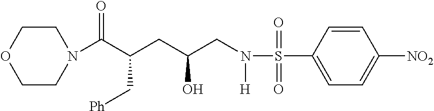 [SMILES: O=C([C@@H](Cc1ccccc1)C[C@H](O)CNS(=O)(=O)c1ccc([N+](=O)[O-])cc1)N1CCOCC1] | B9B | | 0.3 -13.0 7.0 | | | 3.0 -10.4 -5.8 | |
| 32 | 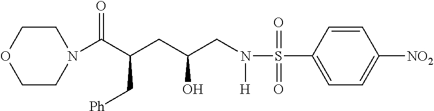 [SMILES: O=C([C@H](Cc1ccccc1)C[C@H](O)CNS(=O)(=O)c1ccc([N+](=O)[O-])cc1)N1CCOCC1] | B9A | | -1.2 8.9 -4.3 | | | 2.4 -1.6 3.8 | |
| 33 | 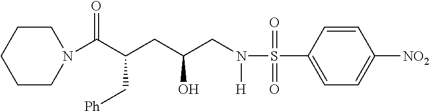 [SMILES: O=C([C@@H](Cc1ccccc1)C[C@H](O)CNS(=O)(=O)c1ccc([N+](=O)[O-])cc1)N1CCCCC1] | B18B | | 14.9 9.2 9.5 | | | 9.4 14.2 14.2 | |
| 34 | 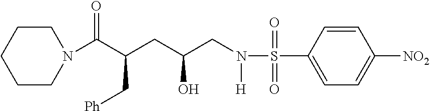 [SMILES: O=C([C@H](Cc1ccccc1)C[C@H](O)CNS(=O)(=O)c1ccc([N+](=O)[O-])cc1)N1CCCCC1] | B18A | | -2.8 -11.5 -10.2 | | | 0.5 -4.8 -4.8 | |
| 35 | 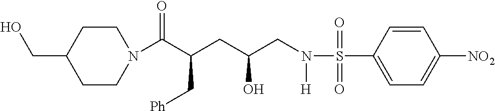 [SMILES: O=C([C@H](Cc1ccccc1)C[C@H](O)CNS(=O)(=O)c1ccc([N+](=O)[O-])cc1)N1CCC(CO)CC1] | B19 | | -4.5 -17.8 -8.4 | | | 6.2 4.4 2.1 | |
| 36 | 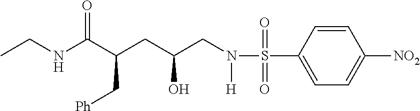 [SMILES: CCNC(=O)[C@H](Cc1ccccc1)C[C@H](O)CNS(=O)(=O)c1ccc([N+](=O)[O-])cc1] | B16A | | -12.3 0.1 15.2 | | | -6.2 -0.4 -7.1 | |
| 37 | 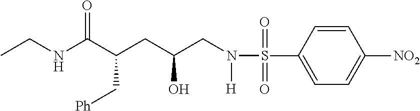 [SMILES: CCNC(=O)[C@@H](Cc1ccccc1)C[C@H](O)CNS(=O)(=O)c1ccc([N+](=O)[O-])cc1] | B16B | | -7.6 6.0 -10.0 | | | -22.2 -0.9 -13.2 | |
| 38 | 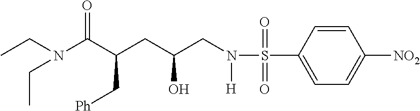 [SMILES: CCN(CC)C(=O)[C@H](Cc1ccccc1)C[C@H](O)CNS(=O)(=O)c1ccc([N+](=O)[O-])cc1] | B15A | | 5.7 7.9 -5.4 | | | -4.3 -6.1 -8.1 | |
| 39 | 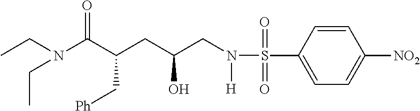 [SMILES: CCN(CC)C(=O)[C@@H](Cc1ccccc1)C[C@H](O)CNS(=O)(=O)c1ccc([N+](=O)[O-])cc1] | B15B | | -2.7 1.2 -3.6 | | | -10.5 -8.4 0.5 | |
| 40 | 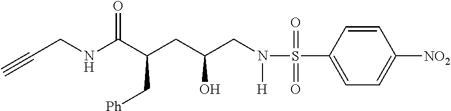 [SMILES: C#CCNC(=O)[C@H](Cc1ccccc1)C[C@H](O)CNS(=O)(=O)c1ccc([N+](=O)[O-])cc1] | B21A | | -0.2 7.2 9.4 | | | 7.3 7.8 5.4 | |
| 41 | 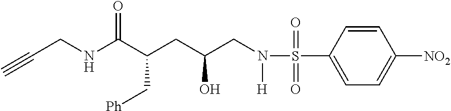 [SMILES: C#CCNC(=O)[C@@H](Cc1ccccc1)C[C@H](O)CNS(=O)(=O)c1ccc([N+](=O)[O-])cc1] | B21B | | 7.0 3.4 3.3 | | | 13.5 7.3 3.1 | |
| 42 | 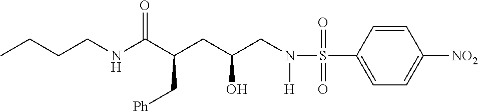 [SMILES: CCCCNC(=O)[C@H](Cc1ccccc1)C[C@H](O)CNS(=O)(=O)c1ccc([N+](=O)[O-])cc1] | B7A | | 13.8 1.9 -5.6 | | | 5.3 5.1 0.9 | |
| 43 | 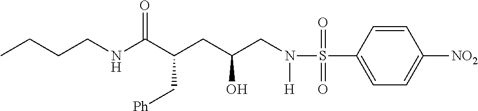 [SMILES: CCCCNC(=O)[C@@H](Cc1ccccc1)C[C@H](O)CNS(=O)(=O)c1ccc([N+](=O)[O-])cc1] | B7B | | 20.7 20.4 26.8 | | | 6.2 0.6 -1.5 | |
| 44 | 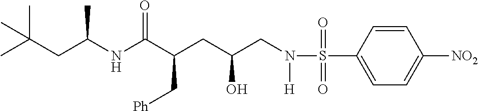 [SMILES: C[C@H](CC(C)(C)C)NC(=O)[C@H](Cc1ccccc1)C[C@H](O)CNS(=O)(=O)c1ccc([N+](=O)[O-])cc1] | B10A | | -10.3 3.0 23.3 | | | -0.1 4.4 -7.9 | |
| 45 | 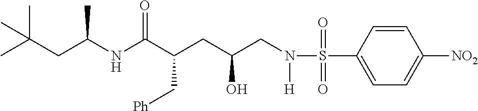 [SMILES: C[C@H](CC(C)(C)C)NC(=O)[C@@H](Cc1ccccc1)C[C@H](O)CNS(=O)(=O)c1ccc([N+](=O)[O-])cc1] | B10B | | 20.2 27.9 36.8 | | | 7.4 19.6 6.5 | |
| 46 | 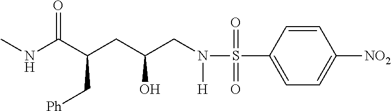 [SMILES: CNC(=O)[C@H](Cc1ccccc1)C[C@H](O)CNS(=O)(=O)c1ccc([N+](=O)[O-])cc1] | B20 | | 3.2 4.0 6.9 | | | 9.3 8.3 7.0 | |
| 47 | 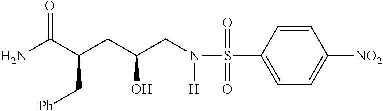 [SMILES: NC(=O)[C@H](Cc1ccccc1)C[C@H](O)CNS(=O)(=O)c1ccc([N+](=O)[O-])cc1] | B12A | | -23.0 -16.6 -6.5 | | | 2.1 3.6 6.7 | |
| 48 | 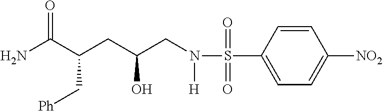 [SMILES: NC(=O)[C@@H](Cc1ccccc1)C[C@H](O)CNS(=O)(=O)c1ccc([N+](=O)[O-])cc1] | B12B | | 7.0 -9.8 3.5 | | | 9.7 12.6 14.6 | |
| 49 | 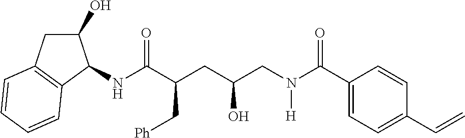 [SMILES: C=Cc1ccc(C(=O)NC[C@@H](O)C[C@@H](Cc2ccccc2)C(=O)N[C@H]2c3ccccc3C[C@H]2O)cc1] | A24A | 1.8 47.5 2.3 | | | 23.8 16.5 16.1 | | |
| 50 | 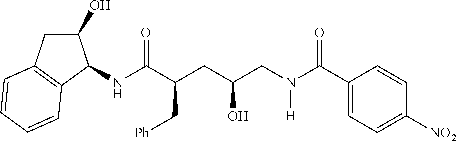 [SMILES: O=C(NC[C@@H](O)C[C@@H](Cc1ccccc1)C(=O)N[C@H]1c2ccccc2C[C@H]1O)c1ccc([N+](=O)[O-])cc1] | A25 | 16.4 18.6 -0.5 | | | 21.1 22.3 14 | | |
| 51 | 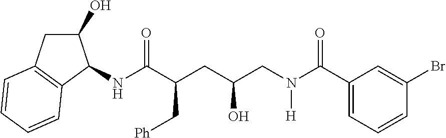 [SMILES: O=C(NC[C@@H](O)C[C@@H](Cc1ccccc1)C(=O)N[C@H]1c2ccccc2C[C@H]1O)c1cccc(Br)c1] | Q | 5.2 6.8 -3.8 | | | 20.3 18.5 17.4 | | |
| 52 | 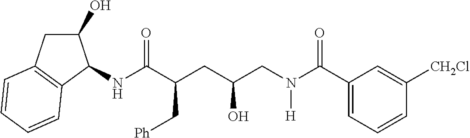 | O | -10.3 5.7 -8.1 | | | 15.1 2.3 0.5 | | |
| 53 | 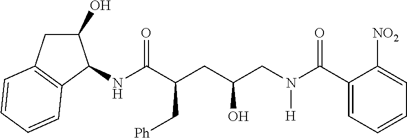 [SMILES: O=C(NC[C@@H](O)C[C@@H](Cc1ccccc1)C(=O)N[C@H]1c2ccccc2C[C@H]1O)c1ccccc1[N+](=O)[O-]] | G | 26.1 35.1 36.4 | | | 9.1 14.3 17.6 | | |
| 54 | 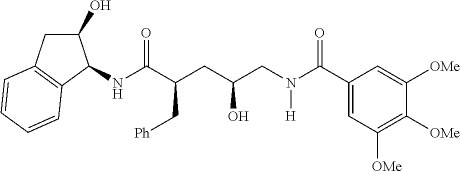 [SMILES: COc1cc(C(=O)NC[C@@H](O)C[C@@H](Cc2ccccc2)C(=O)N[C@H]2c3ccccc3C[C@H]2O)cc(OC)c1OC] | H | 18 3.2 29.6 | | | 9.3 20 15.6 | | |
| 55 | 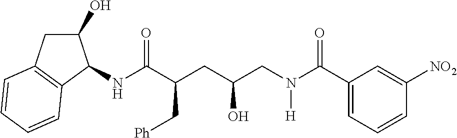 [SMILES: O=C(NC[C@@H](O)C[C@@H](Cc1ccccc1)C(=O)N[C@H]1c2ccccc2C[C@H]1O)c1cccc([N+](=O)[O-])c1] | M | -4 7.4 19.9 | | | 13.5 11 11.8 | | |
| 56 | 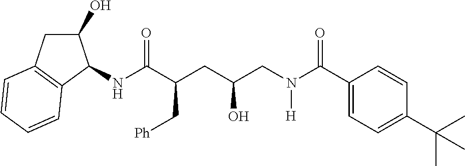 [SMILES: CC(C)(C)c1ccc(C(=O)NC[C@@H](O)C[C@@H](Cc2ccccc2)C(=O)N[C@H]2c3ccccc3C[C@H]2O)cc1] | A26 | 7.8 11.3 16.2 | | | 23.2 16 10.7 | | |
| 57 | 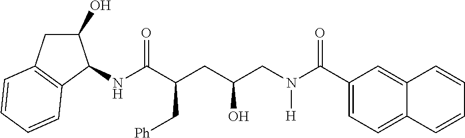 [SMILES: O=C(NC[C@@H](O)C[C@@H](Cc1ccccc1)C(=O)N[C@H]1c2ccccc2C[C@H]1O)c1ccc2ccccc2c1] | B | 9.7 11.1 -2.9 | | | 23.5 16.3 13.2 | | |
| 58 | 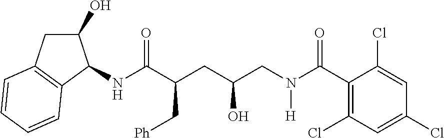 [SMILES: O=C(NC[C@@H](O)C[C@@H](Cc1ccccc1)C(=O)N[C@H]1c2ccccc2C[C@H]1O)c1c(Cl)cc(Cl)cc1Cl] | D | 31.5 25.3 23.9 | | | 12.3 14.8 17.6 | | |
| 59 | 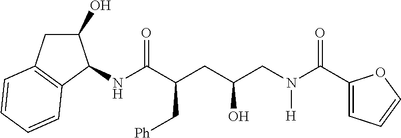 [SMILES: O=C(NC[C@@H](O)C[C@@H](Cc1ccccc1)C(=O)N[C@H]1c2ccccc2C[C@H]1O)c1ccco1] | A2 | 33.3 27.7 30.3 | | | 19.1 22.5 20.8 | | |
| 60 | 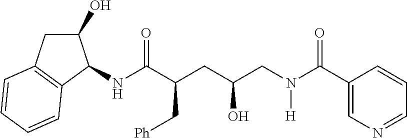 [SMILES: O=C(NC[C@@H](O)C[C@@H](Cc1ccccc1)C(=O)N[C@H]1c2ccccc2C[C@H]1O)c1cccnc1] | A16 | -6.3 -5.7 -6.8 | | | 16.4 13.5 15.2 | | |
| 61 | 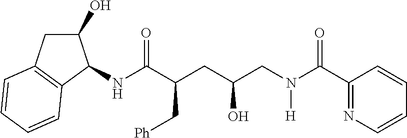 [SMILES: O=C(NC[C@@H](O)C[C@@H](Cc1ccccc1)C(=O)N[C@H]1c2ccccc2C[C@H]1O)c1ccccn1] | A17 | 5.5 32.7 6.1 | | | 20.3 17.7 14.9 | | |
| 62 | 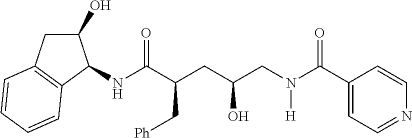 [SMILES: O=C(NC[C@@H](O)C[C@@H](Cc1ccccc1)C(=O)N[C@H]1c2ccccc2C[C@H]1O)c1ccncc1] | A18 | -8.8 10.9 -39.7 | | | 21 19.2 14.2 | | |
| 63 | 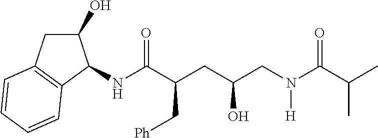 [SMILES: CC(C)C(=O)NC[C@@H](O)C[C@@H](Cc1ccccc1)C(=O)N[C@H]1c2ccccc2C[C@H]1O] | A13 | -16.3 -10.7 -11.8 | | | -0.9 2.6 0.7 | | |
| 64 | 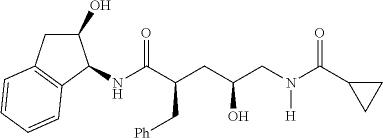 [SMILES: O=C(NC[C@@H](O)C[C@@H](Cc1ccccc1)C(=O)N[C@H]1c2ccccc2C[C@H]1O)C1CC1] | A1 | 29.1 37 -3.8 | | | 24 21.1 21 | | |
| 65 | 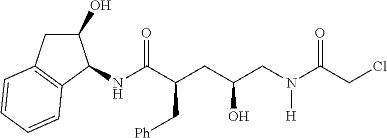 [SMILES: O=C(CCl)NC[C@@H](O)C[C@@H](Cc1ccccc1)C(=O)N[C@H]1c2ccccc2C[C@H]1O] | A7 | 17.5 23.7 9.9 | | | 15.6 19.2 18.4 | | |
| 66 | 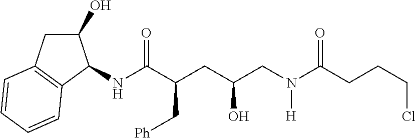 [SMILES: O=C(CCCCl)NC[C@@H](O)C[C@@H](Cc1ccccc1)C(=O)N[C@H]1c2ccccc2C[C@H]1O] | A22 | 21.1 24.3 19.2 | | | 15.9 18.2 16.4 | | |
| 67 | 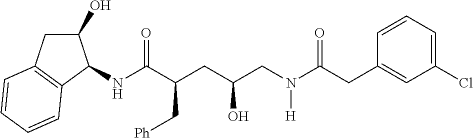 [SMILES: O=C(Cc1cccc(Cl)c1)NC[C@@H](O)C[C@@H](Cc1ccccc1)C(=O)N[C@H]1c2ccccc2C[C@H]1O] | A19 | 17.2 20.1 11.3 | | | 26.5 16 14.4 | | |
| 68 | 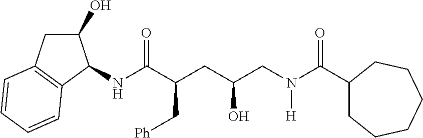 [SMILES: O=C(NC[C@@H](O)C[C@@H](Cc1ccccc1)C(=O)N[C@H]1c2ccccc2C[C@H]1O)C1CCCCCC1] | A8 | 10.5 16.4 24.4 | | | 17 17 18.4 | | |
| 69 | 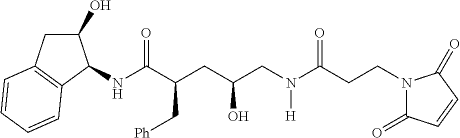 [SMILES: O=C(CCN1C(=O)C=CC1=O)NC[C@@H](O)C[C@@H](Cc1ccccc1)C(=O)N[C@H]1c2ccccc2C[C@H]1O] | A21 | -3.1 15.2 9.9 | | | 18.6 19 15.5 | | |
| 70 | 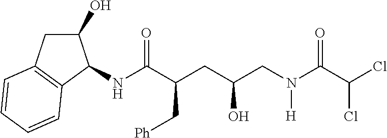 [SMILES: O=C(NC[C@@H](O)C[C@@H](Cc1ccccc1)C(=O)N[C@H]1c2ccccc2C[C@H]1O)C(Cl)Cl] | A6 | 20 17.4 9.5 | | | 19.6 18.3 18.4 | | |
| 71 | 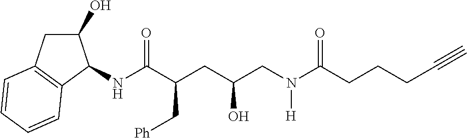 [SMILES: C#CCCCC(=O)NC[C@@H](O)C[C@@H](Cc1ccccc1)C(=O)N[C@H]1c2ccccc2C[C@H]1O] | A23 | 25.8 16.1 45.8 | | | 26.6 20.2 20.8 | | |
| 72 | 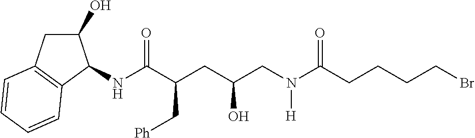 [SMILES: O=C(CCCCBr)NC[C@@H](O)C[C@@H](Cc1ccccc1)C(=O)N[C@H]1c2ccccc2C[C@H]1O] | A15 | -10.6 -28.8 1.7 | | | 9.4 12.9 11.7 | | |
| 73 | 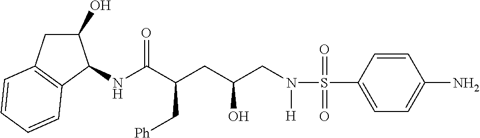 [SMILES: Nc1ccc(S(=O)(=O)NC[C@@H](O)C[C@@H](Cc2ccccc2)C(=O)N[C@H]2c3ccccc3C[C@H]2O)cc1] | X081 | | | 6.7 -5.5 -5.6 | | | 33.3 18.6 8.4 |
| 74 | 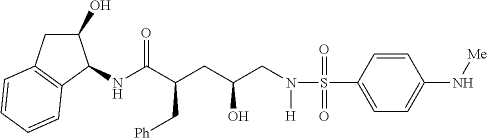 [SMILES: CNc1ccc(S(=O)(=O)NC[C@@H](O)C[C@@H](Cc2ccccc2)C(=O)N[C@H]2c3ccccc3C[C@H]2O)cc1] | X082 | | | 4.3 1.1 -0.8 | | | -7.3 2 -0.9 |
| 75 | 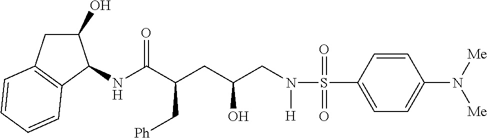 [SMILES: CN(C)c1ccc(S(=O)(=O)NC[C@@H](O)C[C@@H](Cc2ccccc2)C(=O)N[C@H]2c3ccccc3C[C@H]2O)cc1] | X083 | | | 8.7 -1.6 4.2 | | | -3.4 -1.4 9.4 |
| 76 | 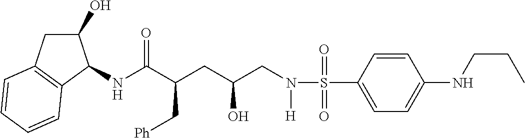 [SMILES: CCCNc1ccc(S(=O)(=O)NC[C@@H](O)C[C@@H](Cc2ccccc2)C(=O)N[C@H]2c3ccccc3C[C@H]2O)cc1] | X084 | | | 6.7 2.6 4.1 | | | 24.5 11.3 -2.9 |
| 77 | 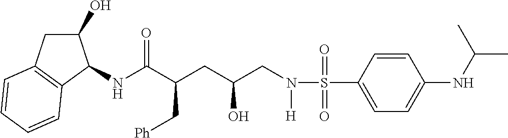 [SMILES: CC(C)Nc1ccc(S(=O)(=O)NC[C@@H](O)C[C@@H](Cc2ccccc2)C(=O)N[C@H]2c3ccccc3C[C@H]2O)cc1] | X085 | | | 11.8 -2 8.1 | | | 20.6 18.2 17.2 |
| 78 | 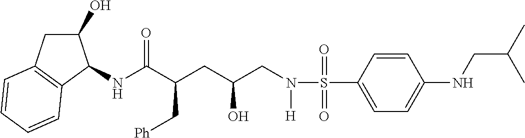 [SMILES: CC(C)CNc1ccc(S(=O)(=O)NC[C@@H](O)C[C@@H](Cc2ccccc2)C(=O)N[C@H]2c3ccccc3C[C@H]2O)cc1] | X086 | | | 11.9 5.2 10.2 | | | 27.4 16.7 21.6 |
| 79 | 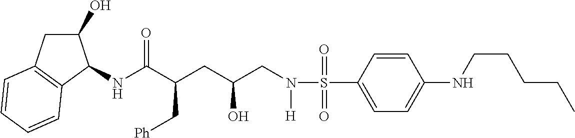 [SMILES: CCCCCNc1ccc(S(=O)(=O)NC[C@@H](O)C[C@@H](Cc2ccccc2)C(=O)N[C@H]2c3ccccc3C[C@H]2O)cc1] | X087 | | | 11.3 2.3 2.8 | | | 19.1 19.1 10.8 |
| 80 | 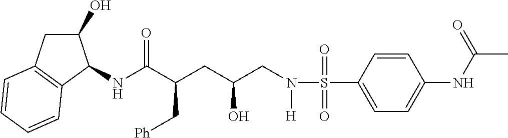 [SMILES: CC(=O)Nc1ccc(S(=O)(=O)NC[C@@H](O)C[C@@H](Cc2ccccc2)C(=O)N[C@H]2c3ccccc3C[C@H]2O)cc1] | X088 | | | 4.2 4.9 -1.2 | | | 19.1 19.1 15.2 |
| 81 | 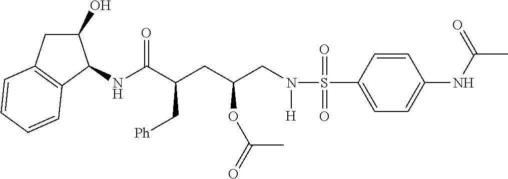 [SMILES: CC(=O)Nc1ccc(S(=O)(=O)NC[C@H](C[C@@H](Cc2ccccc2)C(=O)N[C@H]2c3ccccc3C[C@H]2O)OC(C)=O)cc1] | X089 | | | 10.5 -1.8 -9.8 | | | 3 32.3 -4.3 |
| 82 | 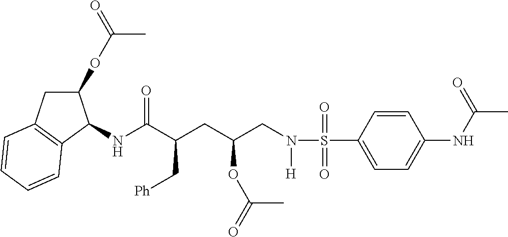 [SMILES: CC(=O)Nc1ccc(S(=O)(=O)NC[C@H](C[C@@H](Cc2ccccc2)C(=O)N[C@H]2c3ccccc3C[C@H]2OC(C)=O)OC(C)=O)cc1] | X090 | | | 9.7 0.2 0.5 | | | -5.8 -8.7 -8.7 |
| 83 | 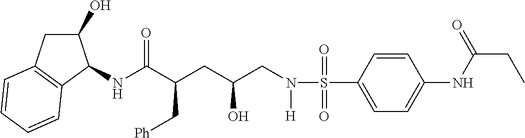 [SMILES: CCC(=O)Nc1ccc(S(=O)(=O)NC[C@@H](O)C[C@@H](Cc2ccccc2)C(=O)N[C@H]2c3ccccc3C[C@H]2O)cc1] | X091 | | | -10.4 -1.2 3.4 | | | 26.9 1.5 0.6 |
| 84 | 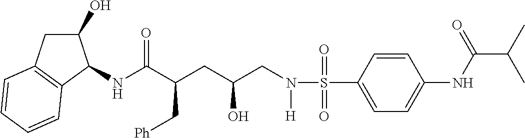 [SMILES: CC(C)C(=O)Nc1ccc(S(=O)(=O)NC[C@@H](O)C[C@@H](Cc2ccccc2)C(=O)N[C@H]2c3ccccc3C[C@H]2O)cc1] | X092 | | | -7.5 5.1 5.3 | | | 16.7 6.9 1 |
| 85 | 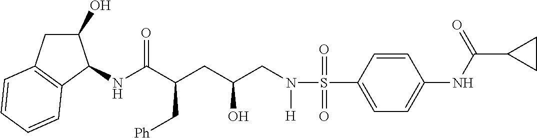 [SMILES: O=C(Nc1ccc(S(=O)(=O)NC[C@@H](O)C[C@@H](Cc2ccccc2)C(=O)N[C@H]2c3ccccc3C[C@H]2O)cc1)C1CC1] | X093 | | | -4.2 -0.3 6.4 | | | 34.3 23 -2.4 |
| 86 | 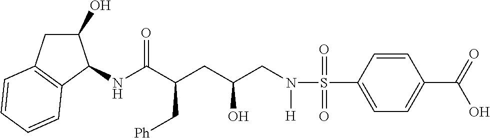 [SMILES: O=C(O)c1ccc(S(=O)(=O)NC[C@@H](O)C[C@@H](Cc2ccccc2)C(=O)N[C@H]2c3ccccc3C[C@H]2O)cc1] | X094 | | | 0.2 -1.8 6.4 | | | 26 6.4 7.4 |
| 87 | 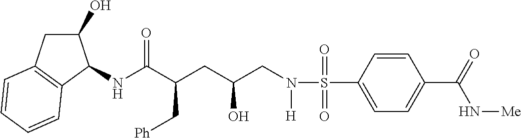 [SMILES: CNC(=O)c1ccc(S(=O)(=O)NC[C@@H](O)C[C@@H](Cc2ccccc2)C(=O)N[C@H]2c3ccccc3C[C@H]2O)cc1] | X095 | | | -1 -1.6 6.3 | | | 69.5 22.1 13.8 |
| 88 | 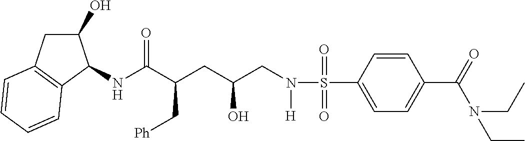 [SMILES: CCN(CC)C(=O)c1ccc(S(=O)(=O)NC[C@@H](O)C[C@@H](Cc2ccccc2)C(=O)N[C@H]2c3ccccc3C[C@H]2O)cc1] | X096 | | | -1.2 3.1 0.3 | | | 31.8 5.9 2.5 |
| 89 | 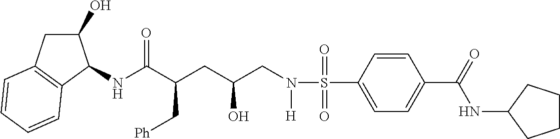 [SMILES: O=C(NC1CCCC1)c1ccc(S(=O)(=O)NC[C@@H](O)C[C@@H](Cc2ccccc2)C(=O)N[C@H]2c3ccccc3C[C@H]2O)cc1] | X097 | | | 0.1 0.5 3.9 | | | 31.8 19.6 15.2 |
| 90 | 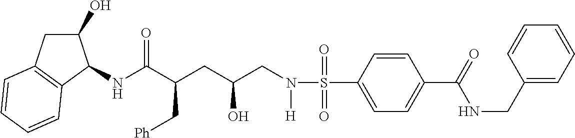 [SMILES: O=C(NCc1ccccc1)c1ccc(S(=O)(=O)NC[C@@H](O)C[C@@H](Cc2ccccc2)C(=O)N[C@H]2c3ccccc3C[C@H]2O)cc1] | X098 | | | -7.7 -2 4.8 | | | 90.5 32.3 25 |
| 91 | 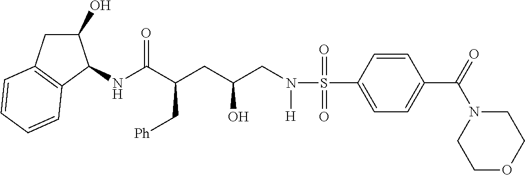 [SMILES: O=C(N[C@H]1c2ccccc2C[C@H]1O)[C@H](Cc1ccccc1)C[C@H](O)CNS(=O)(=O)c1ccc(C(=O)N2CCOCC2)cc1] | X099 | | | -5.4 1.8 4.1 | | | 25.5 0.1 -1.4 |
| 92 | 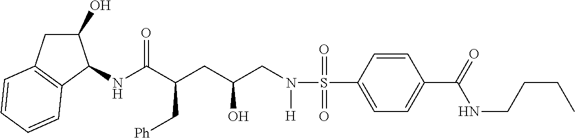 [SMILES: CCCCNC(=O)c1ccc(S(=O)(=O)NC[C@@H](O)C[C@@H](Cc2ccccc2)C(=O)N[C@H]2c3ccccc3C[C@H]2O)cc1] | X100 | | | -5.5 2.9 -0.4 | | | 20.6 5.9 -0.4 |
| 93 | 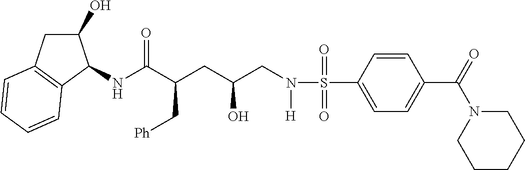 [SMILES: O=C(N[C@H]1c2ccccc2C[C@H]1O)[C@H](Cc1ccccc1)C[C@H](O)CNS(=O)(=O)c1ccc(C(=O)N2CCCCC2)cc1] | X101 | | | -12.3 -17.3 0 | | | -1.4 -7.3 -5.3 |
| 94 | 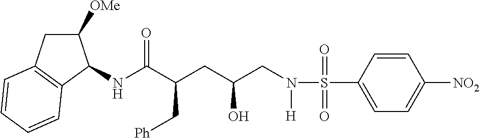 [SMILES: CO[C@@H]1Cc2ccccc2[C@@H]1NC(=O)[C@H](Cc1ccccc1)C[C@H](O)CNS(=O)(=O)c1ccc([N+](=O)[O-])cc1] | X102 | | | -10 -21.6 -7.1 | | | 5.9 5.9 15.7 |
| 95 | 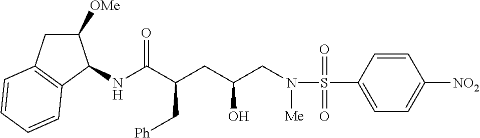 [SMILES: CO[C@@H]1Cc2ccccc2[C@@H]1NC(=O)[C@H](Cc1ccccc1)C[C@H](O)CN(C)S(=O)(=O)c1ccc([N+](=O)[O-])cc1] | X103 | | | -12.2 -11.4 4.9 | | | 26 11.3 21.1 |
| 96 | 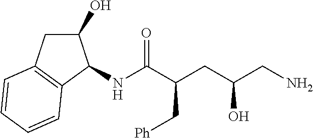 [SMILES: NC[C@@H](O)C[C@@H](Cc1ccccc1)C(=O)N[C@H]1c2ccccc2C[C@H]1O] | A28 | 19.7 1.4 3.7 | | | 15.2 15.9 11.8 | | |
| 97 | 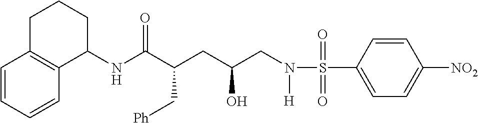 [SMILES: O=C(NC1CCCc2ccccc21)[C@@H](Cc1ccccc1)C[C@H](O)CNS(=O)(=O)c1ccc([N+](=O)[O-])cc1] | B13B | | 0.6 5.9 4.8 | | | 17.4 6.6 5.1 | |
| 98 | 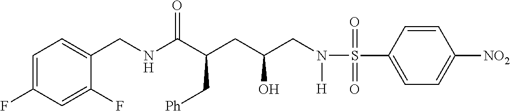 [SMILES: O=C(NCc1ccc(F)cc1F)[C@H](Cc1ccccc1)C[C@H](O)CNS(=O)(=O)c1ccc([N+](=O)[O-])cc1] | B14A | | 30.8 -13.3 -19.5 | | | 23.6 7.2 2.4 | |
| 99 | 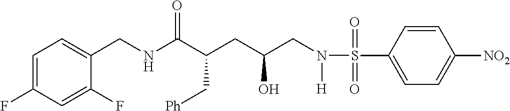 [SMILES: O=C(NCc1ccc(F)cc1F)[C@@H](Cc1ccccc1)C[C@H](O)CNS(=O)(=O)c1ccc([N+](=O)[O-])cc1] | B14B | | 24.0 20.0 23.9 | | | 18.2 4.7 -5.4 | |

### 6.2 Example 2

A series of compounds were prepared using a synthetic method analogous to that set forth in Scheme 1, above, and then tested for their ability to inhibit VIP-induced cAMP elevation and β-arrestin recruitment, as described herein (Discoverx Corporation, Fremont, Calif., US). Compound activities were also tested in a BRET-based assay, as described herein. The results are shown below in Tables 1-4, above.

### 6.3 EXAMPLE 3

Assay for the screening of VIPR2 antagonists, agonists, allosteric compounds and investigation of functionally selective compounds

An assay for medium throughput screening to enable the investigation of antagonists and agonists at VIPR2 (Vasoactive intestinal peptide receptor 2), also known as VPAC2, and other Vasopressin receptor family members was developed. The VIPR2 receptor has been implicated in the pathology of diseases including schizophrenia and carcinomas [1,2]. The discovery of novel agonists and antagonists will provide useful therapies for a wide range of diseases. VIPR2 agonists have been suggested as a possible treatment in diabetes and disorders of the immune system [3,4]. A novel BRET-based assay able to detect changes in both cAMP levels and β-arrestin recruitment in the presence of vasopressin receptors was developed. Bioluminescence Resonance Energy Transfer (BRET) is a highly robust technique. BRET does not require donor excitation by an external light source but uses a bioluminescent luciferase, Renilla luciferase 8 (Rluc8), allowing for detection of a ratiometric, high signal-to-noise signal, absent of photobleaching that reliably reports VIPR2 activation. This can be used on a time scale of seconds allowing for determination of rapid rises in the levels of second messengers such as cAMP or recruitment of β-arrestin.

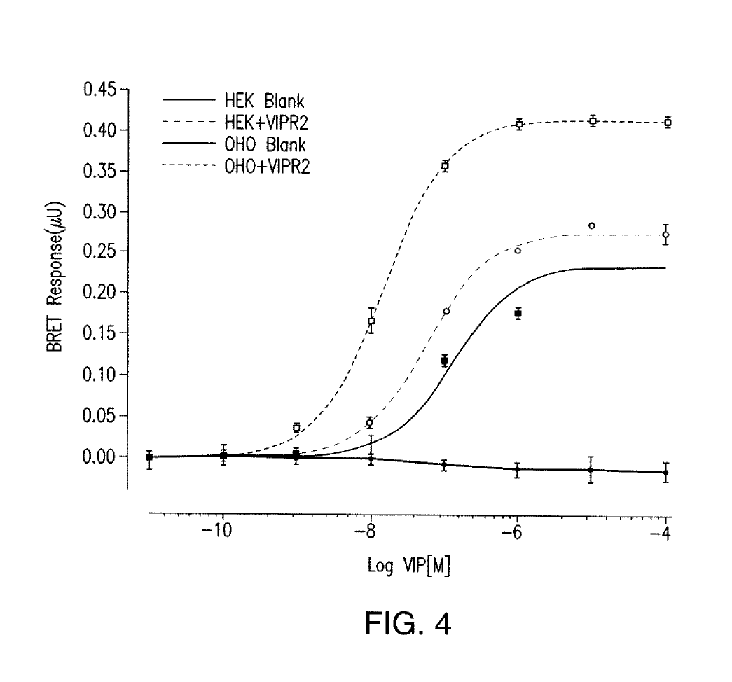

Previous published studies have demonstrated the use of an HEK293 assay for screening of VIPR2 antagonists [5]. It has been demonstrated herein that VIPR2 receptors are present endogenously in HEK293 (FIG. 4) and this is supported in the literature [6]. Other cell lines were investigated and it was discovered that the CHO cell line is absent of any receptors to vasopressin (FIG. 4).

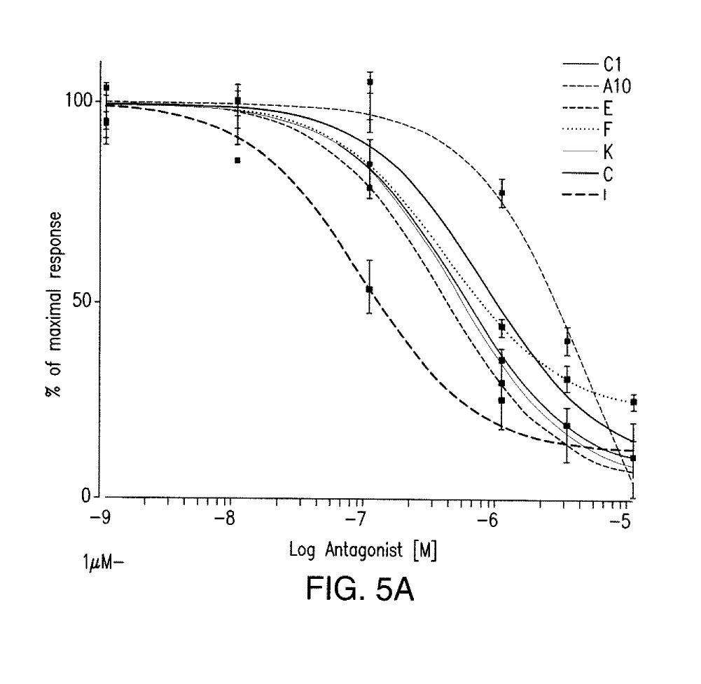

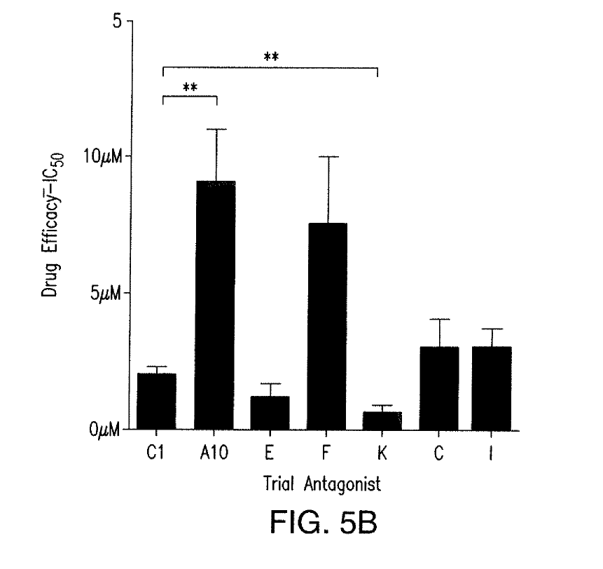

Numerous cellular assays were developed that were able to detect VIP responses absent of interference from non-specific vasopressin receptors using this CHO based system. VIPR2 is a Gs coupled receptor. Activation of VIPR2 by VIP leads to both an elevation in cAMP signaling and the recruitment of β-arrestin. A variety of combinations can be transiently transfect into these CHO cells for the assessment of cellular responses to VIP. For the BRET readout CHO cell lines expressing an YFP-Epac-RLuc8 (CAMYEL) BRET sensor were used to detect changes in cAMP. This construct includes Epac1, a guanine nucleotide exchange factor activated by direct binding of cAMP, fused to an enhanced YFP and RLuc8 allowing a change in BRET upon cAMP-induced conformational changes [7]. The key for medium to high throughput screening assays is the use of stable cell lines allowing the continuous assaying of multiple compounds with the ability to generate large numbers of cells for rapid use. In these lines VIPR2 was expressed as one of the stable cell lines assays. This was introduced into the CHO-Flp-IN to allow the generation of isogenic stable cell lines expressing high levels of VIPR2 under control of the CMV promoter. This has been introduced into this line together with the CAMYEL construct previously described. This allows for detection of VIPR2-specific responses to cAMP. EYFP fluorescence and RLuc8 luminescence is quantified in the presence of 5 μM light-emitting luciferin, coelenterazine H, using a PHERAstar (BMG) plate reader. BRET is calculated by quantifying the ratio of the light emitted by the acceptor, YFP (λ=525 nm), over the emission from the donor, RLuc8 (λ=485 nm). Representative traces from the VIPR2 cAMP assay with a battery of novel VIPR2 antagonists are shown in FIG. 5A-B.

In a second stable cellular assay, recruitment of mVenus-β-arrestin2 to VIPR2-RLuc8 is measured. Here mVenus was cloned onto the C-terminus of β-arrestin, while Rluc8 was cloned onto the C-terminus of VIPR2 allowing the donor and acceptor to be brought into proximity on recruitment of β-arrestin to activated VIPR2. This requires triplicate expression with G protein-coupled receptor (GPCR) kinase (GRK)-2. This assay is outlined in FIG. 6.

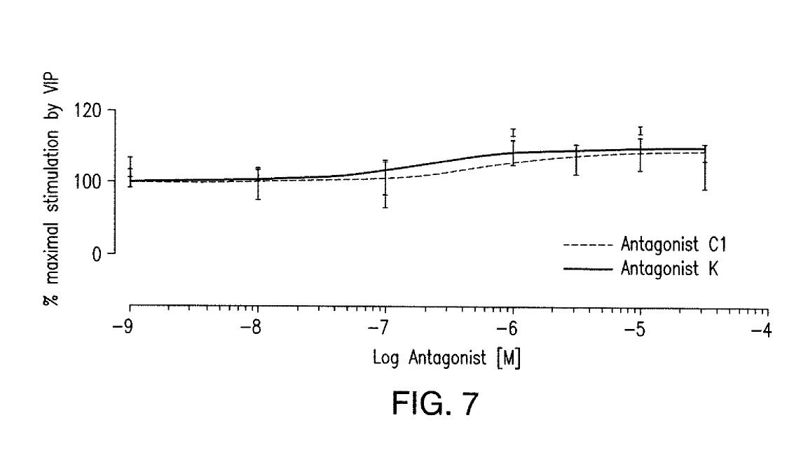

These assays are now being used for reliable and rapid quantification of VIPR2 responses to various antagonists under development for this receptor. This assay can also be used to evaluate agonists to VIPR2 and allosteric compounds, in addition to addresing the functional selectivity of different compounds, as it is possible to address multiple pathways effected by modulation of VIPR2 using this second messenger detection systems (cAMP and β-arrestin). This cellular assay for the detection of vasopressin based responses is applicable to multiple members of the Vasopressin family. To this end lines were also developed expressing CAMYEL together with VIPR1 and PACAP1R (both the human and mouse forms) using transient transfection. This allows for the confirmation of specificity of novel antagonists to the VIPR2 receptor in addition to providing the ability to assay VIPR1 and PACAPR1 antagonists, agonists and allosteric modulators. Results for VIPR2 antagonists C1 and K using the transient cell lines expressing hVIPR1 demonstrate the ability of this assay to determine VIPR₂ specificity (FIG. 7). These assays allow assays of VIPR2 antagonist, agonists and other compounds acting at vasopressin family receptors in a specific, selective, rapid and medium to high throughput manner.

### 6.4 Example 4

| for 500 ml | Volume |
|---|---|
| DMEM + 4500 mg L-Glucose, + L-glutamine, − Pyruvate | 500 ml |
| 10% FBS | 50 ml |
| Penicillin/Streptomycin | 5 ml |

1. Passage HEK cells at a daily doubling rate (1.25 in 10 dilution for a 3 day passage).
2. The day prior to transfection seed cells at 4 million per 10 cm dish.
Generating Stable Flp-In™ Expression Cell Lines, Selection of Stable Flp-1n™ Expression Cell Lines. The gene of interest will be expressed from pcDNA™5/FRT under the control of the human CMV promoter. Once generated the Flp-In™ expression with recombinant protein should be expressed constitutively. Reminder: Following cotransfection, Flp-In™ expression clones should become sensitive to Zeocin™; therefore, selection medium should not contain Zeocin™.
1. Cotransfect mammalian Flp-In™ host cells with a 9:1 ratio of pOG44:pcDNA™5/FRT plasmid DNA using the desired protocol. Include a plate with no pOG44 as a Flp recombination control, a plate of untransfected cells as a negative control, and the pcDNA™5/FRT/CAT plasmid as a positive control. Lipofectamine protocol-Per well of a 6 well plate. 1.2 μg DNA+200 μl Optimern in one vial (9:1 ratio of pOG44:pcDNA™5/FRT plasmid DNA). 2 μl Lipofectamine+200 μl Optimem (leave 5 mins to mix). Combine for 20 minutes.
2. 24 hours after transfection, wash the cells and add fresh medium to the cells.
3. 48 hours after transfection, split the cells into fresh medium, such that they are no more than 25% confluent. If the cells are too dense, the antibiotic will not kill the cells. Antibiotics work best on actively dividing cells.
4. Plate the trypsinized cells in the presence of hygromycin immediately (at the predetermined concentration for your cell line). This will ensure that ONLY the true transfectants survive.
5. Feed the cells with selective medium every 3-4 days until foci can be identified.
6. Pick 5-20 hygromycin-resistant foci and expand the cells. Verify that the pcDNA™5/FRT construct has integrated into the FRT site by testing each clone for Zeocin™ sensitivity and lack of β-galactosidase activity.
7. Select those clones that are hygromycin-resistant, Zeocin™-sensitive, and lacZ-, then assay for expression of your gene of interest.

| | Antibiotic Concentrations | | | |
|---|---|---|---|---|
| | Working Conc | Stock Solution | For 10 ml | For 50 ml |
| Blasticidin S | 1.5 μg/ml | 10 mg/ml | 1.5 μl | 7.5 μl |
| Hygromycin B | 100 μg/ml | 50 mg/ml | 20 μl | 100 μl |
| Zeocin | 500 μg/ml | 100 mg/ml | 50 μl | 250 μl |
| G418 | 250 μg/ml | 250 mg/ml | 10 μl | 50 μl |

PEI based transfection

### Reagents:

PEI (1 μg/μl)—PEI is Polyethylenimine 25 kD linear.
To make a stock solution:
1. Dissolve PEI in endotoxin-free dH₂O that has been heated to ˜80° C.
2. Let cool to room temperature.
3. Neutralize to pH 7.0, filter sterilize (0.22 μm), aliquot and store at −20° C.; a working stock can be kept at 4° C.

### Cell Transfection:

Prior to transfection bring all reagents to room temperature.
1. In a sterile tube dilute total plasmid DNA (˜20 μg) in 500 μl of serum-free DMEM.
2. In a separate sterile tube dilute total plasmid PEI (20 μg) in 500 μl of DMEM. (Vortex well prior and post addition of PEI).
3. Mix the solutions and leave for >15 minutes. Add evenly to 10 cm dish of CHO cells and leave for 24 hrs.
4. 24 hrs later replace media with fresh, pre-warmed supplemented media.

| Transfection Ratios for BRET | cAMP |
|---|---|
| Camyel vector | 6 μg |
| Receptor (e.g. VPAC2) | 1.5 μg |
| PC5 | to total 20 μg |

| Transfection Ratios for BRET | β-Arrestin |
|---|---|
| Venus-arr3 | 8 μg |
| SF-hVPAC2-Rluc8 | 0.05 μg |
| GRK5 | 5 μg |
| PC5 | to total 20 μg |

### BRET Assay

### Reagents:

Preparation of coelenterazine h.
1. Allow the vial of lyophilized Coelenterazine-H to equilibrate to ambient temperature before opening to avoid condensation. Protect from light.
2. Reconstitute coelenterazine h with methanol or ethanol. Do not dissolve in dimethylsulfoxide (DMSO).
3. Agitate gently until resuspended, potentially a few minutes.
4. Aliquot (volumes depend on expected rate of use) and store desiccated and protected from light at 20° C.
Prepare drugs as appropriate to assay in 96 well plate to facilitate direct transfer to assay wells.
Dilute in 1×PBS+NaHSO₃ (2 mg/50 ml PBS).
Each well in the final assay will contain:

| BRET assay wells Component | Volume |
|---|---|
| Cells | 45 μl |
| 5 μM coelenterazine H | 10 μl |
| Agonist/Antagonist | 45 μ1 |

### Leave at 4° C.

All stages can be carried out at Room Temperature under non-sterile conditions.
1. Carefully wash transfected CHO cells in 1×PBS and collect in 1 ml PBS.
2. Spin down pellet for 5 mins at 1 ref.
3. Resuspend in 600 μl/plate of PBS supplemented with 1×PBS with glucose (5 mM Glucose or 0.5 ml of 0.5 M in 50 ml of 1×PBS).
4. Quantify cell density using the BCA assay

| | 1 | 2 | 3 | 4 | 5 | 6 | 7 | 8 |
|---|---|---|---|---|---|---|---|---|
| | 0 μg | 5 μg | 10 μg | 15 μg | 20 μg | 25 μg | 30 μg | 35 μg |
| BSA (μl) | 0 | 2.5 | 5 | 7.5 | 10 | 12.5 | 15 | 17.5 |
| dH₂O (μl) | 25 | 22.2 | 10 | 17.5 | 15 | 12.5 | 10 | 7.5 |
| Samples | 25 | | | | | | | |

BSA at 2
mg/ml

Add 200 μl BCA per well and leave to incubate for 15-30 minutes at 37° C.
Quantify using the Polarstar OPTIMA plate reader.
Dilute cells as appropriate to allow for the correct number of curves with 20-40 μg of cells.
Running the BRET assay:
Guidelines plate setup:
1. Prepare two boxes complete with pipette tips for multichannel pipetting
2. Add 45 μl of cells to each well using the multichannel pipetter
3. At t=0 mins—Add 22.5 μl of antagonist row by row using the multichannel pipetter (dispense at timed intervals to match rate of plate reading 6 sees, 18 sees, 30 sees, 42 secs, 54 sees, 1 min 6 sees)
4. Prepare coelenterazine H, 13 μl in 1.3 ml of DPBS/NaBis per plate in a scintillation vial protected by foil.
5. At t=7 mins—Add 10 μl of coelenterazine H to each well at 1 second intervals
6. At t=15 mins—Add 22.5 μl of agonist row by row using the multichannel pipetter (dispense at timed intervals to match rate of plate reading 6 sees, 18 sees, 30 sees, 42 sees, 54 sees, 1 min 6 sees)
7. At t=16 mins 40 sees—Insert plate reader into the plate reader to initiate readout. (2 min readout)
8. At t=24 mins 40 sees—Re-insert plate reader into the plate reader to initiate readout. (10 min readout)

### Analysis:

1. Export the file into Microsoft excel
2. Calculate the BRET ratio

$$B R E T RATIO = \frac{λ525}{λ485}$$

3. The ratio should decrease in response to increased cAMP response
4. Transpose values to GraphPad ready layout and import data into GraphPad Prism
5. Analyze results

### 7. REFERENCES

[1]V. Vacic, S. McCarthy, D. Malhotra, F. Murray, H.-H. Chou, A. Peoples, et al., Duplications of the neuropeptide receptor gene VIPR2 confer significant risk for schizophrenia, Nature. 471 (2011) 499-503.
[2]B. Collado, M. J. Carmena, M. Sanchez-Chapado, A. Ruiz-Villaespesa, A. M. Bajo, A. B. Fernandez-Martinez, et al., Expression of vasoactive intestinal peptide and functional VIP receptors in human prostate cancer: antagonistic action of a growth-hormone-releasing hormone analog, Int. J. Oncol. 26 (2005) 1629-1635.
[3]R. P. Gomariz, Y. Juarranz, C. Abad, A. Arranz, J. Leceta, C. Martinez, VIP-PACAP system in immunity: new insights for multitarget therapy, Ann N Y Acad Sci. 1070 (2006) 51-74.
[4]M. Tsutsumi, T. H. Claus, Y. Liang, Y. Li, L. Yang, J. Zhu, et al., A potent and highly selective VPAC2 agonist enhances glucose-induced insulin release and glucose disposal: a potential therapy for type 2 diabetes, Diabetes. 51 (2002) 1453-1460.
[5]A. Chu, J. S. Caldwell, Y. A. Chen, Identification and Characterization of a Small Molecule Antagonist of Human VPAC2 Receptor, Molecular Pharmacology. 77 (2010) 95-101.
[6]B. K. Atwood, J. Lopez, J. Wager-Miller, K. Mackie, A. Straiker, Expression of G protein-coupled receptors and related proteins in HEK293, AtT20, BV2, and N18 cell lines as revealed by microarray analysis, BMC Genomics. 12 (2011) 14.
[7]L. I. Jiang, J. Collins, R. Davis, K. M. Lin, D. DeCamp, T. Roach, et al., Use of a cAMP BRET Sensor to Characterize a Novel Regulation of cAMP by the Sphingosine 1-Phosphate/G 13 Pathway, J Biol Chem. 282 (2007) 10576-10584.

Various patents and other publications are cited herein, the contents of which are hereby incorporated by reference in their entireties.

We claim:

1. A method for identifying an antagonist of VIPR2 comprising:
(a) contacting a VIPR2 agonist to a cell expressing a recombinant VIPR2 protein, wherein the cell does not express endogenous VIPR2 protein, and detecting the level of cAMP in the cell;
(b) contacting a candidate compound to the cell and detecting the level of cAMP in the cell;
(c) comparing the level of cAMP in (a) and (b); and
(d) selecting the candidate compound as a VIPR2 antagonist when the level of cAMP in (b) is less than the level of cAMP in (a).

2. The method of claim 1, wherein the cell expresses a Bioluminescence Resonance Energy Transfer (BRET) sensor, wherein binding of cAMP to the BRET sensor causes a detectable change in Bioluminescence Resonance Energy Transfer (BRET).

3. The method of claim 2, wherein the BRET sensor comprises a YFP-Epac-RLuc8(CAMYEL) BRET sensor.

4. The method of claim 1, wherein the cell is a CHO cell.

5. The method of claim 4, wherein the CHO cell is a CHO-Flp-IN CHO cell.

6. A method for identifying an agonist of VIPR2 comprising:
(a) contacting a candidate compound to a first cell expressing a recombinant VIPR2 protein, wherein the first cell does not express endogenous VIPR2 protein;
(b) detecting the level of cAMP in the first cell;
(c) comparing the level of cAMP in the first cell to the level of cAMP in a second cell expressing a recombinant VIPR2 protein not contacted with the candidate compound, wherein the second cell does not express endogenous VIPR2 protein;
(d) selecting the candidate compound as a VIPR2 agonist when the cAMP level in the first cell is greater than the level of cAMP in the second cell.

7. A method for identifying an antagonist of VIPR2 comprising:
(a) contacting a VIPR2 agonist to a cell expressing a recombinant VIPR2 protein, wherein the cell does not express endogenous VIPR2 protein, and detecting the level of β-arrestin recruited to the recombinant VIPR2 protein in the cell;
(b) contacting a candidate compound to the cell and detecting the level of β-arrestin recruited to the recombinant VIPR2 protein in the cell;
(c) comparing the level of β-arrestin recruited to the recombinant VIPR2 protein in (a) and (b); and
(d) selecting the candidate compound as a VIPR2 antagonist when the level of β-arrestin recruited to the recombinant VIPR2 protein in (b) is less than the level in (a).

8. The method of claim 7, wherein the cell expresses a Bioluminescence Resonance Energy Transfer (BRET) sensor, wherein recruitment of β-arrestin to the recombinant VIPR2 protein causes a detectable change in Bioluminescence Resonance Energy Transfer (BRET).

9. The method of claim 8, wherein the BRET sensor comprises an mVenus-β-arrestin2 construct and a VIPR2-RLuc8 construct.

10. The method of claim 7, wherein the cell is a CHO cell.

11. The method of claim 10, wherein the CHO cell is a CHO-Flp-IN CHO cell.

12. A method for identifying an agonist of VIPR2 comprising:
(a) contacting a candidate compound to a first cell expressing a recombinant VIPR2 protein, wherein the first cell does not express endogenous VIPR2 protein;
(b) detecting the level of β-arrestin recruited to the recombinant VIPR2 protein in the first cell;
(c) comparing the level of β-arrestin recruited to the recombinant VIPR2 protein in the first cell to the level of β-arrestin recruited to a recombinant VIPR2 protein in a second cell expressing a recombinant VIPR2 protein not contacted with the candidate compound, wherein the second cell does not express endogenous VIPR2 protein;
(d) selecting the candidate compound as a VIPR2 agonist when the level of β-arrestin recruited to the recombinant VIPR2 protein in the first cell is greater than the level in the second cell.
